SEQUENCE LISTING (41 sequences)

SEQ ID NO: 1 - DNA, 219 bp, Homo sapiens
  source 1..219: mol_type = other DNA
atgatcgaggttgtttgcaacgaccgtctggggaagaaggtccgcgttaaatgcaacacggatgataccatcggggaccttaagaagctgattgcagcccaaactggtacccgttggaacaagattgtcctgaagaagtggtacacgatttttaaggaccacgtgtctctgggggactatgaaatccacgatgggatgaacctggagctttattatcaa

SEQ ID NO: 2 - DNA, 1698 bp, Homo sapiens
  source 1..1698: mol_type = other DNA
atgttcgtgtccgatttccgcaaagagttctacgaggtggtccagagccagagggtccttctcttcgtggcctcggacgtggatgctctgtgtgcgtgcaagatccttcaggccttgttccagtgtgaccacgtgcaatatacgctggttccagtttctgggtggcaagaacttgaaactgcatttcttgagcataaagaacagtttcattattttattctcataaactgtggagctaatgtagacctattggatattcttcaacctgatgaagacactatattctttgtgtgtgacacccataggccagtcaatgtcgtcaatgtatacaacgatacccagatcaaattactcattaaacaagatgatgaccttgaagttcccgcctatgaagacatcttcagggatgaagaggaggatgaagagcattcaggaaatgacagtgatgggtcagagccttctgagaagcgcacacggttagaagaggagatagtggagcaaaccatgcggaggaggcagcggcgagagtgggaggcccggagaagagacatcctctttgactacgagcagtatgaatatcatgggacatcgtcagccatggtgatgtttgagctggcttggatgctgtccaaggacttgaatgacatgctgtggtgggccatcgttggactaacagaccagtgggtgcaagacaagatcactcaaatgaaatacgtgactgatgttggtgtcctgcagcgccacgtttcccgccacaaccaccggaacgaggatgaggagaacacactctccgtggactgcacacggatctcctttgagtatgacctccgcctggtgctctaccagcactggtccctccatgacagcctgtgcaacaccagctataccgcagccaggttcaagctgtggtctgtgcatggacagaagcggctccaggagttccttgcagacatgggtcttcccctgaagcaggtgaagcagaagttccaggccatggacatctccttgaaggagaatttgcgggaaatgattgaacagtctgcaaataaatttgggatgaaggacatgcgcgtgcagactttcagcattcattttgggttcaagcacaagtttctggccagcgacgtggtctttgccaccatgtctttgatggagagccccgagaaggatggctcagggacagatcacttcatccaggctctggacagcctctccaggagtaacctggacaagctgtaccatggcctggaactcgccaagaagcagctgcgagccacccagcagaccattgccagctgcctttgcaccaacctcgtcatctcccaggggcctttcctgtactgctctctcatggagggcactccagatgtcatgctgttctctaggccggcatccctaagcctgctcagcaaacacctgctcaagtcctttgtgtgttcgacaaagaaccggcgctgcaaactgctgcccctggtgatggctgcccccctgagcatggagcatggcacagtgaccgtggtgggcatccccccagagaccgacagctcggacaggaagaacttttttgggagggcgtttgagaaggcagcggaaagcaccagctcccggatgctgcacaaccattttgacctctcagtaattgagctgaaagctgaggatcggagcaagtttctggacgcacttatttccctcctgtcc

SEQ ID NO: 3 - DNA, 7803 bp, Homo sapiens
  source 1..7803: mol_type = other DNA
atgaagagaaaagtcgtgaatactcacaagctgagattgagtcctaatgaggaagccttcattttgaaggaagattatgaaagaaggcgaaaactaagattgctacaggttcgagaacaagaaagagatatcgccttacagataagagaagacataaaacagaggagaaatcaacaatttacacgtttggcagaggagctaagggcagaatgggaagaatcacaaactcagaaaatacagaacttggaaaaactgtatttggcaagtttaagaagtatgggagagggacatcgacaggccaaagaaaatgaacctgatttggatgctttggcacagcgggcagcagaaaggaaaagaaaagcagatttgaggcataaagaagccttgaaagtacagaaaaatcaaaaagaaatattactgaaacaaaaaacctggcatataaaagctcgaaaggaagcactgcttgtggaaaaagagagatcagccaaaattacaagtctgccacctcctcctccaactctttttgagaacatcgaagtaaaaagaatttctgcagtcaaaaccaatagttctacctaccatcatcttcacacttttgtgaatagagagacagacacaaaacggccagatgctcgtttggctgctgaagaggaagctaaacgattggaagaactacaaaaacaggcagcacaagagagaatggaacggtttgaaaaggcacatgtacggggattccaagcaatgaagaagatccatttggctcaaaatcaggagaaactaatgaaagaactcaaacagctacagcaagaggacctggcacgtaggagacagactgtagcacaaatgccaccacaactagttgaacttccatacaaacgcagtgaaatgaaagaagactggcagagagaattggaatttgcctttgaagatatgtacaatgcagacaggaaggtgaaagggaatctgattctgcaccttgaaccagagcccttgcccactgtgactaatcagatccaagatgaagagctggacctttcaatggaacaagaaaatttgggtgcagctgaagaccttccagtgacagaagctgaaatatgttctagtgaaacagatgttcccttggtaatgaagacccaacagattccttcaaaagttctttttaaaaaattattaaataagatccgaagccaaaaatctctctggacaattaaatctatgtctgaggatgaaagtgaaatgattacgactgttagtgaaattgagagtaaagcaccaacggttgagtcaggaacaattgccagcaaagagagaacgttatcctctgggcaggaacaagttgttgaaagtgatacactaacaattgagtctggaccacttgctagtgaagataaaccactttcgtgtggtacaaactctggaaaagaacaagaaataaatgagactctgcctatcacaactgtagctcagagttcagttctacttcatcctcaagaagcagcagccaggattagaatgtcagcaaggcagaaacagataatggaaatagaagagcagaagcaaaagcaattggaattacttgaacaaattgaacagcagaaattaagattagaaactgactgcttcagggctcagctggaagaagaaaaaagaaaaaaaactcaaccgactggggttggcattgctccagcatcatgccctgtaatttctgatgaagatagtcataggcagatgattcgtaactatcaacatcagcttttacaacaaaacaggttacacaggcagtctgttgaaacagccaggaaacaattacttgaatatcaaactatgttaaaaggaaggtgcccatcggtgtcagctccatcattgataactgattctgttatatcagtgccatcatggaaatctgagagaccgactgctatatcagagcattgggatcaaggtcagagactcaagttgagtcctaacaaataccaacccatacaacctatacagacctccaaattagaacaagatcattttcaggtagcgagacaaaatcactttccacaaagacaggtggaaacaacagaaacattacgcgcttcagatattttaaccaatcaagctttagaatcacaagaacatctaaggcaattctctcagactgaaacacaacagagagactataaattggtccccaaagattctgagacactttcaagggctttgtcacatgacaggcagctaatatcacaggatgctagaaaaatatctgaaacatttggggcaacaacttttcaaagtttagaatcccaacaattgttctcagagaatagtgaaaatatatcttaccatttaactgaaccttcttcatttgtaccactggtacctcagcattcttttagttctctgcctgttaaagttgagtcaggaaaaattcaagaacccttttcagccatgagcaaaagtacagtttccacaagccattctataatcagccaaatgcatgataggcctttgctgccgtcagagaatatcacagcccagcaaggtaatatgaaggccctccaagaacagttagacctacagaagaaagttcttcaggcaactcaggaagctcaggaacagttgcttttgtgcaaacagaaagaagtggaacagcaaacgggcctctcggtattccttcccttggtaactccagattcatctgctttattgccttctgccaaagcagatttggggagaatccaggaatcttcaccaaccaagaataatattgcagtttcctcagaccatcatgtgatctcacaacttcaggataagcgtttgagtctttcacagcctatcctatcacagcaaaataattttaaatttctccaagagcagttgaatattcagaaggatagccttcaggctaggcgagaagcccaggaagtattgtatgtacataaacagagtgaattggatagaagagtatgttccgaacaggctgagccctctttcccatttcaggtagctcagcatacatttacttcactaccatctgctgatacaaaatctggaaaaatacaggagcaacattcatctaagagcgagaaaggacttgtttcatgccaatctgacatccccatatctcaggatgggtctttgagtttcctacagcagttcctacctctacatgatagtttgaagttgctccaagaacagttgactaaacagagggatactcttcaggctaggcatgaagctcaggtggaattacttttacatagacaaagagatttgggggacagtaagtctgggctggtgagctcttcatcctcaccagtggttgttcagcattcagttgcttcacaagcttctgctaaagctgagcctaggagaattcaggagctttatttatctgagaaggagaatgtaggtccctcctgtcatttgataatcccaacatttcaggataagtctcttagttttccacagcatagcctggcacagcaagaaaatttgacaatactccaagaacagtcacaaatacaaagggtaatacttggtgctaaagaaggaactcaggaatttgtacacacagaaagtgaattggagaaaagaatttcttctgaacagactggcacctcctcatccctttcccaggtggatgaatctgagagattccaggaatgtatatcaatcaagagtgacagtaccattcccttaagccatcctaagatcccaagatgtcaggaaagacttttgagagtttcacaacatatgctacctctacaagataatttggaggaacaccaagcatggctagacactgagaaagaagcctttcatttcagccagaaaacccaagaaaatacatcttctgaacaaactggttcatcttcattcataccccagttggtacagctttcatttacttcgttagcttcagctgagtctggcacaatcctggaacctctttttacagagagtgaaagtaaaattttttcaagccaccttcagatcccacaattgcaggataggcttttgaggatatcgcaacttatccagcctcaacaagataatttgaaggcacttcaagaacagttagctacacagagagaagccatcattctagctagacaagaagctcgggaagaattacttttacatcagagtgaatgggagggaagaatatctcccgagcaggttgacacctcttccttacccctagtaccacagcattcattcgcctcattacctcttaatgaatctgaaagaaaccaagaaccatgttcaattaacagtgataatatagtatcctcaggtcactcagagataccaacattgcctgatgggctgttgggtttatcacatcttgttttacctcaacaagataatttgattgcacttgaagaacacttgcatgcacagacagatttccttccttctattgagaaaacccagaaagaattggttttgtcaaaaccatgtaaatttgaggaaaaggtatcttctgagcattttatccagtctcaccatggtgatttgcaggcacttcaacagcagttagatacacagaagaaagccattcgatctatacaggaagtccaagaagaattgcttttgcaaagattaagtgaattggagaaaagggtatcatctgaacaagtttgctcctcttcatttgtatcccaggtgcctgttgctgactctgaaagaacccagaagtctttcccaaccaaaagtaatgatactcttccctcaagtcatcgtgagattccaagattacaggatagacttttgagtttatcaaagcctattctgcctcagcaagataatatgacagcacaattggatgcacaaagggaagtgatgtattcttatgagaaaccccaggaagaactgtctttaaacaaacaaagaaagttgaacaaaagtgaatctgctgagcatactatcccctctttgtttctacccaaggaaacagagcattcgtttattccactaccttttgcagaagctaaacctaaaagcacttgtgaattgtattcatcccagaatgaacatgcagcccccccaagtaatcctgtgatcccagggtttcaagatagacttttgagtttttcacagtctgtcttaactcagcaagataacttgggacttcagaaacagttggatctacaaagagaagttctgcattatagccagaaagcccaggaaaaattgcttgtacagagacaaacagcattgcagcagcagatacagaaacatgaagagactttgaaggatttctttaaagacagtcagataagtaagcccacagttgaaaatgatttaaaaacccagaagatggggcagctcagagactggtttcctaatacacaagacctagcaggaaatgatcaagaaaatattaggcatgcagataggaacaactctgatgataatcatttggcttcagaagatactagtgccaagcaaagtggtgagcatctggagaaagatctggggagaagatcctcaaagccacctgtagcaaaagtcaaatgtggtttggacttaaaccagcatgaacttagtgctatacaagaagtagagtcaccagcaattggcagaacttctatactaggtaaaccaggtatttatgaagacagagaccccctgcgagtctcaataagccgagaacaaagtttctttgggagcccactggcccatgatccgtttagttgtcttcaactggttggccaagagaatgtctgtggtgatgactatgatgaagcagttaagctgaaggaatctgttgttgaaaatcatgcagtgttaagttatgctgtggaggaagaacatgcatatttgggtccaactgtgaagccagatgataaggctaaaacactgtcttatgaaccattatcttcagcaactgtttccactgggagccttttaagttatgaaaacacagatttgagccttacagatccagagtcattttcagagcacatggatgatagcaagcaagaatctaccaccagtaaagaagaggaaacaaatattataagttccatagttccttcaacacaagatatttatcagcggcagaactcttcagacgttcataaatctctgttgcctgcagtggatgaaactacatgtggtcacacacactttcagcaaatgatagacaagtacattaatgaagcaaatttgatacctgaaaaaacagatttgcaagaattggaacacatttttcctaatttgcatcatcagctgtttaaacccttagaaccacatccagattttgacttatcatcatcatcctctgggatttctccagacaacagagacttttaccagagatcagattcttcatctgaaagccactgtgctactggattatccaaaagtacagtttatttcacagcactgaggaggaccagcatgcattcttctcttaacacaagtccgaatcaacaacctgacactaacttggctcatgttggagctcacagttttgctacagaaaatattattgggggatctgaacaatgttttgaacagcttcagccagaatattcttcacaggaggagagccagcatgctgatctaccaagtatttttagcattgaagcaagagattcttcccaaggcatgaaaaatcagaactatccctctgaagaacatactgaaatattacaaaacaagaaaaaaattgttcatttccagctttctataggaaacttaagttcagtctacagttcatctgatgaagctaatgtatttgatcagttaaatgtacagcatagcactccatgtggttctaactctagtgagtgctcaacaaaacaccaactagaaagcagaaaggaaagtatgggctttgaagaactatcaaaaagaggggttgttacaatgttacaaagtcaaggactcattgaagataataaaaatgaaacctgtagggttttagacataaatccacaggtagaggaaactgactctcgattatgtgtaagaacagtggagatgggaacttcaattcaggcaccatattccttaactactcaaaatgaaaaatattttgagaattcagctgaaacagacattccaaaaatcaccaaaaaactatctcaactaggagaatcagagctttttgcaagttctggatcattttcattacagagctctataccagtctgggaaacagaaactggccatggtataatggaagaaccagaacttactttaataagcaccactgataccagtattgctgaaatggattttgcaaatttaaccctagaagagaagagcgagaatgaagcaaaatgcttctttcaggtgagtgagtttctgcctcttgtatcagcaacagaagcctcagattatccagctgtatcagaactttccatagaaaaaccaaggacagcatctacagaaacccctcgcaggcttacacctgtaccagggagcttacaagaagcatttataaagaggaaaaaatcatttatggagagatcccaccagaggcagaaagaaataaggaataaaattcatgtctctgaaaattctcaaatcaaaacagttaaagagaaaccatctataagttcatctgtgagtcgtctaaagggcgtgaataaagtcagagcatcttttcctgaagacagaaagactacacaggctctaaggcaccaaaggggtctaaggttatacaatcaactagctgaagtgaaacaacaaaaggaagaaaaaacaaaacaagaagcttatgcccaaaacagagcaagggcaaaagaattccataagaaaacactagagaaacttcgagccaaaaatacatgc

SEQ ID NO: 4 - DNA, 666 bp, Homo sapiens
  source 1..666: mol_type = other DNA
atgttgagccgggcagtgtgcggcaccagcaggcagctggctccggttttggggtatctgggctccaggcagaagcacagcctccccgacctgccctacgactacggcgccctggaacctcacatcaacgcgcagatcatgcagctgcaccacagcaagcaccacgcggcctacgtgaacaacctgaacgtcaccgaggagaagtaccaggaggcgttggccaagggagatgttacagcccagatagctcttcagcctgcactgaagttcaatggtggtggtcatatcaatcatagcattttctggacaaacctcagccctaacggtggtggagaacccaaaggggagttgctggaagccatcaaacgtgactttggttcctttgacaagtttaaggagaagctgacggctgcatctgttggtgtccaaggctcaggttggggttggcttggtttcaataaggaacggggacacttacaaattgctgcttgtccaaatcaggatccactgcaaggaacaacaggccttattccactgctggggattgatgtgtgggagcacgcttactaccttcagtataaaaatgtcaggcctgattatctaaaagctatttggaatgtaatcaactgggagaatgtaactgaaagatacatggcttgcaaaaag

SEQ ID NO: 5 - DNA, 381 bp, Homo sapiens
  source 1..381: mol_type = other DNA
atgtacatcgacagctacaacagccgcggcttcatgataaacggaaaccgcgtgctcggcccctgcgctctgctcccgcactcggtggtgcagtggaacgtgggatcccaccaggacatcaccgaagacagcttttccctcttctggttgctggagccccggatagagatcgtggtggtggggactggagaccggaccgagaggctgcagtcccaggtgcttcaagccatgaggcagcggggcattgctgtggaagtgcaggacacgcccaatgcctgtgccaccttcaacttcctgtgtcatgaaggccgagtaactggagctgctctcatccctccaccaggagggacttcacttacatctttgggccaagctgctcaa

SEQ ID NO: 6 - DNA, 630 bp, Homo sapiens
  source 1..630: mol_type = other DNA
atgtggactctcgggcgccgcgcagtagccggcctcctggcgtcacccagcccagcccaggcccagaccctcacccgggtcccgcggccggcagagttggccccactctgcggccgccgtggcctgcgcaccgacatcgatgcgacctgcacgccccgccgcgcaagttcgaaccaacgtggcctcaaccagatttggaatgtcaaaaagcagagtgtctatttgatgaatttgaggaaatctggaactttgggccacccaggctctctagatgagaccacctatgaaagactagcagaggaaacgctggactctttagcagagttttttgaagaccttgcagacaagccatacacgtttgaggactatgatgtctcctttgggagtggtgtcttaactgtcaaactgggtggagatctaggaacctatgtgatcaacaagcagacgccaaacaagcaaatctggctatcttctccatccagtggacctaagcgttatgactggactgggaaaaactgggtgtactcccacgacggcgtgtccctccatgagctgctggccgcagagctcactaaagccttaaaaaccaaactggacttgtcttccttggcctattccggaaaagatgct

SEQ ID NO: 7 - DNA, 273 bp, Homo sapiens
  source 1..273: mol_type = other DNA
atgtggactctcgggcgccgcgcagtagccggcctcctggcgtcacccagcccagcccaggcccagaccctcacccgggtcccgcggccggcagagttggccccactctgcggccgccgtggcctgcgcaccgacatcgatgcgacctgcacgccccgccgcgcaagttcgaaccaacgtggcctcaaccagatttggaatgtcaaaaagcagagtgtctatttgatgaatttgaggaaatctggaactttgggccacccaggagtggtgtct

SEQ ID NO: 8 - DNA, 1359 bp, Homo sapiens
  source 1..1359: mol_type = other DNA
atgaagctactaaccagagccggctctttctcgagattttattccctcaaagttgcccccaaagttaaagccacagctgcgcctgcaggagcaccgccacaacctcaggaccttgagtttaccaagttaccaaatggcttggtgattgcttctttggaaaactattctcctgtatcaagaattggtttgttcattaaagcaggcagtagatatgaggacttcagcaatttaggaaccacccatttgctgcgtcttacatccagtctgacgacaaaaggagcttcatctttcaagataacccgtggaattgaagcagttggtggcaaattaagtgtgaccgcaacaagggaaaacatggcttatactgtggaatgcctgcggggtgatgttgatattctaatggagttcctgctcaatgtcaccacagcaccagaatttcgtcgttgggaagtagctgaccttcagcctcagctaaagattgacaaagctgtggcctttcagaatccgcagactcatgtcattgaaaatttgcatgcagcagcttaccggaatgccttggctaatcccttgtattgtcctgactataggattggaaaagtgacatcagaggagttacattacttcgttcagaaccatttcacaagtgcaagaatggctttgattggacttggtgtgagtcatcctgttctaaagcaagttgctgaacagtttctcaacatgaggggtgggcttggtttatctggtgcaaaggccaactaccgtggaggtgaaatccgagaacagaatggagacagtcttgtccatgctgcttttgtagcagaaagtgctgtcgcgggaagtgcagaggcaaatgcatttagtgttcttcagcatgtcctcggtgctgggccacatgtcaagaggggcagcaacaccaccagccatctgcaccaggctgttgccaaggcaactcagcagccatttgatgtttctgcatttaatgccagttactcagattctggactctttgggatttatactatctcccaggccacagctgctggagatgttatcaaggctgcctataatcaagtaaaaacaatagctcaaggaaacctttccaacacagatgtccaagctgccaagaacaagctgaaagctggatacctaatgtcagtggagtcttctgagtgtttcctggaagaagtcgggtcccaggctctagttgctggttcttacatgccaccatccacagtccttcagcagattgattcagtggctaatgctgatatcataaatgcggcaaagaagtttgtttctggccagaagtcaatggcagcaagtggaaatttgggacatacaccttttgttgatgagttg

SEQ ID NO: 9 - protein, 73 aa, Homo sapiens
MIEVVCNDRLGKKVRVKCNTDDTIGDLKKLIAAQTGTRWNKIVLKKWYTIFKDHVSLGDYEIHDGMNLELYYQ

SEQ ID NO: 10 - protein, 566 aa, Homo sapiens
MFVSDFRKEFYEVVQSQRVLLFVASDVDALCACKILQALFQCDHVQYTLVPVSGWQELETAFLEHKEQFHYFILINCGANVDLLDILQPDEDTIFFVCDTHRPVNVVNVYNDTQIKLLIKQDDDLEVPAYEDIFRDEEEDEEHSGNDSDGSEPSEKRTRLEEEIVEQTMRRRQRREWEARRRDILFDYEQYEYHGTSSAMVMFELAWMLSKDLNDMLWWAIVGLTDQWVQDKITQMKYVTDVGVLQRHVSRHNHRNEDEENTLSVDCTRISFEYDLRLVLYQHWSLHDSLCNTSYTAARFKLWSVHGQKRLQEFLADMGLPLKQVKQKFQAMDISLKENLREMIEQSANKFGMKDMRVQTFSIHFGFKHKFLASDVVFATMSLMESPEKDGSGTDHFIQALDSLSRSNLDKLYHGLELAKKQLRATQQTIASCLCTNLVISQGPFLYCSLMEGTPDVMLFSRPASLSLLSKHLLKSFVCSTKNRRCKLLPLVMAAPLSMEHGTVTVVGIPPETDSSDRKNFFGRAFEKAAESTSSRMLHNHFDLSVIELKAEDRSKFLDALISLLS

SEQ ID NO: 11 - protein, 566 aa, Homo sapiens
MFVSDFRKEFYEVVQSQRVLLFVASDVDALCACKILQALFQCDHVQYTLVPVSGWQELETAFLEHKEQFHYFILINCGANVDLLDILQPDEDTIFFVCDTHRPVNVVNVYNDTQIKLLIKQDDDLEVPAYEDIFRDEEEDEEHSGNDSDGSEPSEKRTRLEEEIVEQTMRRRQRREWEARRRDILFDYEQYEYHGTSSAMVMFELAWMLSKDLNDMLWWAIVGLTDQWVQDKITQMKYVTDVGVLQRHVSRHNHRNEDEENTLSVDCTRISFEYDLRLVLYQHWSLHDSLCNTSYTAARFKLWSVHGQKRLQEFLADMGLPLKQVKQKFQAMDISLKENLREMIEESANKFGMKDMRVQTFSIHFGFKHKFLASDVVFATMSLMESPEKDGSGTDHFIQALDSLSRSNLDKLYHGLELAKKQLRATQQTIASCLCTNLVISQGPFLYCSLMEGTPDVMLFSRPASLSLLSKHLLKSFVCSTKNRRCKLLPLVMAAPLSMEHGTVTVVGIPPETDSSDRKNFFGRAFEKAAESTSSRMLHNHFDLSVIELKAEDRSKFLDALISLLS

SEQ ID NO: 12 - protein, 2601 aa, Homo sapiens
MKRKVVNTHKLRLSPNEEAFILKEDYERRRKLRLLQVREQERDIALQIREDIKQRRNQQFTRLAEELRAEWEESQTQKIQNLEKLYLASLRSMGEGHRQAKENEPDLDALAQRAAERKRKADLRHKEALKVQKNQKEILLKQKTWHIKARKEALLVEKERSAKITSLPPPPPTLFENIEVKRISAVKTNSSTYHHLHTFVNRETDTKRPDARLAAEEEAKRLEELQKQAAQERMERFEKAHVRGFQAMKKIHLAQNQEKLMKELKQLQQEDLARRRQTVAQMPPQLVELPYKRSEMKEDWQRELEFAFEDMYNADRKVKGNLILHLEPEPLPTVTNQIQDEELDLSMEQENLGAAEDLPVTEAEICSSETDVPLVMKTQQIPSKVLFKKLLNKIRSQKSLWTIKSMSEDESEMITTVSEIESKAPTVESGTIASKERTLSSGQEQVVESDTLTIESGPLASEDKPLSCGTNSGKEQEINETLPITTVAQSSVLLHPQEAAARIRMSARQKQIMEIEEQKQKQLELLEQIEQQKLRLETDCFRAQLEEEKRKKTQPTGVGIAPASCPVISDEDSHRQMIRNYQHQLLQQNRLHRQSVETARKQLLEYQTMLKGRCPSVSAPSLITDSVISVPSWKSERPTAISEHWDQGQRLKLSPNKYQPIQPIQTSKLEQDHFQVARQNHFPQRQVETTETLRASDILTNQALESQEHLRQFSQTETQQRDYKLVPKDSETLSRALSHDRQLISQDARKISETFGATTFQSLESQQLFSENSENISYHLTEPSSFVPLVPQHSFSSLPVKVESGKIQEPFSAMSKSTVSTSHSIISQMHDRPLLPSENITAQQGNMKALQEQLDLQKKVLQATQEAQEQLLLCKQKEVEQQTGLSVFLPLVTPDSSALLPSAKADLGRIQESSPTKNNIAVSSDHHVISQLQDKRLSLSQPILSQQNNFKFLQEQLNIQKDSLQARREAQEVLYVHKQSELDRRVCSEQAEPSFPFQVAQHTFTSLPSADTKSGKIQEQHSSKSEKGLVSCQSDIPISQDGSLSFLQQFLPLHDSLKLLQEQLTKQRDTLQARHEAQVELLLHRQRDLGDSKSGLVSSSSSPVVVQHSVASQASAKAEPRRIQELYLSEKENVGPSCHLIIPTFQDKSLSFPQHSLAQQENLTILQEQSQIQRVILGAKEGTQEFVHTESELEKRISSEQTGTSSSLSQVDESERFQECISIKSDSTIPLSHPKIPRCQERLLRVSQHMLPLQDNLEEHQAWLDTEKEAFHFSQKTQENTSSEQTGSSSFIPQLVQLSFTSLASAESGTILEPLFTESESKIFSSHLQIPQLQDRLLRISQLIQPQQDNLKALQEQLATQREAIILARQEAREELLLHQSEWEGRISPEQVDTSSLPLVPQHSFASLPLNESERNQEPCSINSDNIVSSGHSEIPTLPDGLLGLSHLVLPQQDNLIALEEHLHAQTDFLPSIEKTQKELVLSKPCKFEEKVSSEHFIQSHHGDLQALQQQLDTQKKAIRSIQEVQEELLLQRLSELEKRVSSEQVCSSSFVSQVPVADSERTQKSFPTKSNDTLPSSHREIPRLQDRLLSLSKPILPQQDNMTAQLDAQREVMYSYEKPQEELSLNKQRKLNKSESAEHTIPSLFLPKETEHSFIPLPFAEAKPKSTCELYSSQNEHAAPPSNPVIPGFQDRLLSFSQSVLTQQDNLGLQKQLDLQREVLHYSQKAQEKLLVQRQTALQQQIQKHEETLKDFFKDSQISKPTVENDLKTQKMGQLRDWFPNTQDLAGNDQENIRHADRNNSDDNHLASEDTSAKQSGEHLEKDLGRRSSKPPVAKVKCGLDLNQHELSAIQEVESPAIGRTSILGKPGIYEDRDPLRVSISREQSFFGSPLAHDPFSCLQLVGQENVCGDDYDEAVKLKESVVENHAVLSYAVEEEHAYLGPTVKPDDKAKTLSYEPLSSATVSTGSLLSYENTDLSLTDPESFSEHMDDSKQESTTSKEEETNIISSIVPSTQDIYQRQNSSDVHKSLLPAVDETTCGHTHFQQMIDKYINEANLIPEKTDLQELEHIFPNLHHQLFKPLEPHPDFDLSSSSSGISPDNRDFYQRSDSSSESHCATGLSKSTVYFTALRRTSMHSSLNTSPNQQPDTNLAHVGAHSFATENIIGGSEQCFEQLQPEYSSQEESQHADLPSIFSIEARDSSQGMKNQNYPSEEHTEILQNKKKIVHFQLSIGNLSSVYSSSDEANVFDQLNVQHSTPCGSNSSECSTKHQLESRKESMGFEELSKRGVVTMLQSQGLIEDNKNETCRVLDINPQVEETDSRLCVRTVEMGTSIQAPYSLTTQNEKYFENSAETDIPKITKKLSQLGESELFASSGSFSLQSSIPVWETETGHGIMEEPELTLISTTDTSIAEMDFANLTLEEKSENEAKCFFQVSEFLPLVSATEASDYPAVSELSIEKPRTASTETPRRLTPVPGSLQEAFIKRKKSFMERSHQRQKEIRNKIHVSENSQIKTVKEKPSISSSVSRLKGVNKVRASFPEDRKTTQALRHQRGLRLYNQLAEVKQQKEEKTKQEAYAQNRARAKEFHKKTLEKLRAKNTC

SEQ ID NO: 13 - protein, 222 aa, Homo sapiens
MLSRAVCGTSRQLAPVLGYLGSRQKHSLPDLPYDYGALEPHINAQIMQLHHSKHHAAYVNNLNVTEEKYQEALAKGDVTAQIALQPALKFNGGGHINHSIFWTNLSPNGGGEPKGELLEAIKRDFGSFDKFKEKLTAASVGVQGSGWGWLGFNKERGHLQIAACPNQDPLQGTTGLIPLLGIDVWEHAYYLQYKNVRPDYLKAIWNVINWENVTERYMACKK

SEQ ID NO: 14 - protein, 127 aa, Homo sapiens
MYIDSYNSRGFMINGNRVLGPCALLPHSVVQWNVGSHQDITEDSFSLFWLLEPRIEIVVVGTGDRTERLQSQVLQAMRQRGIAVEVQDTPNACATFNFLCHEGRVTGAALIPPPGGTSLTSLGQAAQ

SEQ ID NO: 15 - protein, 210 aa, Homo sapiens
MWTLGRRAVAGLLASPSPAQAQTLTRVPRPAELAPLCGRRGLRTDIDATCTPRRASSNQRGLNQIWNVKKQSVYLMNLRKSGTLGHPGSLDETTYERLAEETLDSLAEFFEDLADKPYTFEDYDVSFGSGVLTVKLGGDLGTYVINKQTPNKQIWLSSPSSGPKRYDWTGKNWVYSHDGVSLHELLAAELTKALKTKLDLSSLAYSGKDA

SEQ ID NO: 16 - protein, 91 aa, Homo sapiens
MWTLGRRAVAGLLASPSPAQAQTLTRVPRPAELAPLCGRRGLRTDIDATCTPRRASSNQRGLNQIWNVKKQSVYLMNLRKSGTLGHPGVVS

SEQ ID NO: 17 - protein, 453 aa, Homo sapiens
MKLLTRAGSFSRFYSLKVAPKVKATAAPAGAPPQPQDLEFTKLPNGLVIASLENYSPVSRIGLFIKAGSRYEDFSNLGTTHLLRLTSSLTTKGASSFKITRGIEAVGGKLSVTATRENMAYTVECLRGDVDILMEFLLNVTTAPEFRRWEVADLQPQLKIDKAVAFQNPQTHVIENLHAAAYRNALANPLYCPDYRIGKVTSEELHYFVQNHFTSARMALIGLGVSHPVLKQVAEQFLNMRGGLGLSGAKANYRGGEIREQNGDSLVHAAFVAESAVAGSAEANAFSVLQHVLGAGPHVKRGSNTTSHLHQAVAKATQQPFDVSAFNASYSDSGLFGIYTISQATAAGDVIKAAYNQVKTIAQGNLSNTDVQAAKNKLKAGYLMSVESSECFLEEVGSQALVAGSYMPPSTVLQQIDSVANADIINAAKKFVSGQKSMAASGNLGHTPFVDEL

SEQ ID NO: 18 - DNA, 219 bp, synthetic construct
  source 1..219: mol_type = other DNA
atgatcgaggtggtgtgcaacgataggctgggcaagaaggtgagggtgaagtgcaataccgatgacaccatcggcgacctgaagaagctgatcgccgcccagacaggaacacgctggaataaaatcgtgctgaagaagtggtacaccatcttcaaggatcacgtgtctctgggcgactatgagatccacgacggcatgaatctggagctgtactatcag

SEQ ID NO: 19 - protein, 7 aa, synthetic construct
YNGTIFF

SEQ ID NO: 20 - protein, 7 aa, synthetic construct
RSIFFLK

SEQ ID NO: 21 - protein, 7 aa, synthetic construct
LVSYFGI

SEQ ID NO: 22 - protein, 7 aa, synthetic construct
NPIIFFL

SEQ ID NO: 23 - protein, 7 aa, synthetic construct
YNGIIVF

SEQ ID NO: 24 - protein, 7 aa, synthetic construct
LVPYNHI

SEQ ID NO: 25 - protein, 7 aa, synthetic construct
LVSYNGM

SEQ ID NO: 26 - protein, 7 aa, synthetic construct
VSYHGII

SEQ ID NO: 27 - protein, 7 aa, synthetic construct
YNGIMFF

SEQ ID NO: 28 - protein, 7 aa, synthetic construct
YNGIILF

SEQ ID NO: 29 - protein, 7 aa, synthetic construct
GIQFFTK

SEQ ID NO: 30 - protein, 736 aa, Adeno-associated dependoparvovirus A
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 31 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQYNGTIFFAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 32 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQRSIFFLKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 33 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQLVSYFGIAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 34 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQNPIIFFLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 35 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQYNGIIVFAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 36 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQLVPYNHIAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 37 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQLVSYNGMAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 38 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQVSYHGIIAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 39 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQYNGIMFFAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 40 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQYNGIILFAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 41 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQGIQFFTKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

(19) United States
(12) Patent Application Publication
(10) Pub. No.: US 2025/0367329 A1
(43) Pub. Date: Dec. 4, 2025

(54) GENE THERAPY FOR TREATING MITOCHONDRIAL STRESS

(71) Applicant: YISSUM RESEARCH DEVELOPMENT COMPANY OF THE HEBREW UNIVERSITY OF JERUSALEM LTD., Jerusalem (IL)

(72) Inventors: Yaakov NAHMIAS, Shoresh (IL); Avner EHRLICH, Mazkeret Batia (IL); Konstantinos IOANNIDIS, Piraeus (GR)

(73) Assignee: YISSUM RESEARCH DEVELOPMENT COMPANY OF THE HEBREW UNIVERSITY OF JERUSALEM LTD., Jerusalem (IL)

(21) Appl. No.: 19/293,448

(22) Filed: Aug. 7, 2025

Related U.S. Application Data
(63) Continuation of application No. PCT/IL2024/050151, filed on Feb. 8, 2024.
(60) Provisional application No. 63/444,054, filed on Feb. 8, 2023.

(51) Int. Cl. A61K 48/00; C07K 14/005; C07K 14/435; C12N 15/86

(52) U.S. Cl. CPC A61K 48/0058; C07K 14/005; C07K 14/435; C12N 15/86; C07K 2319/01; C12N 2750/14122; C12N 2750/14143

(57) ABSTRACT

Nanoparticles comprising a nucleic acid molecule encoding human ubiquitin-like protein 5 (UBL5) or human UBL5 protein are provided. Methods of treating a disease, disorder or condition characterized by mitochondrial stress are provided. Expression vectors, nucleic acid molecules, peptides, pharmaceutical compositions and methods of identifying a gene for use in gene therapy, targeting an agent to a CD44 expressing cell and producing a therapeutic nanoparticle are also provided.

### CROSS REFERENCE TO RELATED APPLICATIONS

This application is a Bypass Continuation of PCT Patent Application No. PCT/IL2024/050151 having International filing date of Feb. 8, 2024, which claims the benefit of priority of U.S. Provisional Patent Application No. 63/444,054, filed on Feb. 8, 2023, the contents of which are all incorporated herein by reference in their entirety.

### REFERENCE TO AN ELECTRONIC SEQUENCE LISTING

The contents of the electronic sequence listing (HUJI-P-097-PCT.xml; Size: 63,092 bytes; and Date of Creation: Feb. 6, 2024) is herein incorporated by reference in its entirety.

### FIELD OF INVENTION

The present invention is in the field of gene therapy and disease associated with mitochondrial stress.

### BACKGROUND OF THE INVENTION

Mitochondrial stress is a dysfunction of central carbon metabolism leading to energy depletion, the production of free radicals in affected tissues, and subsequent inflammatory response. Mitochondrial stress can be produced due to nutrient overload, infection, DNA damage, or biochemical insult. This mechanism is at the basis of multiple disease states including fatty liver disease, obesity, diabetes, atherosclerosis, chronic kidney disease, bone marrow failure, dementia, Alzheimer's as well as rare genetic diseases such as Leptin Receptor Deficiency and Fanconi anemia. Mitochondrial stress is often accompanied by lipid accumulation leading to lipotoxicity and inflammatory response. There are currently no therapeutics targeting this core disease mechanism and such therapeutics are greatly needed.

### SUMMARY OF THE INVENTION

The present invention provides nanoparticles comprising a nucleic acid molecule encoding human ubiquitin-like protein 5 (UBL5) or human UBL5 protein. Nanoparticles comprising a nucleic acid molecule encoding a human protein selected from Cell division control protein 45 homolog (CDC45), Centrosomal protein of 295 kDa (CEP295), Superoxide dismutase [Mn], mitochondrial (SOD2), NADH dehydrogenase [ubiquinone] 1 alpha subcomplex assembly factor 3 (NDUFAF3), Frataxin, mitochondrial (FXN), and Cytochrome b-c1 complex subunit 2, mitochondrial (UQCRC2) or a human protein selected from CDC45, CEP295, SOD2, NDUFAF3, FXN, and UQCRC2 are also provided. Methods of treating a disease, disorder or condition characterized by mitochondrial stress are provided. Nucleic acid molecules comprising an optimized sequence encoding UBL5 is provided. Peptides that bind to CD44 are provided. Capsid fusion proteins are provided. Expression vectors and pharmaceutical compositions comprising the nanoparticles, nucleic acid molecules, peptides and fusion proteins of the invention are also provided. Methods of targeting an agent to a CD44 expressing cell are provided. Methods of producing a therapeutic agent are provided. Methods of identifying a gene for use in gene therapy are also provided.

According to a first aspect, there is provided a nanoparticle comprising a shell and an aqueous core, wherein the aqueous core comprises at least one of:
a. a nucleic acid molecule, wherein the nucleic acid molecule comprises a promoter operably linked to an open reading frame encoding human Ubiquitin-like protein 5 (UBL5); and
b. a human UBL5 polypeptide.

According to some embodiments, the human UBL5 comprises the amino acid sequence of SEQ ID NO: 9 or a variant thereof comprising at least 85% identity to SEQ ID NO: 9 and which retains unfolded protein response (UPR) functionality in mitochondria.

According to some embodiments, the open reading frame comprises the nucleotide sequence of SEQ ID NO: 1 or a variant thereof with at least 80% identity to SEQ ID NO: 1.

According to some embodiments, the open reading frame comprises the nucleotide sequence of SEQ ID NO: 18 or a variant thereof comprising at least 85% identity to SEQ ID NO: 18 and at least 80% identity to SEQ ID NO: 1.

According to some embodiments, the variant of SEQ ID NO: 18 encodes SEQ ID NO: 9.

According to some embodiments, the open reading frame consists of SEQ ID NO: 18.

According to some embodiments, the promoter is a heterologous promoter.

According to some embodiments, the nanoparticle is selected from a viral nanoparticle, a lipid nanoparticle and a synthetic nanoparticle.

According to some embodiments, the nanoparticle is an adeno associated viral (AAV) nanoparticle.

According to some embodiments, the AAV nanoparticle is an AAV9 nanoparticle.

According to some embodiments, the nanoparticle comprises a CD44 targeting peptide on the shell, wherein the CD44 targeting peptide is selected from YNGTIFF (SEQ ID NO: 19), RSIFFLK (SEQ ID NO: 20), LVSYFGI (SEQ ID NO: 21), NPIIFFL (SEQ ID NO: 22), YNGIIVF (SEQ ID NO: 23), LVPYNHI (SEQ ID NO: 24), LVSYNGM (SEQ ID NO: 25), VSYHGII (SEQ ID NO: 26), YNGIMFF (SEQ ID NO: 27), YNGIILF (SEQ ID NO: 28) and GIQFFTK (SEQ ID NO: 29).

According to some embodiments, the nanoparticle is a viral nanoparticle and the CD44 targeting peptide is inserted into a capsid of the viral nanoparticle.

According to some embodiments, the CD44 targeting peptide is inserted between glutamine 588 and alanine 589, and wherein positions are with respect to SEQ ID NO: 30.

According to some embodiments, the nanoparticle comprises a capsid fusion protein comprising the CD44 targeting peptide comprising an amino acid sequence selected from: SEQ ID NO: 31-41.

According to another aspect, there is provided a nucleic acid molecule comprising the nucleic acid sequence provided in SEQ ID NO: 18.

According to some embodiments, the nucleic acid molecule comprises a promoter operatively linked to the nucleic acid sequence.

According to some embodiments, the nucleic acid molecule is an expression vector.

According to another aspect, there is provided a pharmaceutical composition comprising a nanoparticle of the invention or a nucleic acid molecule of the invention and a pharmaceutically acceptable carrier, excipient or adjuvant.

According to some embodiments, the pharmaceutical composition is formulated for systemic administration to the subject.

According to some embodiments, the systemic administration is selected from intravenous, intramuscular, subcutaneous, and intraperitoneal administration.

According to another aspect, there is provided a method of treating a disease, disorder or condition characterized by mitochondrial stress in a subject in need thereof, the method comprising administering to the subject a pharmaceutical composition of the invention, thereby treating a disorder or condition characterized by mitochondrial stress.

According to another aspect, there is provided a method of treating a disease, disorder or condition characterized by mitochondrial stress in a subject in need thereof, the method comprising increasing expression of a protein selected from Ubiquitin-like protein 5 (UBL5), Cell division control protein 45 homolog (CDC45), Centrosomal protein of 295 kDa (CEP295), Superoxide dismutase [Mn], mitochondrial (SOD2), NADH dehydrogenase [ubiquinone]1 alpha subcomplex assembly factor 3 (NDUFAF3), Frataxin, mitochondrial (FXN), and Cytochrome b-cl complex subunit 2, mitochondrial (UQCRC2) in a diseased cell of the subject, thereby treating a disease characterized by mitochondrial stress, optionally wherein the subject is a human.

According to some embodiments, the mitochondrial stress comprises abnormal lipid accumulation in disease cells or diseased tissue of the subject, optionally wherein the abnormal lipid accumulation comprises the presence of lipid droplets in the diseased cells or diseased tissue at a level that is increased as compared to healthy cells or tissue.

According to some embodiments, the mitochondrial stress comprises abnormal basal metabolic rate in disease cells or diseased tissue of the subject, optionally wherein the abnormal basal metabolic rate comprises the reduction of oxygen consumption in the disease cells or diseased tissue.

According to some embodiments, the disease, disorder or condition is selected from a neuromuscular disease, an immune disease, a hematological disease, a cardiovascular disease, a neurodegenerative disease, a metabolic disorder or disease, a renal disorder, a dermatological condition, a cognitive disorder or a skeletomuscular condition.

According to some embodiments, the disease, disorder or condition is selected from atherosclerosis, hyperlipidemia, hypercholesterolemia, hypertriglyceridemia, Alzheimer's disease, Parkinson's disease, Huntington's disease, amyotrophic lateral sclerosis (ALS), diabetes mellitus, obesity, fatty liver disease, metabolic syndrome, kidney disease, rhytides, elastosis, lentigines, dementia, sarcopenia, and bone marrow failure diseases.

According to some embodiments, the disease, disorder or condition is selected from a hematological disease and a metabolic disease.

According to some embodiments, the disease, disorder or condition is a bone marrow failure disease or anemia.

According to some embodiments, the bone marrow failure disease is selected from the group consisting of: Fanconi Anemia, Aplastic Anemia, Diamond-Blackfan Anemia, Dyskeratosis Congenita/Telomere Biology Disorders, GATA2 Deficiency, Myelodysplastic Syndrome, Paroxysmal Nocturnal Hemoglobinuria, Pearson's Disease, SAMD9/SAMD9L Germline Mutations, Severe Congenital Neutropenia, and Shwachman-Diamond Syndrome.

According to some embodiments, the bone marrow failure disease is Fanconi Anemia.

According to some embodiments, the disease, disorder or condition is a metabolic disease.

According to some embodiments, the metabolic disease is selected from metabolic syndrome, fatty liver disease, obesity, insulin resistance and diabetes mellitus.

According to some embodiments, the metabolic disease is non-alcoholic fatty liver disease (NAFLD).

According to some embodiments, the metabolic disease is insulin resistance, diabetes or both.

According to some embodiments, the metabolic disease is obesity.

According to some embodiments, the obesity is a rare genetic disease of obesity.

According to some embodiments, the rare genetic disease of obesity is selected from the group consisting of: Bardet-Biedl syndrome (BBS), Alstrom syndrome, Proopiomelanocortin (POMC) deficiency, Leptin receptor (LEPR) deficiency, Leptin (LEP) deficiency, Proprotein convertase subtilisin/kexin type 1 (PCSK1) deficiency, Steroid receptor coactivator-1 (SRC1) deficiency, and SH2B adaptor protein 1 (SH2B1) deficiency.

According to some embodiments, the rare obesity disease is Leptin receptor (LEPR) deficiency or Leptin (LEP) deficiency.

According to some embodiments, the increasing comprises administering to the subject a nucleic acid vector encoding the protein.

According to some embodiments, the vector comprises a cDNA sequence encoding the protein, wherein the cDNA sequence is devoid of introns.

According to some embodiments, the increasing comprises administering the protein to the subject.

According to some embodiments, the increasing comprises administering to the subject an agonist of the protein.

According to some embodiments, the increasing comprises administering to the subject a pharmaceutical composition comprising a nanoparticle comprising a shell and an aqueous core, wherein the aqueous core comprises at least one of: a nucleic acid molecule, wherein the nucleic acid molecule comprises a promoter operatively linked to an open reading frame encoding the protein and the protein.

According to some embodiments, the disease is a bone marrow failure disease and the nanoparticle comprises a CD44 targeting peptide on the shell, wherein the CD44 targeting peptide is selected from SEQ ID NO: 19-29.

According to some embodiments, the method comprises receiving disease cells from the subject, increasing expression of the protein in the extracted cells and returning the extracted cells to the subject.

According to some embodiments, the increasing expression in the extracted cells comprises delivering into a cytoplasm and/or nucleus of the extracted cells at least one of: a nucleic acid vector encoding the protein, the protein and an agonist of the protein.

According to some embodiments, the method is devoid of a step measuring expression of the protein or a nucleic acid encoding the protein in disease cells of the subject.

According to some embodiments, the subject does not possess diseased cells with decreased expression of the protein as compared to heathy cells of the same tissue or cell type.

According to some embodiments, the increasing comprises increasing expression of the protein in the diseased cell beyond the expression level in healthy cells of the same tissue or cell type as the diseased cell.

According to some embodiments, the treating comprises decreasing lipid accumulation in diseased cells or diseased tissue of the subject.

According to another aspect, there is provided a nanoparticle comprising a shell and an aqueous core, wherein the aqueous core comprises at least one of:
a. a nucleic acid molecule, wherein the nucleic acid molecule comprises a promoter operatively linked to an open reading frame encoding a human protein selected from CDC45, CEP295, SOD2, NDUFAF3, FXN, and UQCRC2; and
b. a human protein selected from CDC45, CEP295, SOD2, NDUFAF3, FXN, and UQCRC2.

According to some embodiments, the open reading frame encodes CDC45 and comprises SEQ ID NO: 2, encodes CEP295 and comprises SEQ ID NO: 3, encodes SOD2 and comprises SEQ ID NO: 4, encodes NDUFAF3 and comprises SEQ ID NO: 5, encodes FXN and comprises SEQ ID NO: 6 or 7 or encodes UQCRC2 and comprises SEQ ID NO: 8.

According to some embodiments, the nucleic acid molecule is selected from a lentiviral vector, an adenoviral vector, an adeno-associated viral vector, and a plasmid.

According to another aspect, there is provided a pharmaceutical composition comprising a nanoparticle of the invention and a pharmaceutically acceptable carrier, excipient or adjuvant.

According to some embodiments, the pharmaceutical composition is for use in the performance of a method of the invention.

According to another aspect, there is provided a peptide comprising an amino acid sequence selected from the group consisting of SEQ ID NO: 19-29.

According to some embodiments, the peptide comprises 7-30 amino acids.

According to some embodiments, the peptide consists of an amino acid sequence selected from the group consisting of: SEQ ID NO: 19-29.

According to some embodiments, the peptide is for use in targeting to CD44 expressing cells.

According to another aspect, there is provided a capsid fusion protein comprising an amino acid sequence of a viral capsid protein and an amino acid sequence selected from SEQ ID NO: 19-29.

According to some embodiments, the sequence selected from SEQ ID NO: 19-29 is present in a coat protein domain of the viral capsid protein.

According to some embodiments, the sequence selected from SEQ ID NO: 19-29 is inserted between glutamine 588 and alanine 589 of the sequence of a viral capsid protein, and wherein positions are with respect to SEQ ID NO: 30.

According to some embodiments, the capsid fusion protein comprises or consists of an amino acid sequence selected from: SEQ ID NO: 31-40.

According to another aspect, there is provided a nucleic acid molecule, encoding a capsid fusion protein of the invention.

According to another aspect, there is provided a nanoparticle comprising a peptide of the invention or a capsid fusion protein of the invention.

According to some embodiments, the nanoparticle is for use in treating a bone marrow failure disease, wherein the nanoparticle further comprises an agent suitable for treating the bone marrow failure disease.

According to another aspect, there is provided a method of targeting an agent to a CD44 expressing cell in a subject, the method comprising producing a pharmaceutical composition comprising the agent and a peptide of the invention or a capsid fusion protein of the invention and administering the pharmaceutical composition to the subject, thereby targeting an agent to a CD44 expressing cell.

According to another aspect, there is provided a method of producing a therapeutic nanoparticle that targets to CD44 expressing cells, the method comprising:
a. providing a nanoparticle comprising a peptide of the invention on a surface of the nanoparticle; and
b. loading the nanoparticle with a drug;
thereby producing a therapeutic nanoparticle that targets to CD44 expressing cells.

According to another aspect, there is provided a method of identifying a gene for use in gene therapy to treat a disease, disorder or condition characterized by mitochondrial stress, the method comprising:
a. receiving a population of cells;
b. decreasing expression of a plurality of genes in the population of cells wherein each cell has decreased expression of only one gene of the plurality to produce a population of knockdown cells;
c. placing the population of knockdown cells in a condition of metabolic stress;
d. selecting a cell of the population of knockdown cells with a negative phenotype; and
e. identifying the gene of the plurality of genes with decreased expression in the selected cell;
thereby identifying a gene for use in gene therapy.

According to some embodiments, the population of cell is of the same cell type as is affected by the disease, disorder or condition.

According to some embodiments, the decreasing comprises a molecular screen performed in the received population of cells.

According to some embodiments, the screen is a genome-wide or pathway-wide CRISPR knockout screen.

According to some embodiments, the cells are human cells.

According to some embodiments, the cell are energetic cells, optionally wherein energetic cells are defined by an oxygen consumption rate (OCR) of greater than 50 pmol/min/10≡cells, an OCR (pmol/min)/extracellular acidification rate (ECAR) (mpH/min) greater than 1 or both.

According to some embodiments, the energetic cells are E6/E7LOW Hepatocytes.

According to some embodiments, the negative phenotype is increased intracellular lipid accumulation in the population of knockdown cells or decreased oxygen consumption in the population of knockdown cells, wherein increased and decreased is as compared to non-knocked-down cells, optionally wherein the increased intracellular lipid accumulation comprises the presence of lipid droplets in the population of knockdown cells at a level that is increased as compared to non-knocked-down cells.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1C: (1A) Micrographs of E6/E7Low hepatocytes grown under control or high fat and high glucose medium (HFG) emulating western-style diet. Perilipin 2 (PLIN2/ADRP) immunostaining of control and HFG cells is shown in green (first and second columns). PLIN2 outlines lipid droplets showing macro and microvesicular steatosis. Nile Red staining of lipids is shown in yellow (third column). (1B) Micrograph of Nile Red staining of lipids in hepatocytes grown under high glucose medium (HG) emulating hyperglycemia is shown in yellow. Both experiments show robust responses in both male and female hepatocytes. (1C) Bar chart of bioenergetic analysis of hepatocytes grown under control and HFG medium. The basal metabolic rate of steatotic (fatty) hepatocytes is significantly lower than the control.

FIGS. 2A-2B: (2A-2B) Volcano plots of sgRNAs enriched and depleted in hepatocytes sorts as overly steatotic (fatty) and or overly lean after (2A) HFG and (2B) HG treatment. Knockdown of UQCRC2 for example prevents lipid accumulation and is thus enriched in lean hepatocytes. Beta-scores and p.value pairs are calculated for each gene.

FIGS. 3A-3B: (3A) A bar graph showing the pathways associated with sgRNAs found to be different in steatosis. Cell cycle, lipid metabolism, and DNA metabolites appear to be common hits. (3B) Bubble plot showing the high-ranking genes selected for further testing. Beta-score (color-coded) and p-value (size-coded) are presented per condition.

FIGS. 4A-4D: (4A) Bar chart of normalized lipid content per gene shows sgRNAs that changed lipid accumulation in hepatocytes under control conditions. UBL5, DHX36, CDC45 and UQCRC2 increase lipids upon knockdown. Data normalized to control. p.values are color coded. Samples in quadruplicates. Error bars present ±SD. (4B) Bar chart of normalized lipid content per gene shows sgRNAs that changed lipid accumulation in hepatocytes grown under western-style diet (HFG). Many more genes become significant (dark bar), with UBL5, CDC45 and UQCRC2 increasing significance. Data normalized to control. p.values are color coded. Samples in quadruplicates. Error bars present ±SD. (4C) Bar chart of cell numbers per gene shows sgRNAs that affected cell survival or proliferation in hepatocytes grown under western-style diet (HFG). Genes like RPL3 and MCM6 showed a worrying drop in cell survival. Data normalized to control. p.values are color coded. Samples in quadruplicates. Error bars present ±SD. (4D) Micrographs of Nile Red lipid staining of hepatocytes grown in HFG medium with knockout of the listed genes. Genes whose knockout produced the greatest increase and the greatest reduction in lipid accumulation are shown.

FIG. 5: Bar graphs of relative expression of the various genes in control liver biopsies and biopsies from subjects with early stage or moderate NAFLD. Analysis of GSE193084.

FIGS. 6A-6B: (6A) Micrographs of lipid content in hepatocytes with overexpression CDC45 or UBL5 during HFG culture. Reduced lipid accumulation is observed. (6B) Bar graph quantifying the lipid content in the control, CDC45 or UBL5 overexpression hepatocyte showing significant decrease in lipid content of the cells.

FIGS. 7A-7C: (7A) Line graph depicting mitochondrial stress analysis on SeaHorse Bioanalyzer. Data shows lower basal metabolic rate, and high mitochondrial stress, in cells from Fanconi Anemia patients compared to the same cell line in which the FANCD mutation has been genetically corrected. (7B) Dot plot of SeaHorse Bioanalyzer experiment showing the expression of UBL5 in Fanconi Anemia using the TD01-VVL5 virus restored basal metabolic rate and mitochondrial max capacity. (7C) Bar graphs showing cellular viability of Fanconi Anemia cells exposed to a low dose (left) and a high dose (right) of the DNA damaging agent mitomycin C (MMC). Cells treated TD01-VVL5 virus treatment in white bar. Fanconi Anemia cells showed significant increase in survival following UBL5 expression.

FIGS. 8A-8B: (8A) Bar graph of DNA damage as determined by comet assay in gene corrected cells, Fanconi Anemia cells, and Fanconi Anemia cells treated with TD01-VVL5. Data shows the UBL5 correction of mitochondrial stress allows Fanconi Anemia cells to repair DNA damage. (8B) Micrographs showing comets of gene corrected cells, Fanconi Anemia cells, and Fanconi Anemia cells treated with TD01-VVL5 during the recovery phase.

FIGS. 9A-9B: Dot plots of (9A) general cell abundances and (9B) immune cell abundances in the bone marrow of wild-type mouse, mouse under bone marrow stress (BMF) and bone marrow stress mice treated with TD01-VVL5.

FIGS. 10A-10C: (10A) Micrographs of human liver organoids exposed to high dietary lipids (HFG) for 5 weeks and treated with TD02-OVL5 or an empty control virus. TD02-OVL5 expression of UBL5 caused significant decrease in lipid accumulation (10B) Micrograph of Masson Trichrome staining of organoids receiving TD02-OVL5 or an empty control virus following 5-week exposure to high dietary lipids (HFG). TD02-OVL5 expression of UBL5 caused significant decrease in collagen deposition and fibrosis. (10C) TEM images of human liver organoids exposed to high dietary lipids (HFG) and treated with TD02-OVL5 or an empty control virus. White arrows indicate mitochondria. TD02-OVL5 treated cells show intact mitochondria with ordered cristae while empty treated organoids display swollen mitochondria with disordered cristae.

FIGS. 11A-11C: (11A) Bar graph of oxygen consumption rate of human liver organoids exposed to high fat diet (HFG) for 5 weeks and treated with TD02-OVL5 or an empty control virus. Data shows increased basal metabolic rate following UBL5 expression. (11B) Bar graph of intracellular lipid accumulation in human liver organoids exposed to high fat diet (HFG) for 5 weeks and treated with TD02-OVL5 or an empty control virus. Data shows decreased lipid accumulation following UBL5 expression. (11C) Bar graph of glucose concentration in human liver organoids exposed to high fat diet (HFG) for 5 weeks and treated with TD02-OVL5 or an empty control virus. Data shows increased insulin sensitivity following UBL5 expression.

FIG. 12: Dot graph of mouse body weight over the course of the experiment in control mice, Ob/Ob mice, and Ob/Ob mice that received TD02-OVL5. UBL5 expression allows Ob/Ob mice to retain lean body weight.

FIGS. 13A-13C: (13A) Dot graph of liver weight in control mice, Ob/Ob mice, and Ob/Ob mice that received TD02-OVL5. UBL5 expression significantly reduced liver weight. (13B) Photo of livers (upper) and micrographs of liver histology (lower) from Ob/Ob mice and Ob/Ob mice that received TD02-OVL5. Data showed a significant reduction in lipid accumulation and NAFLD following UBL5 expression. (13C) Dot graph of AST and ALT liver enzyme levels in control mice, Ob/Ob mice, and Ob/Ob mice that received TD02-OVL5. UBL5 reduction in liver damage marks lower hepatic steatosis.

FIGS. 14A-14B: (14A) Dot graph of kidney weight in control mice, Ob/Ob mice, and Ob/Ob mice that received TD02-OVL5. UBL5 expression significantly reduced kidney weight. (14B) Micrographs of kidney morphology in Ob/Ob mice and Ob/Ob mice that received TD02-OVL5. Micrograph shows hallmarks of diabetic nephropathies, such as mesangial expansion, and glomerular basement membrane thickening. UBL5 expression relieves these effects.

FIGS. 15A-15B: (15A) Dot plot of circulating metabolite levels in control mice, Ob/Ob mice, and Ob/Ob mice that received TD02-OVL5. UBL5 expression resolved hyperglycemia, hypercholesterolemia, and hypertriglyceridemia, (15B) Micrograph of a pancreatic islet in a TD02-OVL5 treated Ob/Ob mice. Staining of insulin is shown.

FIG. 16: Bar graph of AAV adjusted relative quantitation of UBL5 expression for the non-optimized coding sequence and the optimized coding sequence. N=6 for both conditions.

FIG. 17: qPCR analysis comparing UBL5 expression in PBMCs exposed to TD01-VVL5 with and without a CD44 binding peptide integrated into the capsid (p<0.01; n=3).

### DETAILED DESCRIPTION OF THE INVENTION

The present invention, in some embodiments, provides nanoparticles comprising a nucleic acid molecule encoding human ubiquitin-like protein 5 (UBL5) or human UBL5 protein. Methods of treating a disease, disorder, or condition characterized by mitochondrial stress are also provided, as are expression vectors, nucleic acid molecules, peptides, pharmaceutical compositions, and methods of identifying a gene for use in gene therapy, targeting an agent to a CD44 expressing cell and producing a therapeutic nanoparticle.

The invention is based, at least in part on multiple genome-wide CRISPR-Cas9 knockout screen the inventors carried out in over 600 million primary hepatocytes. Genome wide screens require massive amounts of cells and were limited to tumors or cell lines that show a glycolytic phenotype characterized by low mitochondrial activity. The inventors previously developed a genetic mechanism that allows the conditional expansion of primary hepatocytes for 25 generations, allowing the cells to retain a high level of mitochondrial activity and liver-specific function. This technology allowed the inventors to screen over 600 million metabolically active cells and identify essential and restricting genes essential for stress-induced lipid accumulation. The inventors then cross-referenced the data to human patients, as well as genetic screens performed in other cell types to define stress-specific and -enriched regulators. The inventors thus identified the following regulators of metabolic stress UBL5, CDC45, UQCRC2, INO80, SRD5A1, CEP295, SOD2, NDUFAF3 and FXN. Ectopic expression or inhibition of these regulators resolved the metabolic stress in primary human cells.

### Nanoparticles

By a first aspect, there is provided a nanoparticle comprising a nucleic acid molecule that encodes a protein selected from Ubiquitin-like protein 5 (UBL5), Cell division control protein 45 homolog (CDC45), Centrosomal protein of 295 kDa (CEP295), Superoxide dismutase [Mn], mitochondrial (SOD2), NADH dehydrogenase [ubiquinone]1 alpha subcomplex assembly factor 3 (NDUFAF3), Frataxin, mitochondrial (FXN), and Cytochrome b-cl complex subunit 2, mitochondrial (UQCRC2).

By another aspect, there is provided a nanoparticle comprising a protein selected from UBL5, CDC45, CEP295, SOD2, NDUFAF3, FXN, and UQCRC2.

In some embodiments, the protein is a human protein. In some embodiments, the nucleic acid molecule encodes UBL5. In some embodiments, the protein is UBL5. In some embodiments, the nucleic acid molecule encodes CDC45. In some embodiments, the protein is CDC45. In some embodiments, the nucleic acid molecule encodes CEP295. In some embodiments, the protein is CEP295. In some embodiments, the nucleic acid molecule encodes SOD2. In some embodiments, the protein is SOD2. In some embodiments, the nucleic acid molecule encodes NDUFAF3. In some embodiments, the protein is NDUFAF3.

In some embodiments, the nucleic acid molecule encodes FXN. In some embodiments, the protein is FXN. In some embodiments, the nucleic acid molecule encodes UQCRC2. In some embodiments, the protein is UQCRC2.

In some embodiments, the nucleic acid molecule comprises an open reading frame encoding the protein. In some embodiments, an open reading frame is a coding region. In some embodiments, the open reading frame comprises a cDNA sequence encoding the protein. In some embodiments, the open reading frame is devoid of introns. In some embodiments, the nucleic acid molecule is devoid of introns. In some embodiments, the nucleic acid molecule is an artificial nucleic acid molecule. In some embodiments, the nucleic acid molecule comprises the gene that encodes the protein. In some embodiments, the gene is devoid of introns.

In some embodiments, the nucleic acid molecule is an expression vector. In some embodiments, the expression vector is an expression vector of the invention. In some embodiments, the expression vector is a mammalian expression vector. In some embodiments, the expression vector is a viral expression vector. In some embodiments, the expression vector is an AAV vector. In some embodiments, the nucleic acid molecule comprises a transcription regulatory element. In some embodiments, the transcription regulatory element is a promoter. In some embodiments, the promoter is a constitutively active promoter. In some embodiments, the promoter is a human promoter. In some embodiments, the promoter is a viral promoter. In some embodiments, the promoter is a heterologous promoter. In some embodiments, the promoter is not the endogenous promoter of the gene encoding the protein. In some embodiments, the transcription regulatory element is operably linked to the open reading frame. In some embodiments, the transcription regulatory element is operably linked to a gene encoding the protein.

The term “operably linked” is intended to mean that the nucleotide sequence of interest is linked to the regulatory element or elements in a manner that allows for expression of the nucleotide sequence (e.g., in an in vitro transcription/translation system or in a host cell when the vector is introduced into the host cell). In some embodiments, the promoter is active in a target cell. In some embodiments, the target cell is a diseased cell. In some embodiments, the promoter allows for expression of the nucleotide sequence in a target cell.

The term “promoter” as used herein refers to a group of transcriptional control modules that are clustered around the initiation site for an RNA polymerase i.e., RNA polymerase II. Promoters are composed of discrete functional modules, each consisting of approximately 7-20 bp of DNA, and containing one or more recognition sites for transcriptional activator or repressor proteins. In some embodiments, the promoter is a constitutive promoter. In some embodiments, the promoter is an inducible promoter. In some embodiments, the promoter is a tissue specific promoter. In some embodiments, the promoter is a cell or cell type specific promoter. In some embodiments, the cell is the target cell.

In some embodiments, the open reading frame encodes Ubiquitin-like protein 5 (UBL5) protein. In some embodiments, UBL5 protein comprises or consists of the amino acid sequence MIEVVCNDRLGKKVRVKCNTDDTIGDLKKLIAAQTGTRWNKIVLKKWYTIFKDH VSLGDYEIHDGMNLELYYQ (SEQ ID NO: 9). In some embodiments, the UBL5 coding sequence (CDS) comprises or consists of the nucleic acid sequence atgatcgaggttgtttgcaacgaccgtctggggaagaaggtccgcgttaaatgcaacacggatgataccatcggggaccttaaga agctgattgcagcccaaactggtacccgttggaacaagattgtcctgaagaagtggtacacgatttttaaggaccacgtgtctctgg gggactatgaaatccacgatgggatgaacctggagctttattatcaa (SEQ ID NO: 1). Human UBL5 mRNA sequences can be found in RefSeq accession numbers: NM_024292 and NM_001048241. Human UBL5 protein sequences can be found in RefSeq accession numbers: NP_077268, NP_077268.1, NP_001041706 and NP_001041706.1. The UBL5 protein can also be found at Uniprot #: Q9BZL1. Any of the sequences described herein can be used for expression of UBL5 as part of a molecule of the invention or a method of the invention.

In some embodiments, the open reading frame encodes Cell division control protein 45 homolog (CDC45) protein. In some embodiments, CDC45 comprises or consists of the amino acid sequence MFVSDFRKEFYEVVQSQRVLLFVASDVDALCACKILQALFQCDHVQYTLVPVSG WQELETAFLEHKEQFHYFILINCGANVDLLDILQPDEDTIFFVCDTHRPVNVVNVY NDTQIKLLIKQDDDLEVPAYEDIFRDEEEDEEHSGNDSDGSEPSEKRTRLEEEIVEQ TMRRRQRREWEARRRDILFDYEQYEYHGTSSAMVMFELAWMLSKDLNDMLWW AIVGLTDQWVQDKITQMKYVTDVGVLQRHVSRHNHRNEDEENTLSVDCTRISFE YDLRLVLYQHWSLHDSLCNTSYTAARFKLWSVHGQKRLQEFLADMGLPLKQVK QKFQAMDISLKENLREMIEQSANKFGMKDMRVQTFSIHFGFKHKFLASDVVFATM SLMESPEKDGSGTDHFIQALDSLSRSNLDKLYHGLELAKKQLRATQQTIASCLCTN LVISQGPFLYCSLMEGTPDVMLFSRPASLSLLSKHLLKSFVCSTKNRRCKLLPLVM AAPLSMEHGTVTVVGIPPETDSSDRKNFFGRAFEKAAESTSSRMLHNHFDLSVIEL KAEDRSKFLDALISLLS (SEQ ID NO: 10). In some embodiments, CDC45 protein comprises or consists of the amino acid sequence MFVSDFRKEFYEVVQSQRVLLFVASDVDALCACKILQALFQCDHVQYTLVPVSG WQELETAFLEHKEQFHYFILINCGANVDLLDILQPDEDTIFFVCDTHRPVNVVNVY NDTQIKLLIKQDDDLEVPAYEDIFRDEEEDEEHSGNDSDGSEPSEKRTRLEEEIVEQ TMRRRQRREWEARRRDILFDYEQYEYHGTSSAMVMFELAWMLSKDLNDMLWW AIVGLTDQWVQDKITQMKYVTDVGVLQRHVSRHNHRNEDEENTLSVDCTRISFE YDLRLVLYQHWSLHDSLCNTSYTAARFKLWSVHGQKRLQEFLADMGLPLKQVK QKFQAMDISLKENLREMIEESANKFGMKDMRVQTFSIHFGFKHKFLASDVVFATM SLMESPEKDGSGTDHFIQALDSLSRSNLDKLYHGLELAKKQLRATQQTIASCLCTN LVISQGPFLYCSLMEGTPDVMLFSRPASLSLLSKHLLKSFVCSTKNRRCKLLPLVM AAPLSMEHGTVTVVGIPPETDSSDRKNFFGRAFEKAAESTSSRMLHNHFDLSVIEL KAEDRSKFLDALISLLS (SEQ ID NO: 11). In some embodiments, the CDC45 CDS comprises or consists of the nucleic acid sequence atgttcgtgtccgatttccgcaaagagttctacgaggtggtccagagccagagggtccttctcttcgtggcctcggacgtggatgctc tgtgtgcgtgcaagatccttcaggccttgttccagtgtgaccacgtgcaatatacgctggttccagtttctgggtggcaagaacttgaa actgcatttcttgagcataaagaacagtttcattattttattctcataaactgtggagctaatgtagacctattggatattcttcaacctgat gaagacactatattctttgtgtgtgacacccataggccagtcaatgtcgtcaatgtatacaacgatacccagatcaaattactcattaaa caagatgatgaccttgaagttcccgcctatgaagacatcttcagggatgaagaggaggatgaagagcattcaggaaatgacagtg atgggtcagagccttctgagaagcgcacacggttagaagaggagatagtggagcaaaccatgcggaggaggcagcggcgaga gtgggaggcccggagaagagacatcctctttgactacgagcagtatgaatatcatgggacatcgtcagccatggtgatgtttgagc tggcttggatgctgtccaaggacttgaatgacatgctgtggtgggccatcgttggactaacagaccagtgggtgcaagacaagatc actcaaatgaaatacgtgactgatgttggtgtcctgcagcgccacgtttcccgccacaaccaccggaacgaggatgaggagaaca cactctccgtggactgcacacggatctcctttgagtatgacctccgcctggtgctctaccagcactggtccctccatgacagcctgtg caacaccagctataccgcagccaggttcaagctgtggtctgtgcatggacagaagcggctccaggagttccttgcagacatgggt cttcccctgaagcaggtgaagcagaagttccaggccatggacatctccttgaaggagaatttgcgggaaatgattgaacagtctgc aaataaatttgggatgaaggacatgcgcgtgcagactttcagcattcattttgggttcaagcacaagtttctggccagcgacgtggtc tttgccaccatgtctttgatggagagccccgagaaggatggctcagggacagatcacttcatccaggctctggacagcctctccag gagtaacctggacaagctgtaccatggcctggaactcgccaagaagcagctgcgagccacccagcagaccattgccagctgcct ttgcaccaacctcgtcatctcccaggggcctttcctgtactgctctctcatggagggcactccagatgtcatgctgttctctaggccgg catccctaagcctgctcagcaaacacctgctcaagtcctttgtgtgttcgacaaagaaccggcgctgcaaactgctgcccctggtga tggctgcccccctgagcatggagcatggcacagtgaccgtggtgggcatccccccagagaccgacagctcggacaggaagaa cttttttgggagggcgtttgagaaggcagcggaaagcaccagctcccggatgctgcacaaccattttgacctctcagtaattgagct gaaagctgaggatcggagcaagtttctggacgcacttatttccctcctgtcc (SEQ ID NO: 2). Human CDC45 mRNA sequences can be found in RefSeq accession numbers: NM_003504, NM001369291, NM_001178010 and NM_001178011. Human CDC45 protein sequences can be found in RefSeq accession numbers: NP_003495, NP_001356220, NP_001171481 and NP_001171481. The CDC45 protein can also be found at Uniprot #: 075419. Any of the sequences described herein can be used for expression of CDC45 as part of a molecule of the invention or a method of the invention.

In some embodiments, the open reading frame encodes Centrosomal protein of 295 kDa (CEP295) protein. In some embodiments, CEP295 protein comprises or consists of the amino acid sequence MKRKVVNTHKLRLSPNEEAFILKEDYERRRKLRLLQVREQERDIALQIREDIKQRR NQQFTRLAEELRAEWEESQTQKIQNLEKLYLASLRSMGEGHRQAKENEPDLDALA QRAAERKRKADLRHKEALKVQKNQKEILLKQKTWHIKARKEALLVEKERSAKITS LPPPPPTLFENIEVKRISAVKTNSSTYHHLHTFVNRETDTKRPDARLAAEEEAKRLE

(SEQ ID NO: 12)
MKRKVVNTHKLRLSPNEEAFILKEDYERRRKLRLLQVREQERDIALQIREDIKQRR
NQQFTRLAEELRAEWEESQTQKIQNLEKLYLASLRSMGEGHRQAKENEPDLDALA
QRAAERKRKADLRHKEALKVQKNQKEILLKQKTWHIKARKEALLVEKERSAKITS
LPPPPPTLFENIEVKRISAVKTNSSTYHHLHTFVNRETDTKRPDARLAAEEEAKRLE
ELQKQAAQERMERFEKAHVRGFQAMKKIHLAQNQEKLMKELKQLQQEDLARRR
QTVAQMPPQLVELPYKRSEMKEDWQRELEFAFEDMYNADRKVKGNLILHLEPEP
LPTVTNQIQDEELDLSMEQENLGAAEDLPVTEAEICSSETDVPLVMKTQQIPSKVLF
KKLLNKIRSQKSLWTIKSMSEDESEMITTVSEIESKAPTVESGTIASKERTLSSGQEQ
VVESDTLTIESGPLASEDKPLSCGTNSGKEQEINETLPITTVAQSSVLLHPQEAAARI
RMSARQKQIMEIEEQKQKQLELLEQIEQQKLRLETDCFRAQLEEEKRKKTQPTGV
GIAPASCPVISDEDSHRQMIRNYQHQLLQQNRLHRQSVETARKQLLEYQTMLKGR
CPSVSAPSLITDSVISVPSWKSERPTAISEHWDQGQRLKLSPNKYQPIQPIQTSKLEQ
DHFQVARQNHFPQRQVETTETLRASDILTNQALESQEHLRQFSQTETQQRDYKLV
PKDSETLSRALSHDRQLISQDARKISETFGATTFQSLESQQLFSENSENISYHLTEPSS
FVPLVPQHSFSSLPVKVESGKIQEPFSAMSKSTVSTSHSIISQMHDRPLLPSENITAQ
QGNMKALQEQLDLQKKVLQATQEAQEQLLLCKQKEVEQQTGLSVFLPLVTPDSS
ALLPSAKADLGRIQESSPTKNNIAVSSDHHVISQLQDKRLSLSQPILSQQNNFKFLQ
EQLNIQKDSLQARREAQEVLYVHKQSELDRRVCSEQAEPSFPFQVAQHTFTSLPSA
DTKSGKIQEQHSSKSEKGLVSCQSDIPISQDGSLSFLQQFLPLHDSLKLLQEQLTKQ
RDTLQARHEAQVELLLHRQRDLGDSKSGLVSSSSSPVVVQHSVASQASAKAEPRRI
QELYLSEKENVGPSCHLIIPTFQDKSLSFPQHSLAQQENLTILQEQSQIQRVILGAKE
GTQEFVHTESELEKRISSEQTGTSSSLSQVDESERFQECISIKSDSTIPLSHPKIPRCQE
RLLRVSQHMLPLQDNLEEHQAWLDTEKEAFHFSQKTQENTSSEQTGSSSFIPQLVQ
LSFTSLASAESGTILEPLFTESESKIFSSHLQIPQLQDRLLRISQLIQPQQDNLKALQE
QLATQREAIILARQEAREELLLHQSEWEGRISPEQVDTSSLPLVPQHSFASLPLNESE
RNQEPCSINSDNIVSSGHSEIPTLPDGLLGLSHLVLPQQDNLIALEEHLHAQTDFLPSI
EKTQKELVLSKPCKFEEKVSSEHFIQSHHGDLQALQQQLDTQKKAIRSIQEVQEEL
LLQRLSELEKRVSSEQVCSSSFVSQVPVADSERTQKSFPTKSNDTLPSSHREIPRLQD
RLLSLSKPILPQQDNMTAQLDAQREVMYSYEKPQEELSLNKQRKLNKSESAEHTIP
SLFLPKETEHSFIPLPFAEAKPKSTCELYSSQNEHAAPPSNPVIPGFQDRLLSFSQSVL
TQQDNLGLQKQLDLQREVLHYSQKAQEKLLVQRQTALQQQIQKHEETLKDFFKD
SQISKPTVENDLKTQKMGQLRDWFPNTQDLAGNDQENIRHADRNNSDDNHLASE
DTSAKQSGEHLEKDLGRRSSKPPVAKVKCGLDLNQHELSAIQEVESPAIGRTSILG
KPGIYEDRDPLRVSISREQSFFGSPLAHDPFSCLQLVGQENVCGDDYDEAVKLKES
VVENHAVLSYAVEEEHAYLGPTVKPDDKAKTLSYEPLSSATVSTGSLLSYENTDLS
LTDPESFSEHMDDSKQESTTSKEEETNIISSIVPSTQDIYQRQNSSDVHKSLLPAVDE
TTCGHTHFQQMIDKYINEANLIPEKTDLQELEHIFPNLHHQLFKPLEPHPDFDLSSSS
SGISPDNRDFYQRSDSSSESHCATGLSKSTVYFTALRRTSMHSSLNTSPNQQPDTNL
AHVGAHSFATENIIGGSEQCFEQLQPEYSSQEESQHADLPSIFSIEARDSSQGMKNQ
NYPSEEHTEILQNKKKIVHFQLSIGNLSSVYSSSDEANVFDQLNVQHSTPCGSNSSE
CSTKHQLESRKESMGFEELSKRGVVTMLQSQGLIEDNKNETCRVLDINPQVEETDS
RLCVRTVEMGTSIQAPYSLTTQNEKYFENSAETDIPKITKKLSQLGESELFASSGSFS
LOSSIPVWETETGHGIMEEPELTLISTTDTSIAEMDFANLTLEEKSENEAKCFFQVSE
FLPLVSATEASDYPAVSELSIEKPRTASTETPRRLTPVPGSLQEAFIKRKKSFMERSH
QRQKEIRNKIHVSENSQIKTVKEKPSISSSVSRLKGVNKVRASFPEDRKTTQALRHQ
RGLRLYNQLAEVKQQKEEKTKQEAYAQNRARAKEFHKKTLEKLRAKNTC.

ID NO: 12). In some embodiments, the CEP295 CDS comprises or consists of the nucleic acid sequence

(SEQ ID NO: 3)
atgaagagaaaagtcgtgaatactcacaagctgagattgagtcctaatgaggaagccttcattttgaaggaagattatgaaagaagg
cgaaaactaagattgctacaggttcgagaacaagaaagagatatcgccttacagataagagaagacataaaacagaggagaaat
caacaatttacacgtttggcagaggagctaagggcagaatgggaagaatcacaaactcagaaaatacagaacttggaaaaactgt
atttggcaagtttaagaagtatgggagagggacatcgacaggccaaagaaaatgaacctgatttggatgctttggcacagcgggc
agcagaaaggaaaagaaaagcagatttgaggcataaagaagccttgaaagtacagaaaaatcaaaaagaaatattactgaaaca
aaaaacctggcatataaaagctcgaaaggaagcactgcttgtggaaaaagagagatcagccaaaattacaagtctgccacctcct
cctccaactctttttgagaacatcgaagtaaaaagaatttctgcagtcaaaaccaatagttctacctaccatcatcttcacacttttgtga
atagagagacagacacaaaacggccagatgctcgtttggctgctgaagaggaagctaaacgattggaagaactacaaaaacagg
cagcacaagagagaatggaacggtttgaaaaggcacatgtacggggattccaagcaatgaagaagatccatttggctcaaaatca
ggagaaactaatgaaagaactcaaacagctacagcaagaggacctggcacgtaggagacagactgtagcacaaatgccaccac
aactagttgaacttccatacaaacgcagtgaaatgaaagaagactggcagagagaattggaatttgcctttgaagatatgtacaatg
cagacaggaaggtgaaagggaatctgattctgcaccttgaaccagagcccttgcccactgtgactaatcagatccaagatgaaga
gctggacctttcaatggaacaagaaaatttgggtgcagctgaagaccttccagtgacagaagctgaaatatgttctagtgaaacaga
tgttcccttggtaatgaagacccaacagattccttcaaaagttctttttaaaaaattattaaataagatccgaagccaaaaatctctctgg
acaattaaatctatgtctgaggatgaaagtgaaatgattacgactgttagtgaaattgagagtaaagcaccaacggttgagtcagga
acaattgccagcaaagagagaacgttatcctctgggcaggaacaagttgttgaaagtgatacactaacaattgagtctggaccactt
gctagtgaagataaaccactttcgtgtggtacaaactctggaaaagaacaagaaataaatgagactctgcctatcacaactgtagct
cagagttcagttctacttcatcctcaagaagcagcagccaggattagaatgtcagcaaggcagaaacagataatggaaatagaag
agcagaagcaaaagcaattggaattacttgaacaaattgaacagcagaaattaagattagaaactgactgcttcagggctcagctg
gaagaagaaaaaagaaaaaaaactcaaccgactggggttggcattgctccagcatcatgccctgtaatttctgatgaagatagtcat
aggcagatgattcgtaactatcaacatcagcttttacaacaaaacaggttacacaggcagtctgttgaaacagccaggaaacaatta
cttgaatatcaaactatgttaaaaggaaggtgcccatcggtgtcagctccatcattgataactgattctgttatatcagtgccatcatgg
aaatctgagagaccgactgctatatcagagcattgggatcaaggtcagagactcaagttgagtcctaacaaataccaacccataca
acctatacagacctccaaattagaacaagatcattttcaggtagcgagacaaaatcactttccacaaagacaggtggaaacaacag
aaacattacgcgcttcagatattttaaccaatcaagctttagaatcacaagaacatctaaggcaattctctcagactgaaacacaaca
gagagactataaattggtccccaaagattctgagacactttcaagggctttgtcacatgacaggcagctaatatcacaggatgctag
aaaaatatctgaaacatttggggcaacaacttttcaaagtttagaatcccaacaattgttctcagagaatagtgaaaatatatcttaccat
ttaactgaaccttcttcatttgtaccactggtacctcagcattcttttagttctctgcctgttaaagttgagtcaggaaaaattcaagaacc
cttttcagccatgagcaaaagtacagtttccacaagccattctataatcagccaaatgcatgataggcctttgctgccgtcagagaata
tcacagcccagcaaggtaatatgaaggccctccaagaacagttagacctacagaagaaagttcttcaggcaactcaggaagctca
ggaacagttgcttttgtgcaaacagaaagaagtggaacagcaaacgggcctctcggtattccttcccttggtaactccagattcatct
gctttattgccttctgccaaagcagatttggggagaatccaggaatcttcaccaaccaagaataatattgcagtttcctcagaccatca
tgtgatctcacaacttcaggataagcgtttgagtctttcacagcctatcctatcacagcaaaataattttaaatttctccaagagcagttg
aatattcagaaggatagccttcaggctaggcgagaagcccaggaagtattgtatgtacataaacagagtgaattggatagaagagt
atgttccgaacaggctgagccctctttcccatttcaggtagctcagcatacatttacttcactaccatctgctgatacaaaatctggaaa
aatacaggagcaacattcatctaagagcgagaaaggacttgtttcatgccaatctgacatccccatatctcaggatgggtctttgagtt
tcctacagcagttcctacctctacatgatagtttgaagttgctccaagaacagttgactaaacagagggatactcttcaggctaggcat
gaagctcaggtggaattacttttacatagacaaagagatttgggggacagtaagtctgggctggtgagctcttcatcctcaccagtg
gttgttcagcattcagttgcttcacaagcttctgctaaagctgagcctaggagaattcaggagctttatttatctgagaaggagaatgta
ggtccctcctgtcatttgataatcccaacatttcaggataagtctcttagttttccacagcatagcctggcacagcaagaaaatttgaca
atactccaagaacagtcacaaatacaaagggtaatacttggtgctaaagaaggaactcaggaatttgtacacacagaaagtgaatt
ggagaaaagaatttcttctgaacagactggcacctcctcatccctttcccaggtggatgaatctgagagattccaggaatgtatatca
atcaagagtgacagtaccattcccttaagccatcctaagatcccaagatgtcaggaaagacttttgagagtttcacaacatatgctac
ctctacaagataatttggaggaacaccaagcatggctagacactgagaaagaagcctttcatttcagccagaaaacccaagaaaat
acatcttctgaacaaactggttcatcttcattcataccccagttggtacagctttcatttacttcgttagcttcagctgagtctggcacaat
cctggaacctctttttacagagagtgaaagtaaaattttttcaagccaccttcagatcccacaattgcaggataggcttttgaggatatc
gcaacttatccagcctcaacaagataatttgaaggcacttcaagaacagttagctacacagagagaagccatcattctagctagaca
agaagctcgggaagaattacttttacatcagagtgaatgggagggaagaatatctcccgagcaggttgacacctcttccttacccct
agtaccacagcattcattcgcctcattacctcttaatgaatctgaaagaaaccaagaaccatgttcaattaacagtgataatatagtatc
ctcaggtcactcagagataccaacattgcctgatgggctgttgggtttatcacatcttgttttacctcaacaagataatttgattgcactt
gaagaacacttgcatgcacagacagatttccttccttctattgagaaaacccagaaagaattggttttgtcaaaaccatgtaaatttga
ggaaaaggtatcttctgagcattttatccagtctcaccatggtgatttgcaggcacttcaacagcagttagatacacagaagaaagcc
attcgatctatacaggaagtccaagaagaattgcttttgcaaagattaagtgaattggagaaaagggtatcatctgaacaagtttgctc
ctcttcatttgtatcccaggtgcctgttgctgactctgaaagaacccagaagtctttcccaaccaaaagtaatgatactcttccctcaag
tcatcgtgagattccaagattacaggatagacttttgagtttatcaaagcctattctgcctcagcaagataatatgacagcacaattgga
tgcacaaagggaagtgatgtattcttatgagaaaccccaggaagaactgtctttaaacaaacaaagaaagttgaacaaaagtgaat
ctgctgagcatactatcccctctttgtttctacccaaggaaacagagcattcgtttattccactaccttttgcagaagctaaacctaaaa
gcacttgtgaattgtattcatcccagaatgaacatgcagcccccccaagtaatcctgtgatcccagggtttcaagatagacttttgagt
ttttcacagtctgtcttaactcagcaagataacttgggacttcagaaacagttggatctacaaagagaagttctgcattatagccagaa
agcccaggaaaaattgcttgtacagagacaaacagcattgcagcagcagatacagaaacatgaagagactttgaaggatttcttta
aagacagtcagataagtaagcccacagttgaaaatgatttaaaaacccagaagatggggcagctcagagactggtttcctaataca
caagacctagcaggaaatgatcaagaaaatattaggcatgcagataggaacaactctgatgataatcatttggcttcagaagatact
agtgccaagcaaagtggtgagcatctggagaaagatctggggagaagatcctcaaagccacctgtagcaaaagtcaaatgtggtt
tggacttaaaccagcatgaacttagtgctatacaagaagtagagtcaccagcaattggcagaacttctatactaggtaaaccaggtat
ttatgaagacagagaccccctgcgagtctcaataagccgagaacaaagtttctttgggagcccactggcccatgatccgtttagttgt
cttcaactggttggccaagagaatgtctgtggtgatgactatgatgaagcagttaagctgaaggaatctgttgttgaaaatcatgcagt
gttaagttatgctgtggaggaagaacatgcatatttgggtccaactgtgaagccagatgataaggctaaaacactgtcttatgaacca
ttatcttcagcaactgtttccactgggagccttttaagttatgaaaacacagatttgagccttacagatccagagtcattttcagagcac
atggatgatagcaagcaagaatctaccaccagtaaagaagaggaaacaaatattataagttccatagttccttcaacacaagatattt
atcagcggcagaactcttcagacgttcataaatctctgttgcctgcagtggatgaaactacatgtggtcacacacactttcagcaaat
gatagacaagtacattaatgaagcaaatttgatacctgaaaaaacagatttgcaagaattggaacacatttttcctaatttgcatcatca
gctgtttaaacccttagaaccacatccagattttgacttatcatcatcatcctctgggatttctccagacaacagagacttttaccagag
atcagattcttcatctgaaagccactgtgctactggattatccaaaagtacagtttatttcacagcactgaggaggaccagcatgcatt
cttctcttaacacaagtccgaatcaacaacctgacactaacttggctcatgttggagctcacagttttgctacagaaaatattattgggg
gatctgaacaatgttttgaacagcttcagccagaatattcttcacaggaggagagccagcatgctgatctaccaagtatttttagcatt
gaagcaagagattcttcccaaggcatgaaaaatcagaactatccctctgaagaacatactgaaatattacaaaacaagaaaaaaatt
gttcatttccagctttctataggaaacttaagttcagtctacagttcatctgatgaagctaatgtatttgatcagttaaatgtacagcatag
cactccatgtggttctaactctagtgagtgctcaacaaaacaccaactagaaagcagaaaggaaagtatgggctttgaagaactatc
aaaaagaggggttgttacaatgttacaaagtcaaggactcattgaagataataaaaatgaaacctgtagggttttagacataaatcca
caggtagaggaaactgactctcgattatgtgtaagaacagtggagatgggaacttcaattcaggcaccatattccttaactactcaaa
atgaaaaatattttgagaattcagctgaaacagacattccaaaaatcaccaaaaaactatctcaactaggagaatcagagctttttgca
agttctggatcattttcattacagagctctataccagtctgggaaacagaaactggccatggtataatggaagaaccagaacttacttt
aataagcaccactgataccagtattgctgaaatggattttgcaaatttaaccctagaagagaagagcgagaatgaagcaaaatgctt
ctttcaggtgagtgagtttctgcctcttgtatcagcaacagaagcctcagattatccagctgtatcagaactttccatagaaaaaccaa
ggacagcatctacagaaacccctcgcaggcttacacctgtaccagggagcttacaagaagcatttataaagaggaaaaaatcattt
atggagagatcccaccagaggcagaaagaaataaggaataaaattcatgtctctgaaaattctcaaatcaaaacagttaaagagaa
accatctataagttcatctgtgagtcgtctaaagggcgtgaataaagtcagagcatcttttcctgaagacagaaagactacacaggct
ctaaggcaccaaaggggtctaaggttatacaatcaactagctgaagtgaaacaacaaaaggaagaaaaaaaaaacaagaagct
tatgcccaaaacagagcaagggcaaaagaattccataagaaaacactagagaaacttcgagccaaaaatacatgc.

NO: 3). Human CEP295 mRNA sequences can be found in RefSeq accession number NM_033395. Human CEP295 protein sequences can be found in RefSeq accession number NP_203753. CEP295 protein can also be found at Uniprot #: Q9COD2. Any of the sequences described herein can be used for expression of CEP295 as part of a molecule of the invention or a method of the invention.

In some embodiments, the open reading frame encodes Superoxide dismutase [Mn], mitochondrial (SOD2) protein. In some embodiments, SOD2 protein comprises or consists of the amino acid sequence MLSRAVCGTSRQLAPVLGYLGSRQKHSLPDLPYDYGALEPHINAQIMQLHHSKHH AAYVNNLNVTEEKYQEALAKGDVTAQIALQPALKFNGGGHINHSIFWTNLSPNGG GEPKGELLEAIKRDFGSFDKFKEKLTAASVGVQGSGWGWLGFNKERGHLQIAACP NQDPLQGTTGLIPLLGIDVWEHAYYLQYKNVRPDYLKAIWNVINWENVTERYMA CKK (SEQ ID NO: 13). In some embodiments, the SOD2 CDS comprises or consists of the nucleic acid sequence atgttgagccgggcagtgtgcggcaccagcaggcagctggctccggttttggggtatctgggctccaggcagaagcacagcctc cccgacctgccctacgactacggcgccctggaacctcacatcaacgcgcagatcatgcagctgcaccacagcaagcaccacgc ggcctacgtgaacaacctgaacgtcaccgaggagaagtaccaggaggcgttggccaagggagatgttacagcccagatagctct tcagcctgcactgaagttcaatggtggtggtcatatcaatcatagcattttctggacaaacctcagccctaacggtggtggagaaccc aaaggggagttgctggaagccatcaaacgtgactttggttcctttgacaagtttaaggagaagctgacggctgcatctgttggtgtcc aaggctcaggttggggttggcttggtttcaataaggaacggggacacttacaaattgctgcttgtccaaatcaggatccactgcaag gaacaacaggccttattccactgctggggattgatgtgtgggagcacgcttactaccttcagtataaaaatgtcaggcctgattatcta aaagctatttggaatgtaatcaactgggagaatgtaactgaaagatacatggcttgcaaaaag (SEQ ID NO: 4). Human SOD2 mRNA sequences can be found in RefSeq accession numbers: NM_000636, NM_001024465, NM_001024466, NM_001322814 and NM_001322815. Human SOD2 protein sequences can be found in RefSeq accession numbers: NP_000627, NP_001019636, NP_001019637, NP_001309743 and NP_001309744. The SOD2 protein can also be found at Uniprot #: P04179. Any of the sequences described herein can be used for expression of SOD2 as part of a molecule of the invention or a method of the invention.

In some embodiments, the open reading frame encodes NADH dehydrogenase [ubiquinone]1 alpha subcomplex assembly factor 3 (NDUFAF3) protein. In some embodiments, NDUFAF3 protein comprises or consists of the amino acid sequence MYIDSYNSRGFMINGNRVLGPCALLPHSVVQWNVGSHQDITEDSFSLFWLLEPRIE IVVVGTGDRTERLQSQVLQAMRQRGIAVEVQDTPNACATFNFLCHEGRVTGAALI PPPGGTSLTSLGQAAQ (SEQ ID NO: 14). In some embodiments, the NDUFAF3 CDS comprises or consists of the nucleic acid sequence atgtacatcgacagctacaacagccgcggcttcatgataaacggaaaccgcgtgctcggcccctgcgctctgctcccgcactcgg tggtgcagtggaacgtgggatcccaccaggacatcaccgaagacagcttttccctcttctggttgctggagccccggatagagatc gtggtggtggggactggagaccggaccgagaggctgcagtcccaggtgcttcaagccatgaggcagcggggcattgctgtgga agtgcaggacacgcccaatgcctgtgccaccttcaacttcctgtgtcatgaaggccgagtaactggagctgctctcatccctccacc aggagggacttcacttacatctttgggccaagctgctcaa (SEQ ID NO: 5). Human NDUFAF3 mRNA sequences can be found in RefSeq accession numbers: NM_199417, NM_199069, NM_199070, NM_199073 and NM_199074. Human NDUFAF3 protein sequences can be found in RefSeq accession numbers: NP_951032, NP_951033, NP_951047, and NP_951056. The NDUFAF3 protein can also be found at Uniprot #: Q9BU61. Any of the sequences described herein can be used for expression of NDUFAF3 as part of a molecule of the invention or a method of the invention.

In some embodiments, the open reading frame encodes Frataxin, mitochondrial (FXN) protein. In some embodiments, FXN protein comprises or consists of the amino acid sequence MWTLGRRAVAGLLASPSPAQAQTLTRVPRPAELAPLCGRRGLRTDIDATCTPRRA SSNQRGLNQIWNVKKQSVYLMNLRKSGTLGHPGSLDETTYERLAEETLDSLAEFF EDLADKPYTFEDYDVSFGSGVLTVKLGGDLGTYVINKQTPNKQIWLSSPSSGPKRY DWTGKNWVYSHDGVSLHELLAAELTKALKTKLDLSSLAYSGKDA (SEQ ID NO: 15). In some embodiments, FXN protein comprises or consists of the amino acid sequence MWTLGRRAVAGLLASPSPAQAQTLTRVPRPAELAPLCGRRGLRTDIDATCTPRRA SSNQRGLNQIWNVKKQSVYLMNLRKSGTLGHPGVVS (SEQ ID NO: 16). In some embodiments, the FXN CDS comprises or consists of the nucleic acid sequence atgtggactctcgggcgccgcgcagtagccggcctcctggcgtcacccagcccagcccaggcccagaccctcacccgggtccc gcggccggcagagttggccccactctgcggccgccgtggcctgcgcaccgacatcgatgcgacctgcacgccccgccgcgca agttcgaaccaacgtggcctcaaccagatttggaatgtcaaaaagcagagtgtctatttgatgaatttgaggaaatctggaactttgg gccacccaggctctctagatgagaccacctatgaaagactagcagaggaaacgctggactctttagcagagttttttgaagaccttg cagacaagccatacacgtttgaggactatgatgtctcctttgggagtggtgtcttaactgtcaaactgggtggagatctaggaaccta tgtgatcaacaagcagacgccaaacaagcaaatctggctatcttctccatccagtggacctaagcgttatgactggactgggaaaa actgggtgtactcccacgacggcgtgtccctccatgagctgctggccgcagagctcactaaagccttaaaaaccaaactggacttg tcttccttggcctattccggaaaagatgct (SEQ ID NO: 6). In some embodiments, the FXN CDS comprises or consists of the nucleic acid sequence atgtggactctcgggcgccgcgcagtagccggcctcctggcgtcacccagcccagcccaggcccagaccctcacccgggtccc gcggccggcagagttggccccactctgcggccgccgtggcctgcgcaccgacatcgatgcgacctgcacgccccgccgcgca agttcgaaccaacgtggcctcaaccagatttggaatgtcaaaaagcagagtgtctatttgatgaatttgaggaaatctggaactttgg gccacccaggagtggtgtct (SEQ ID NO: 7). Human FXN mRNA sequences can be found in RefSeq accession numbers: NM_181425, NM_000144, and NM_001161706. Human FXN protein sequences can be found in RefSeq accession numbers: NP_000135, and NP_852090. The FXN protein can also be found at Uniprot #: Q16595. Any of the sequences described herein can be used for expression of FXN as part of a molecule of the invention or a method of the invention.

In some embodiments, the open reading frame encodes Cytochrome b-cl complex subunit 2, mitochondrial (UQCRC2) protein. In some embodiments, UQCRC2 protein comprises or consists of the amino acid sequence MKLLTRAGSFSRFYSLKVAPKVKATAAPAGAPPQPQDLEFTKLPNGLVIASLENYS PVSRIGLFIKAGSRYEDFSNLGTTHLLRLTSSLTTKGASSFKITRGIEAVGGKLSVTA TRENMAYTVECLRGDVDILMEFLLNVTTAPEFRRWEVADLQPQLKIDKAVAFQNP QTHVIENLHAAAYRNALANPLYCPDYRIGKVTSEELHYFVQNHFTSARMALIGLG VSHPVLKQVAEQFLNMRGGLGLSGAKANYRGGEIREQNGDSLVHAAFVAESAVA GSAEANAFSVLQHVLGAGPHVKRGSNTTSHLHQAVAKATQQPFDVSAFNASYSD SGLFGIYTISQATAAGDVIKAAYNQVKTIAQGNLSNTDVQAAKNKLKAGYLMSVE SSECFLEEVGSQALVAGSYMPPSTVLQQIDSVANADIINAAKKFVSGQKSMAASGN LGHTPFVDEL (SEQ ID NO: 17). In some embodiments, the UQCRC2 CDS comprises or consists of the nucleic acid sequence atgaagctactaaccagagccggctctttctcgagattttattccctcaaagttgcccccaaagttaaagccacagctgcgcctgcag gagcaccgccacaacctcaggaccttgagtttaccaagttaccaaatggcttggtgattgcttctttggaaaactattctcctgtatcaa gaattggtttgttcattaaagcaggcagtagatatgaggacttcagcaatttaggaaccacccatttgctgcgtcttacatccagtctga cgacaaaaggagcttcatctttcaagataacccgtggaattgaagcagttggtggcaaattaagtgtgaccgcaacaagggaaaac atggcttatactgtggaatgcctgcggggtgatgttgatattctaatggagttcctgctcaatgtcaccacagcaccagaatttcgtcgt tgggaagtagctgaccttcagcctcagctaaagattgacaaagctgtggcctttcagaatccgcagactcatgtcattgaaaatttgc atgcagcagcttaccggaatgccttggctaatcccttgtattgtcctgactataggattggaaaagtgacatcagaggagttacattac ttcgttcagaaccatttcacaagtgcaagaatggctttgattggacttggtgtgagtcatcctgttctaaagcaagttgctgaacagtttc tcaacatgaggggtgggcttggtttatctggtgcaaaggccaactaccgtggaggtgaaatccgagaacagaatggagacagtctt gtccatgctgcttttgtagcagaaagtgctgtcgcgggaagtgcagaggcaaatgcatttagtgttcttcagcatgtcctcggtgctg ggccacatgtcaagaggggcagcaacaccaccagccatctgcaccaggctgttgccaaggcaactcagcagccatttgatgtttc tgcatttaatgccagttactcagattctggactctttgggatttatactatctcccaggccacagctgctggagatgttatcaaggctgc ctataatcaagtaaaaacaatagctcaaggaaacctttccaacacagatgtccaagctgccaagaacaagctgaaagctggatacc taatgtcagtggagtcttctgagtgtttcctggaagaagtcgggtcccaggctctagttgctggttcttacatgccaccatccacagtc cttcagcagattgattcagtggctaatgctgatatcataaatgcggcaaagaagtttgtttctggccagaagtcaatggcagcaagtg gaaatttgggacatacaccttttgttgatgagttg (SEQ ID NO: 8). Human UQCRC2 mRNA sequences can be found in RefSeq accession number NM_003366. Human UQCRC2 protein sequences can be found in RefSeq accession number NP_003357. The UQCRC2 protein can also be found at Uniprot #: P22695. Any of the sequences described herein can be used for expression of UQCRC2 as part of a molecule of the invention or a method of the invention.

In some embodiments, the open reading frame comprises a variant nucleotide sequence with at least 70, 75, 80, 85, 90, 92, 95, 97, or 99% homology or identity to SEQ ID NO: 1. Each possibility represents a separate embodiment of the invention. In some embodiments, the open reading frame comprises a variant nucleotide sequence with at least 70, 75, 80, 85, 90, 92, 95, 97, or 99% homology or identity to SEQ ID NO: 2. Each possibility represents a separate embodiment of the invention. In some embodiments, the open reading frame comprises a variant nucleotide sequence with at least 70, 75, 80, 85, 90, 92, 95, 97, or 99% homology or identity to SEQ ID NO: 3. Each possibility represents a separate embodiment of the invention. In some embodiments, the open reading frame comprises a variant nucleotide sequence with at least 70, 75, 80, 85, 90, 92, 95, 97, or 99% homology or identity to SEQ ID NO: 4. Each possibility represents a separate embodiment of the invention. In some embodiments, the open reading frame comprises a variant nucleotide sequence with at least 70, 75, 80, 85, 90, 92, 95, 97, or 99% homology or identity to SEQ ID NO: 5. Each possibility represents a separate embodiment of the invention. In some embodiments, the open reading frame comprises a variant nucleotide sequence with at least 70, 75, 80, 85, 90, 92, 95, 97, or 99% homology or identity to SEQ ID NO: 6. Each possibility represents a separate embodiment of the invention. In some embodiments, the open reading frame comprises a variant nucleotide sequence with at least 70, 75, 80, 85, 90, 92, 95, 97, or 99% homology or identity to SEQ ID NO: 7. Each possibility represents a separate embodiment of the invention. In some embodiments, the open reading frame comprises a variant nucleotide sequence with at least 70, 75, 80, 85, 90, 92, 95, 97, or 99% homology or identity to SEQ ID NO: 8. Each possibility represents a separate embodiment of the invention. In some embodiments, the homology or identity is at least 80%. In some embodiments, the homology or identity is at least 85%. In some embodiments, the homology or identity is at least 90%. In some embodiments, the homology or identity is at least 95%.

In some embodiments, the open reading frame encodes a variant protein with at least 70, 75, 80, 85, 90, 92, 95, 97, or 99% homology or identity to SEQ ID NO: 9. Each possibility represents a separate embodiment of the invention. In some embodiments, the open reading frame encodes a variant protein with at least 70, 75, 80, 85, 90, 92, 95, 97, or 99% homology or identity to SEQ ID NO: 10. Each possibility represents a separate embodiment of the invention. In some embodiments, the open reading frame encodes a variant protein with at least 70, 75, 80, 85, 90, 92, 95, 97, or 99% homology or identity to SEQ ID NO: 11. Each possibility represents a separate embodiment of the invention. In some embodiments, the open reading frame encodes a variant protein with at least 70, 75, 80, 85, 90, 92, 95, 97, or 99% homology or identity to SEQ ID NO: 12. Each possibility represents a separate embodiment of the invention. In some embodiments, the open reading frame encodes a variant protein with at least 70, 75, 80, 85, 90, 92, 95, 97, or 99% homology or identity to SEQ ID NO: 13. Each possibility represents a separate embodiment of the invention. In some embodiments, the open reading frame encodes a variant protein with at least 70, 75, 80, 85, 90, 92, 95, 97, or 99% homology or identity to SEQ ID NO: 14. Each possibility represents a separate embodiment of the invention. In some embodiments, the open reading frame encodes a variant protein with at least 70, 75, 80, 85, 90, 92, 95, 97, or 99% homology or identity to SEQ ID NO: 15. Each possibility represents a separate embodiment of the invention. In some embodiments, the open reading frame encodes a variant protein with at least 70, 75, 80, 85, 90, 92, 95, 97, or 99% homology or identity to SEQ ID NO: 16. Each possibility represents a separate embodiment of the invention. In some embodiments, the open reading frame encodes a variant protein with at least 70, 75, 80, 85, 90, 92, 95, 97, or 99% homology or identity to SEQ ID NO: 17. Each possibility represents a separate embodiment of the invention. In some embodiments, the homology or identity is at least 80%. In some embodiments, the homology or identity is at least 85%. In some embodiments, the homology or identity is at least 90%. In some embodiments, the homology or identity is at least 95%.

In some embodiments, the protein is a variant protein with at least 70, 75, 80, 85, 90, 92, 95, 97, or 99% homology or identity to SEQ ID NO: 9. Each possibility represents a separate embodiment of the invention. In some embodiments, t the protein is a variant protein with at least 70, 75, 80, 85, 90, 92, 95, 97, or 99% homology or identity to SEQ ID NO: 10. Each possibility represents a separate embodiment of the invention. In some embodiments, the protein is a variant protein with at least 70, 75, 80, 85, 90, 92, 95, 97, or 99% homology or identity to SEQ ID NO: 11. Each possibility represents a separate embodiment of the invention. In some embodiments, the protein is a variant protein with at least 70, 75, 80, 85, 90, 92, 95, 97, or 99% homology or identity to SEQ ID NO: 12. Each possibility represents a separate embodiment of the invention. In some embodiments, the protein is a variant protein with at least 70, 75, 80, 85, 90, 92, 95, 97, or 99% homology or identity to SEQ ID NO: 13. Each possibility represents a separate embodiment of the invention. In some embodiments, the protein is a variant protein with at least 70, 75, 80, 85, 90, 92, 95, 97, or 99% homology or identity to SEQ ID NO: 14. Each possibility represents a separate embodiment of the invention. In some embodiments, the protein is a variant protein with at least 70, 75, 80, 85, 90, 92, 95, 97, or 99% homology or identity to SEQ ID NO: 15. Each possibility represents a separate embodiment of the invention. In some embodiments, the protein is a variant protein with at least 70, 75, 80, 85, 90, 92, 95, 97, or 99% homology or identity to SEQ ID NO: 16. Each possibility represents a separate embodiment of the invention. In some embodiments, the protein is a variant protein with at least 70, 75, 80, 85, 90, 92, 95, 97, or 99% homology or identity to SEQ ID NO: 17. Each possibility represents a separate embodiment of the invention. In some embodiments, the homology or identity is at least 80%. In some embodiments, the homology or identity is at least 85%. In some embodiments, the homology or identity is at least 90%. In some embodiments, the homology or identity is at least 95%.

In some embodiments, a variant retains DNA repair pathway functionality. In some embodiments, a variant retains unfolded protein response (UPR) functionality. In some embodiments, the UPR functionality is within mitochondria. In some embodiments, the variant retains mitochondrial stress reduction functionality. In some embodiments, the variant is still functional to be used in a method of the invention.

In some embodiments, the variant is a codon optimized variant. In some embodiments, the variant is optimized to have an increased GC content. In some embodiments, codon optimized is optimization of codon adaptation index (CAI). In some embodiments, optimized CAI is increased CAI. In some embodiments, increased is above a predetermined threshold. In some embodiments, the predetermined threshold is the level in the wildtype human sequence. In some embodiments, the predetermined threshold is an increase of at least 5%. In some embodiments, the variant is optimized for viral expression. In some embodiments, the variant is optimized for expression in insect cells. In some embodiments, the variant is optimized to remove cryptic splice sites. In some embodiments, the variant is optimized to remove common mutation sites. In some embodiments, the variant is optimized to remove error-prone sites. In some embodiments, the variant comprises only synonymous mutations. In some embodiments, the variant still encodes the same protein without any amino acid changes.

In some embodiments, the UBL5 coding sequence comprises the nucleotide sequence atgatcgaggtggtgtgcaacgataggctgggcaagaaggtgagggtgaagtgcaataccgatgacaccatcggcgacctgaa gaagctgatcgccgcccagacaggaacacgctggaataaaatcgtgctgaagaagtggtacaccatcttcaaggatcacgtgtct ctgggcgactatgagatccacgacggcatgaatctggagctgtactatcag (SEQ ID NO: 18). In some embodiments, the UBL5 coding sequence consists of SEQ ID NO: 18. In some embodiments, the open reading frame consists of SEQ ID NO: 18. In some embodiments, an optimized UBL5 coding sequence comprises or consists of SEQ ID NO: 18. In some embodiments, the UBL5 coding sequence comprises or consists of a variant sequence with at least 70, 75, 80, 85, 90, 92, 95, 97, or 99% identity or homology to SEQ ID NO: 18. Each possibility represents a separate embodiment of the invention. In some embodiments, the homology or identity is at least 80%. In some embodiments, the homology or identity is at least 85%. In some embodiments, the homology or identity is at least 90%. In some embodiments, the homology or identity is at least 95%. It will be evident to a skilled artisan that SEQ ID NO: 18 contains only synonymous codon mutations and therefore still encodes SEQ ID NO: 9. In some embodiments, a variant of SEQ ID NO: 18 encodes SEQ ID NO: 9.

In some embodiments, the nanoparticle comprises a shell. In some embodiments, the shell is an outer shell. In some embodiments, the nanoparticle comprises a core. In some embodiments, the core is within the shell. In some embodiments, the shell surrounds the core. In some embodiments, the core is an aqueous core. In some embodiments, the core comprises the nucleic acid molecule. In some embodiments, the core comprises the protein. In some embodiments, the protein is embedded in the shell. In some embodiments, the protein is attached to the shell. In some embodiments, attached is conjugated to.

As used herein, the term “nanoparticle” refers to a particle having an average size of up to about 1000 nm, as determined by any method known in the art, for example dynamic light scattering (DLS) for determining the hydrodynamic diameter of the particles and transmission electron microscopy (TEM) for determining the accurate geometric nanoparticle size. According to some embodiments, the size of the nanoparticle is within the range of 50-1000, 100-1000, 200-1000, 250-1000, 300-1000, 500-1000, 600-1000, 700-1000, 50-900, 100-900, 200-900, 250-900, 300-900, 500-900, 600-900, 700-900, 50-800, 100-800, 200-800, 250-800, 300-800, 500-800, 600-800, 700-800, 50-600, 100-600, 200-600, 250-600, 300-600, 500-600, or 50-200 nm. Each possibility represents a separate embodiment of the present invention. In some embodiments, the nanoparticle is a synthetic nanoparticle. In some embodiments, the nanoparticle is a viral nanoparticle. In some embodiments, the nanoparticle is a lipid nanoparticle. Lipid-based nanoparticles include liposomes, solid lipid nanoparticles, nanostructured lipid carriers, metal-organic frameworks, and polymeric nanoparticles such as PLGA, chitosan, polyethylenimine, poly(beta-amino esters) nanoparticles. In some embodiments, a lipid nanoparticle is a liposome. In some embodiments, a lipid nanoparticle is a micelle. In some embodiments, the nanoparticle is a polymeric nanoparticle. In some embodiments, the nanoparticle is a synthetic nanoparticle. In some embodiments, a synthetic nanoparticle is an inorganic nanoparticle. Inorganic nanoparticles include gold nanoparticles, silica nanoparticles, quantum dots, iron oxide nanoparticles, silver nanoparticles, dendrimers, carbon-based nanoparticles including carbon nanotubes, graphene oxide, fullerenes, magnetic nanoparticles, and mesoporous silica nanoparticles. In some embodiments, the nanoparticle is a metallic nanoparticle. In some embodiments, the nanoparticle is a viral particle. In some embodiments, the virus is a lentivirus. In some embodiments, the virus is an adeno-associated virus (AAV). In some embodiments, the nanoparticle is an AAV nanoparticle. In some embodiments, the shell is a capsid shell.

AAV serotypes are well known in the art and any AAV may be used. Examples of AAV serotypes include, but are not limited to AAV1, AAV2, AAV3, AAV4, AAV5, AAV6, AAV6.2, AAV7, AAV8, and AAV9. In some embodiments, the AAV is AAV9. In some embodiments, the AAV9 is AAV-DJ. In some embodiments, the AAV is a recombinant AAV (rAAV). In some embodiments, the AAV9 is rAAV9. In some embodiments, the AAV is selected based on its targeting to a target cell. In some embodiments, a target cell is a cell of a target tissue. AAV targeting is well known in the art and information on tissue tropism can be found for example at en.vectorbuilder.com/products-services/service/aav-packaging.html.

In some embodiments, the nanoparticle is targeted to immune cells. In some embodiments, the immune cells are primary immune cells. In some embodiments, the immune cells are CD44 positive immune cells. In some embodiments, the immune cells are bone marrow immune cells. In some embodiments, the immune cells are activated immune cells. In some embodiments, the immune cells are lymphocytes. In some embodiments, the immune cells are T cells. In some embodiments, the nanoparticle comprises a targeting peptide. In some embodiments, the targeting peptide is to immune cells. In some embodiments, the targeting peptide is to CD44. In some embodiments, the nanoparticle comprises a CD44 targeting peptide. In some embodiments, the peptide is on the shell. In some embodiments, the targeting peptide is in the shell. In some embodiments, the targeting peptide is on the outside of the shell. It will be understood that a surface facing to the core of the nanoparticle will be viewed as the inside and the surface facing toward everything else is facing to the outside. In some embodiments, the targeting peptide faces to the outside of the nanoparticle.

In some embodiments, the CD44 targeting peptide is elected from the group consisting of: YNGTIFF (SEQ ID NO: 19), RSIFFLK (SEQ ID NO: 20), LVSYFGI (SEQ ID NO: 21), NPIIFFL (SEQ ID NO: 22), YNGIIVF (SEQ ID NO: 23), LVPYNHI (SEQ ID NO: 24), LVSYNGM (SEQ ID NO: 25), VSYHGII (SEQ ID NO: 26), YNGIMFF (SEQ ID NO: 27), YNGIILF (SEQ ID NO: 28) and GIQFFTK (SEQ ID NO: 29). In some embodiments, the CD44 targeting peptide is SEQ ID NO: 19. In some embodiments, the CD44 targeting peptide is SEQ ID NO: 20. In some embodiments, the CD44 targeting peptide is SEQ ID NO: 21. In some embodiments, the CD44 targeting peptide is SEQ ID NO: 22. In some embodiments, the CD44 targeting peptide is SEQ ID NO: 23. In some embodiments, the CD44 targeting peptide is SEQ ID NO: 24. In some embodiments, the CD44 targeting peptide is SEQ ID NO: 25. In some embodiments, the CD44 targeting peptide is SEQ ID NO: 26. In some embodiments, the CD44 targeting peptide is SEQ ID NO: 27. In some embodiments, the CD44 targeting peptide is SEQ ID NO: 28. In some embodiments, the CD44 targeting peptide is SEQ ID NO: 29.

In some embodiments, the targeting peptide is inserted into a capsid of the viral nanoparticle. In some embodiments, the capsid is a fusion protein comprising the targeting peptide. In some embodiments, the targeting peptide is integrated into the amino acid sequence of the capsid. In some embodiments, the capsid is a fusion protein of the invention. In some embodiments, the targeting peptide is inserted into the coat protein domain of the capsid. In some embodiments, the targeting peptide is not inserted into a phospholipase-like domain. In some embodiments, phospholipase is phospholipase A2. In some embodiments, the targeting peptide is not inserted into a disordered region. In some embodiments, the targeting peptide is integrated into a sequence of the capsid that faces outwards. In some embodiments, the targeting peptide is integrated into a sequence on the outside of the capsid. In some embodiments, the targeting peptide does not replace any of the sequence of the capsid.

In some embodiments, the capsid is the AAV9 capsid. In some embodiments, the capsid is capsid protein VPL. In some embodiments, the AAV9 capsid comprises or consists of the amino acid sequence MAADGYLPDWLEDNLSEGIREWWALKPGAPQPKANQQHQDNARGLVLPGYKYL GPGNGLDKGEPVNAADAAALEHDKAYDQQLKAGDNPYLKYNHADAEFQERLKE DTSFGGNLGRAVFQAKKRLLEPLGLVEEAAKTAPGKKRPVEQSPQEPDSSAGIGKS GAQPAKKRLNFGQTGDTESVPDPQPIGEPPAAPSGVGSLTMASGGGAPVADNNEG ADGVGSSSGNWHCDSQWLGDRVITTSTRTWALPTYNNHLYKQISNSTSGGSSNDN AYFGYSTPWGYFDFNRFHCHFSPRDWQRLINNNWGFRPKRLNFKLFNIQVKEVTD NNGVKTIANNLTSTVQVFTDSDYQLPYVLGSAHEGCLPPFPADVFMIPQYGYLTLN DGSQAVGRSSFYCLEYFPSQMLRTGNNFQFSYEFENVPFHSSYAHSQSLDRLMNPL IDQYLYYLSKTINGSGQNQQTLKFSVAGPSNMAVQGRNYIPGPSYRQQRVSTTVT QNNNSEFAWPGASSWALNGRNSLMNPGPAMASHKEGEDRFFPLSGSLIFGKQGTG RDNVDADKVMITNEEEIKTTNPVATESYGQVATNHQSAQAQAQTGWVQNQGILP GMVWQDRDVYLQGPIWAKIPHTDGNFHPSPLMGGFGMKHPPPQILIKNTPVPADP PTAFNKDKLNSFITQYSTGQVSVEIEWELQKENSKRWNPEIQYTSNYYKSNNVEFA VNTEGVYSEPRPIGTRYLTRNL (SEQ ID NO: 30). In some embodiments, the targeting peptide is inserted between glutamine 588 and alanine 589 of the capsid. In some embodiments, the targeting peptide is inserted between glutamine 588 and alanine 589 of SEQ ID NO: 30. In some embodiments, glutamine 588 and alanine 589 are with respect to SEQ ID NO: 30. In some embodiments, the targeting peptide is inserted between residues equivalent to glutamine 588 and alanine 589 in another capsid. In some embodiments, the capsid fusion protein comprising the targeting peptide is selected from the group consisting of: SEQ ID NO: 31-41. In some embodiments, the capsid fusion protein comprising the targeting peptide comprises or consists of an amino acid sequence selected from the group consisting of: SEQ ID NO: 31-41. In some embodiments, the capsid fusion protein comprising the targeting peptide comprises the amino acid sequence of SEQ ID NO: 31. In some embodiments, the capsid fusion protein comprising the targeting peptide consists of the amino acid sequence of SEQ ID NO: 31.

By another aspect, there is provided a nanoparticle comprises a peptide of the invention. By another aspect, there is provided a nanoparticle comprises a fusion protein of the invention. By another aspect, there is provided a nanoparticle comprises a nucleic acid molecule of the invention. By another aspect, there is provided a nanoparticle comprises a vector of the invention.

### Targeting Peptides

By another aspect, there is provided a peptide comprising an amino acid sequence selected from the group consisting of: YNGTIFF (SEQ ID NO: 19), RSIFFLK (SEQ ID NO: 20), LVSYFGI (SEQ ID NO: 21), NPIIFFL (SEQ ID NO: 22), YNGIIVF (SEQ ID NO: 23), LVPYNHI (SEQ ID NO: 24), LVSYNGM (SEQ ID NO: 25), VSYHGII (SEQ ID NO: 26), YNGIMFF (SEQ ID NO: 27), YNGIILF (SEQ ID NO: 28) and GIQFFTK (SEQ ID NO: 29).

In some embodiments, the peptide consists of an amino acid sequence selected from SEQ ID NO: 19-29. In some embodiments, the peptide comprises SEQ ID NO: 19. In some embodiments, the peptide comprises SEQ ID NO: 20. In some embodiments, the peptide comprises SEQ ID NO: 21. In some embodiments, the peptide comprises SEQ ID NO: 22. In some embodiments, the peptide comprises SEQ ID NO: 23. In some embodiments, the peptide comprises SEQ ID NO: 24. In some embodiments, the peptide comprises SEQ ID NO: 25. In some embodiments, the peptide comprises SEQ ID NO: 26. In some embodiments, the peptide comprises SEQ ID NO: 27. In some embodiments, the peptide comprises SEQ ID NO: 28. In some embodiments, the peptide comprises SEQ ID NO: 29. In some embodiments, the peptide consists of SEQ ID NO: 19. In some embodiments, the peptide consists of SEQ ID NO: 20. In some embodiments, the peptide consists of SEQ ID NO: 21. In some embodiments, the peptide consists of SEQ ID NO: 22. In some embodiments, the peptide consists of SEQ ID NO: 23. In some embodiments, the peptide consists of SEQ ID NO: 24. In some embodiments, the peptide consists of SEQ ID NO: 25. In some embodiments, the peptide consists of SEQ ID NO: 26. In some embodiments, the peptide consists of SEQ ID NO: 27. In some embodiments, the peptide consists of SEQ ID NO: 28. In some embodiments, the peptide consists of SEQ ID NO: 29.

In some embodiments, the peptide comprises between 7 and 35000, 7 and 30000, 7 and 25000, 7 and 20000, 7 and 15000, 7 and 10000, 7 and 9000, 7 and 8000, 7 and 7000, 7 and 6000, 7 and 5000, 7 and 4000, 7 and 3000, 7 and 2000, 7 and 1000, 7 and 950, 7 and 900, 7 and 850, 7 and 800, 7 and 750, 7 and 700, 7 and 650, 7 and 600, 7 and 550, 7 and 500, 7 and 450, 7 and 400, 7 and 350, 7 and 300, 7 and 250, 7 and 200, 7 and 150, 7 and 100, 7 and 90, 7 and 80, 7 and 70, 7 and 60, 7 and 50, 7 and 45, 7 and 40, 7 and 35, 7 and 30, 7 and 25, 7 and 20, 7 and 15, and 7 and 10 amino acids. Each possibility represents a separate embodiment of the invention. In some embodiments, the peptide comprises between 7 and 30 amino acids. In some embodiments, the peptide comprises between 7 and 100 amino acids.

In some embodiments, the peptide is for use in targeting to CD44. In some embodiments, the peptide binds to CD44. In some embodiments, the peptide is for use in targeting to CD44 expressing cells. In some embodiments, CD44 is cell surface CD44. In some embodiments, the peptide binds to cells expressing CD44. In some embodiments, the peptide is a CD44 targeting peptide. In some embodiments, the peptide is for use in delivering an agent to a CD44 expressing cell. In some embodiments, the peptide and agent are in the same composition.

### Capsid Fusion Proteins

By another aspect, there is provided a fusion protein comprising an amino acid sequence of a viral capsid protein and an amino acid sequence of CD44 targeting peptide.

In some embodiments, the CD44 targeting peptide is a peptide of the invention. In some embodiments, the CD44 targeting peptide comprises an amino acid sequence selected from SEQ ID NO: 19-29. In some embodiments, the CD44 targeting peptide consists of an amino acid sequence selected from SEQ ID NO: 19-29. In some embodiments, fusion protein retains viral packaging function. In some embodiments, the fusion protein retains the ability to form a viral nanoparticle. It will be understood by the skilled artisan that the peptide will be inserted in the capsid such that it does not disrupt viral packaging and formation of a viral particle. Further, the peptide will be inserted such that it faces outward/is on the surface of a viral particle that is formed.

In some embodiments, the fusion protein is a capsid fusion protein. In some embodiments, the fusion protein is a targeting fusion protein. In some embodiments, the viral capsid is AAV capsid. In some embodiments, the capsid is capsid protein VP1. In some embodiments, the viral capsid is an AAV9 capsid. In some embodiments, the AAV9 capsid comprises an amino acid sequence of SEQ ID NO: 30. In some embodiments, the AAV9 capsid consists of an amino acid sequence of SEQ ID NO: 30. In some embodiments, the capsid comprises or consists of an amino acid sequence with at least 70, 75, 80, 85, 90, 95, 97 or 99% identity or homology to SEQ ID NO: 30. Each possibility represents a separate embodiment of the invention. In some embodiments, capsid retains viral packaging function. In some embodiments, the capsid retains the ability to form a viral nanoparticle.

In some embodiments, the targeting peptide is inserted between glutamine 588 and alanine 589 of the capsid. In some embodiments, the targeting peptide is inserted between glutamine 588 and alanine 589 of SEQ ID NO: 30. In some embodiments, glutamine 588 and alanine 589 are with respect to SEQ ID NO: 30. In some embodiments, the targeting peptide is inserted between residues equivalent to glutamine 588 and alanine 589 in another capsid.

In some embodiments, the fusion protein is selected from the group consisting of: SEQ ID NO: 31-41. In some embodiments, the fusion protein comprises an amino acid sequence selected from the group consisting of: SEQ ID NO: 31-41. In some embodiments, the fusion protein consists of an amino acid sequence selected from the group consisting of: SEQ ID NO: 31-41. In some embodiments, the fusion protein comprises or consists of an amino acid sequence with at least 70, 75, 80, 85, 90, 95, 97 or 99% identity or homology to a sequence selected from SEQ ID NO: 31-41. Each possibility represents a separate embodiment of the invention. In some embodiments, the homology or identity is at least 80%. In some embodiments, the homology or identity is at least 85%. In some embodiments, the homology or identity is at least 90%. In some embodiments, the homology or identity is at least 95%. In some embodiments, fusion protein retains viral packaging function. In some embodiments, the fusion protein retains the ability to form a viral nanoparticle. In some embodiments, the fusion protein comprises the amino acid sequence of SEQ ID NO: 31. In some embodiments, the fusion protein consists of the amino acid sequence of SEQ ID NO: 31.

### Nucleic Acid Molecules

By another aspect, there is provided a nucleic acid molecule comprising the nucleic acid sequence provided in SEQ ID NO: 18 or a variant sequence of SEQ ID NO: 18.

By another aspect, there is provided a nucleic acid molecule encoding a peptide of the invention.

By another aspect, there is provided a nucleic acid molecule encoding a fusion protein of the invention.

By another aspect, there is provided an expression vector comprising an open reading frame encoding at least one protein selected from UBL5, CDC45, CEP295, SOD2, NDUFAF3, FXN, and UQCRC2 wherein the open reading frame is operatively linked to a heterologous promoter configured to express in a target cell.

In some embodiments, the vector is selected from a lentiviral vector, an adenoviral vector, an adeno-associated viral vector, and a plasmid. In some embodiments, the vector comprises double stranded DNA, single stranded DNA, double stranded RNA, single stranded RNA, modified or non-standard nucleic acids or a combination thereof.

In some embodiments, a variant sequence comprises at least 70, 75, 80, 85, 90, 91, 95, 97, or 99% identity or homology to SEQ ID NO: 18. Each possibility represents a separate embodiment of the invention. Each possibility represents a separate embodiment of the invention. In some embodiments, the homology or identity is at least 80%. In some embodiments, the homology or identity is at least 85%. In some embodiments, the homology or identity is at least 90%. In some embodiments, the homology or identity is at least 95%. In some embodiments, the variant sequence comprises only synonymous codon substitutions within SEQ ID NO: 18. In some embodiments, the variant sequence encodes SEQ ID NO: 9.

In some embodiments, the nucleic acid molecule comprises an open reading frame. In some embodiments, the open reading frame comprises SEQ ID NO: 18 or the variant sequence. In some embodiments, the open reading frame encodes the peptide of the invention. In some embodiments, the open reading frame encodes the fusion protein of the invention. In some embodiments, nucleic acid molecule comprises a transcription regulatory element. In some embodiments, the transcription regulatory element is a promoter. In some embodiments, the open reading frame is operably linked to the transcriptional regulatory element. In some embodiments, the nucleic acid molecule is a vector.

A vector nucleic acid sequence generally contains at least an origin of replication for propagation in a cell and optionally additional elements, such as a heterologous polynucleotide sequence, expression control element (e.g., a promoter, enhancer), selectable marker (e.g., antibiotic resistance), poly-Adenine sequence.

The vector may be a DNA plasmid delivered via non-viral methods or via viral methods. The viral vector may be a retroviral vector, a herpesviral vector, an adenoviral vector, an adeno-associated viral vector or a poxviral vector. The promoters may be active in mammalian cells. The promoters may be viral promoters. In some embodiments, the nucleic acid molecule is a DNA molecule. In some embodiments, the nucleic acid molecule is an RNA molecule. In some embodiments, the vector is an AAV vector.

In some embodiments, the vector is introduced into the cell by standard methods including electroporation (e.g., as described in From et al., Proc. Natl. Acad. Sci. USA 82, 5824 (1985)), Heat shock, infection by viral vectors, high velocity ballistic penetration by small particles with the nucleic acid either within the matrix of small beads or particles, or on the surface (Klein et al., Nature 327. 70-73 (1987)), and/or the like.

In some embodiments, nucleic acid sequences are transcribed by RNA polymerase II (RNAP II and Pol II). RNAP II is an enzyme found in eukaryotic cells. It catalyzes the transcription of DNA to synthesize precursors of mRNA and most snRNA and microRNA.

In some embodiments, mammalian expression vectors include, but are not limited to, pcDNA3, pcDNA3.1 (±), pGL3, pZeoSV2(±), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

In some embodiments, expression vectors containing regulatory elements from eukaryotic viruses such as retroviruses are used by the present invention. SV40 vectors include pSVT7 and pMT2. In some embodiments, vectors derived from bovine papilloma virus include pBV-1MTHA, and vectors derived from Epstein Bar virus include pHEBO, and p2O5. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

In some embodiments, recombinant viral vectors, which offer advantages such as lateral infection and targeting specificity, are used for in vivo expression. In one embodiment, lateral infection is inherent in the life cycle of, for example, retrovirus and is the process by which a single infected cell produces many progeny virions that bud off and infect neighboring cells. In one embodiment, the result is that a large area becomes rapidly infected, most of which was not initially infected by the original viral particles. In one embodiment, viral vectors are produced that are unable to spread laterally. In one embodiment, this characteristic can be useful if the desired purpose is to introduce a specified gene into only a localized number of targeted cells.

Various methods can be used to introduce the expression vector of the present invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

It will be appreciated that other than containing the necessary elements for the transcription and translation of the inserted coding sequence (encoding the polypeptide), the expression construct of the present invention can also include sequences engineered to optimize stability, production, purification, yield or activity of the expressed polypeptide.

A person with skill in the art will appreciate that a gene can also be expressed from a nucleic acid construct administered to the individual (e.g., by a nanoparticle of the invention) employing any suitable mode of administration, described herein (i.e., in vivo gene therapy). In one embodiment, the nucleic acid construct is introduced into a suitable cell via an appropriate gene delivery vehicle/method (transfection, transduction, homologous recombination, the nanoparticle of the invention etc.) and an expression system as needed and then the modified cells are expanded in culture and returned to the individual (i.e., ex vivo gene therapy).

### Pharmaceutical Compositions

By another aspect, there is provided a composition comprising a nanoparticle of the invention.

By another aspect, there is provided a composition comprising a nucleic acid molecule of the invention.

By another aspect, there is provided a composition comprising a peptide of the invention.

By another aspect, there is provided a composition comprising a fusion protein of the invention.

In some embodiments, the composition is a pharmaceutical composition. In some embodiments, the composition is a therapeutic composition. In some embodiments, the composition is a diagnostic composition. In some embodiments, the composition comprises a pharmaceutically acceptable carrier, excipient or adjuvant. In some embodiments, the composition further comprises an agent.

As used herein, the term “carrier,” “excipient,” or “adjuvant” refers to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelies, insoluble monolayers, liquid crystals, phospholipid dispersions, lamellar layers and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein.

In some embodiments, the composition is formulated for administration to a subject. In some embodiments, the composition is formulated for systemic administration. In some embodiments, the composition is formulated for local administration to a disease site. In some embodiments, the composition is formulated for local administration to a target cell. In some embodiments, systemic administration is selected from intravenous, intramuscular, subcutaneous, and intraperitoneal administration.

As used herein, the terms “administering,” “administration,” and like terms refer to any method which, in sound medical practice, delivers a composition containing an active agent to a subject in such a manner as to provide a therapeutic effect. One aspect of the present subject matter provides for intravenous administration of a therapeutically effective amount of a composition of the present subject matter to a patient in need thereof. Other suitable routes of administration can include parenteral, subcutaneous, oral, intramuscular, or intraperitoneal.

The dosage administered will be dependent upon the age, health, and weight of the recipient, kind of concurrent treatment, if any, frequency of treatment, and the nature of the effect desired.

In some embodiments, the composition is for use in a method of the invention. In some embodiments, the composition is for use in a method of treatment of the invention. In some embodiments, the composition is for use in the production of a medicament for use in a method of treatment of the invention. In some embodiments, the composition is for use in treating a disease, disorder or condition characterized by mitochondrial stress. In some embodiments, the composition is for use in the production of a medicament for treating a disease, disorder or condition characterized by mitochondrial stress.

### Methods of Treatment

According to another aspect, there is provided a method of treating a disease, disorder or condition characterized by mitochondrial stress in a subject in need thereof, the method comprising increasing expression of a protein selected from UBL5, CDC45, CEP295, SOD2, NDUFAF3, FXN, and UQCRC2 in a diseased cell of the subject, thereby treating a disease characterized by mitochondrial stress.

According to another aspect, there is provided a method of treating a disease, disorder or condition characterized by mitochondrial stress in a subject in need thereof, the method comprising administering to the subject a composition of the invention, thereby treating a disease characterized by mitochondrial stress.

In some embodiments, the subject is a mammal. In some embodiments, the subject is a human. In some embodiments, the subject suffers from a disease, disorder or condition. In some embodiments, the disease, disorder or condition is further characterized by lipid stress. In some embodiments, the mitochondrial stress comprises abnormal lipid accumulation in disease cells or disease tissue of the subject. In some embodiments, abnormal is increased. In some embodiments, the lipid accumulation is intercellular lipid accumulation. In some embodiments, the lipid accumulation is intracellular lipid accumulation. In some embodiments, the abnormal lipid accumulation comprises the presence of lipid droplets in the diseased cells or diseased tissue at a level that is increased as compared to healthy cells or tissue. In some embodiments, increased is increased by at least 10, 15, 20, 25, 30, 35, 40, 45, 50, 55, 60, 65, 70, 75, 80, 85, 90, 95, 97, 99, 100, 150, 200, 250, 300, 350, 400, 450 or 500%. Each possibility represents a separate embodiment of the invention. In some embodiments, increased is significantly increased. In some embodiments, significantly is statistically significantly. In some embodiments, increased is increased by at least a predetermined threshold. In some embodiments, increased is increased to above a predetermined threshold. In some embodiments, the disease is characterized by lipid accumulation in disease cells or diseased tissue above a predetermined threshold.

In some embodiments, mitochondrial stress is reduced mitochondrial function. In some embodiments, mitochondrial stress comprises reduced mitochondrial function. In some embodiments, reduced is as compared to a control. In some embodiments, a control is a healthy control. In some embodiments, a control is control cells. In some embodiments, the control cells are of the same cell type as the disease cells. In some embodiments, the control cells are cell of a control tissue. In some embodiments, the control cells are healthy cells. In some embodiments, the control tissue is healthy tissue. In some embodiments, the control cells are of the same tissue as the disease cells.

In some embodiments, mitochondrial stress comprises an abnormal metabolic rate in disease cells or disease tissue. In some embodiments, the disease cells and/or diseased tissue are cells and/or tissue of the subject. In some embodiments, abnormal is decreased. In some embodiments, the metabolic rate is decreased. In some embodiments, the metabolic rate is the basal metabolic rate. Measuring metabolic rate is well known in the art and specific methods are disclosed herein. Any method of measuring metabolic rate may be employed. In some embodiments, the metabolic rate is decreased by at least a predetermined threshold. In some embodiments, the metabolic rate is below a predetermined threshold. In some embodiments, an abnormal metabolic rate comprises decreased oxygen consumption in the disease cells and/or diseased tissue. In some embodiments, decreased is reduced. In some embodiments, an abnormal metabolic rate comprises a decreased rate of oxygen consumption in the disease cells and/or diseased tissue. In some embodiments, decreased is decreased by at least 5, 10, 15, 20, 25, 30, 35, 40, 45, 50, 55, 60, 65, 70, 75, 80, 85, 90, 95, 97, 99, or 100%. Each possibility represents a separate embodiment of the invention. In some embodiments, decreased is significantly decreased. In some embodiments, significantly is statistically significantly. In some embodiments, decreased is decreased by at least a predetermined threshold. In some embodiments, decreased is decreased to below a predetermined threshold. In some embodiments, the disease is characterized by oxygen consumption or an oxygen consumption rate in disease cells or diseased tissue below a predetermined threshold. In some embodiments, the disease is characterized by a metabolic rate in disease cells or diseased tissue below a predetermined threshold.

In some embodiments, the disease, disorder or condition is selected from: a neuromuscular disease, an immune disease, a hematological disease and an ocular disease. In some embodiments, the disease, disorder or condition is selected from: a neuromuscular disease, an immune disease, a hematological disease, a cardiovascular disease, a neurodegenerative disease, a metabolic disorder or disease, a renal disorder, a dermatological condition, a cognitive disorder or a skeletomuscular condition. In some embodiments, the disease, disorder or condition is neuromuscular disease. In some embodiments, the disease, disorder or condition is an immune disease. In some embodiments, the disease, disorder or condition is a hematological disease. In some embodiments, the disease, disorder or condition is a cardiovascular disease. In some embodiments, the disease, disorder or condition is a neurodegenerative disease. In some embodiments, the disease, disorder or condition is metabolic disorder. In some embodiments, the disease, disorder or condition is metabolic disease. In some embodiments, the disease, disorder or condition is a renal disorder. In some embodiments, the disease, disorder or condition is a renal disease. In some embodiments, the disease, disorder or condition is dermatological condition. In some embodiments, the disease, disorder or condition is a dermatological disorder. In some embodiments, the disease, disorder or condition is a dermatological disease. In some embodiments, the disease, disorder or condition is a cognitive disorder. In some embodiments, the disease, disorder or condition is a cognitive disease. In some embodiments, the disease, disorder or condition is a skeletomuscular condition.

In some embodiments, the disease, disorder, or condition is selected from atherosclerosis, hyperlipidemia, hypercholesterolemia, hypertriglyceridemia, Alzheimer's disease, Parkinson's disease, Huntington's disease, amyotrophic lateral sclerosis, diabetes mellitus, obesity, fatty liver disease, metabolic syndrome, chronic kidney disease, rhytides, elastosis, lentigines, dementia, sarcopenia, and bone marrow failure (BMF) diseases. In some embodiments, the disease, disorder, or condition is selected from Fatty Liver Disease, Obesity, Type 2 diabetes, Alzheimer's disease, Parkinson's disease, Huntington's disease, cardiomyopathy, heart failure, optic neuropathy, mitochondrial myopathy, LEPR deficiency, kidney disease, or Fanconi Anemia. In some embodiments, the disease, disorder or condition is selected from Fatty Liver Disease, Alzheimer's Disease, Dementia, Kidney Disease, or Fanconi Anemia. In some embodiments, kidney disease is chronic kidney disease.

In some embodiments, the disease, disorder or condition is selected from a hematological disease, a metabolic disease. In some embodiments, the disease, disorder or condition is a BMF disease. In some embodiments, the disease, disorder or condition is anemia. In some embodiments, the BMF disease is selected from the group consisting of: Fanconi Anemia, Aplastic Anemia, Diamond-Blackfan Anemia, Dyskeratosis Congenita/Telomere Biology Disorders, GATA2 Deficiency, Myelodysplastic Syndrome, Paroxysmal Nocturnal Hemoglobinuria, Pearson's Disease, SAMD9/SAMD9L Germline Mutations, Severe Congenital Neutropenia, and Shwachman-Diamond Syndrome. In some embodiments, the BMF disease is Fanconi Anemia. In some embodiments, the disease is a BMF disease and the composition comprises a CD44 targeting peptide. In some embodiments, the disease is a BMF disease and the nanoparticle comprises a CD44 targeting peptide.

In some embodiments, the disease, disorder or condition is a metabolic disease. In some embodiments, the metabolic disease is selected from metabolic syndrome, fatty liver disease, obesity, insulin resistance and diabetes. In some embodiments, diabetes is diabetes mellitus. In some embodiments, diabetes is type II diabetes. In some embodiments, the metabolic disease is non-alcoholic fatty liver disease (NAFLD). In some embodiments, the metabolic disease is diabetes. In some embodiments, the metabolic disease is insulin resistance. In some embodiments, the metabolic disease is insulin resistance and diabetes. In some embodiments, the metabolic disease is obesity. In some embodiments, the condition is obesity. In some embodiments, obesity is a rare genetic disease of obesity. In some embodiments, the rare genetic disease of obesity is selected from the group consisting of: Bardet-Biedl syndrome (BBS), Alström syndrome, Proopiomelanocortin (POMC) deficiency, Leptin receptor (LEPR) deficiency, Leptin (LEP) deficiency, Proprotein convertase subtilisin/kexin type 1 (PCSK1) deficiency, Steroid receptor coactivator-1 (SRC1) deficiency, and SH2B adaptor protein 1 (SH2B1) deficiency. In some embodiments, the rare genetic disease of obesity is LEPR. In some embodiments, the rare genetic disease of obesity is LEP.

In some embodiments, the disease, disorder or condition is a neurodegenerative disease. In some embodiments, the neurodegenerative disease is selected from Alzheimer's disease, Parkinson's disease, Huntington's disease, amyotrophic lateral sclerosis (ALS), and dementia. In some embodiments, the neurodegenerative disease is Alzheimer's disease.

In some embodiments, the disease is a cardiovascular disease or condition. In some embodiments, the cardiovascular disease or condition is selected from atherosclerosis, hyperlipidemia, hypercholesterolemia, and hypertriglyceridemia. In some embodiments, the disease is a dermatological condition. In some embodiments, the dermatological condition is selected from rhytides, elastosis, and lentigines. In some embodiments, the disease is a musculoskeletal disease. In some embodiments, the musculoskeletal disease is sarcopenia.

In some embodiments, the disease is a neuromuscular disease. In some embodiments, the neuromuscular disease is selected from Spinal-Bulbar Muscular Atrophy (SBMA), Amyotrophic lateral sclerosis (ALS), Spinal muscular atrophy, Primary lateral sclerosis (PLS), Progressive supranuclea palsy (PSP), Parkinson's disease (PD), Corticobasal degeneration (CBD), Huntington's disease, Charcot-Marie-Tooth disease, Myasthenia Gravis (MG), Congenital myasthenic syndrome, Multiple sclerosis (MS)/Neuromyelitis Optica Spectrum Disorders (NMOSD), Chronic inflammatory demyelinating polyneuropathy (CIDP)/multifocal motor neuropathy (MMN), Inclusion body myositis, Crow-Fukase syndrome, Multiple system atrophy (MSA), Spinocerebellar Degeneration, Moyamoya disease, Subacute sclerosing panencephalitis (SSPE), Progressive multifocal leukoencephalopathy (PML), Fahr disease, Ullrich congenital muscular dystrophy, Distal Myopathy, Bethlem myopathy, Danon disease, Schwartz-Jampel syndrome (SJS), Congenital myopathy, Muscular dystrophy, Periodic paralysis, Syringomyelia, Isaacs syndrome, Hereditary dystonia, Neuroferritinopathy, Cerebral autosomal dominant arteriopathy with subcortical infarcts and leukoencephalopathy(CADASIL), Perry syndrome, Frontotemporal lobar degeneration (FTLD), Acute encephalopathy with biphasic seizures and late reduced diffusion (AESD), Congenital insensitivity to pain with anhidrosis, Alexander disease, Aicardi syndrome, Hemimegalencephaly, Focal cortical dysplasia (FCD), Neuronal migration disorder, Dravet syndrom, Mesial temporal lobe epilepsy with hippocampal sclerosis, Epilepsy with myoclonic absence (EMA), Epilepsy with myoclonic-astatic seizures, Lennox-Gastaut syndrome (LGS), West syndrome, Ohtahara syndrome, Hemiconvulsion-hemiplegia-epilepsy syndrome, Ring chromosome 20 syndrome, Rasmussen encephalitis, PCDH19 epilepsy, Epilepsy with continuous spikes and waves during slow sleep, Landau-Kleffner syndrome, Rett syndrome, Sturge-Weber syndrome, Tuberous sclerosis complex (TSC), Xeroderma pigmentosum, Angelman syndrome, and Glycosylphosphatidylinositol (GPI) anchor deficiency. In some embodiments, the disease is an immune disease. In some embodiments, the immune disease is an autoimmune disease. In some embodiments, the immune disease is selected from: Takayasu arteritis, Giant cell arteritis (GCA), Polyarteritis nodosa (PAN), Microscopic polyangiitis (MPA), Granulomatosis with polyangiitis (GPA), Eosinophilic granulomatosis with polyangiitis (EGPA), Malignant rheumatoid arthritis (MRA), Buerger's disease, Primary antiphospholipid syndrome, APS with venous and, Systemic lupus erythematosus (SLE), Dermatomyositis (DM)/Polymyositis (PM), Mixed Connective-Tissue Disease (MCTD), Sjögren's syndrome, Adult-onset Still's disease (AOSD), Relapsing polychondritis (RP), Behçet's disease, Chronic infantile neurological cutaneous and articular syndrome (CINCA syndrome), TNF receptor-associated periodic syndrome, Familial Mediterranean fever, Nakajo-Nishimura Syndrome, Pyogenic sterile arthritis, pyoderma gangrenosum and acne syndrome, IgG4-related disease, and Eosinophilic sinusitis. In some embodiments, the disease is a hematological disease. In some embodiments, the hematological disease is selected from Aplastic anemia, Autoimmune hemolytic anemia (AHA), Paroxysmal nocturnal hemoglobinuria (PNH), Idiopathic thrombocytopenic purpura, Thrombotic thrombocytopenic purpura (TTP), Primary immunodeficiency syndrome (PIDS), Diamond-Blackfan anemia, and Fanconi anemia. In some embodiments, the disease is an ocular disease. In some embodiments, the ocular disease is selected from Retinitis pigmentosa, Macular degeneration, Leber Hereditary Optic Neuropathy, and Gelatinous drop-like corneal dystrophy. In some embodiments, the disease is selected from Fatty Liver Disease, Alzheimer's Disease, Dementia, Kidney Disease, and Fanconi Anemia. In some embodiments, the disease is selected from Fatty Liver Disease, Obesity, Type 2 diabetes, Alzheimer's disease, Parkinson's disease, Huntington's disease, cardiomyopathy, heart failure, optic neuropathy, mitochondrial myopathy, LEPR deficiency, Kidney disease, and Fanconi Anemia.

In some embodiments, the treating is gene therapy. In some embodiments, the treating comprises providing an exogenous gene to the subject. In some embodiments, the gene is a cDNA. In some embodiments, the gene is devoid of introns. In some embodiments, the gene is a nucleic acid sequence encoding the protein. In some embodiments, the gene is an optimized or variant sequence of the invention. In some embodiments, the gene is a vector comprising the gene. In some embodiments, the increasing comprises administering to the subject a nucleic acid vector encoding the protein. In some embodiments, the vector comprises a cDNA sequence encoding the protein, wherein the cDNA sequence is devoid of introns. In some embodiments, the vector is an expression vector. In some embodiments, the administering is administering a composition of the invention.

The term “expression” as used herein refers to the biosynthesis of a gene product, including the transcription and/or translation of said gene product. Thus, expression of a nucleic acid molecule may refer to transcription of the nucleic acid fragment (e.g., transcription resulting in mRNA or other functional RNA) and/or translation of RNA into a precursor or mature protein (polypeptide).

Expressing a gene within a cell is well known to one skilled in the art. It can be carried out by, among many methods, transfection, viral infection, or direct alteration of the cell's genome. In some embodiments, the gene is in an expression vector such as plasmid or viral vector.

In some embodiments, the increasing comprises delivering the protein to a cytoplasm of the diseased cell. In some embodiments, the increasing comprises administering the protein to the subject. In some embodiments, administering the protein comprises administering a composition of the invention. In some embodiments, the increasing comprises administering to the subject an agonist of the protein. In some embodiments, the increasing comprises delivering into a cytoplasm and/or nucleus of the extracted cells at least one of: a nucleic acid vector encoding the protein, the protein and an agonist of the protein.

In some embodiments, the method comprises receiving cells from the subject. In some embodiments, the cells are obtained from the subject. In some embodiments, the method comprises extracting the cells from the subject. In some embodiments, the cells are disease cells. In some embodiments, the cells comprise mitochondrial stress. In some embodiments, mitochondrial stress is reduced mitochondrial function. In some embodiments, reduced is as compared to a control. In some embodiments, the increasing expression is increasing in the extracted cells. In some embodiments, the extracted cells are contacted with the composition of the invention. In some embodiments, the extracted cells are contacted with a nucleic acid molecule of the invention. In some embodiments, contacted with comprises administering the composition or nucleic acid molecule to the cells. In some embodiments, the extracted cells are returned to the subject. In some embodiments, the returning is after the contacting/administering. In some embodiments, contacting is with the cytoplasm of the extracted cells. In some embodiments, administering is to the cytoplasm of the extracted cells. In some embodiments, the disease cells are target cells. In some embodiments, the extracted cells are target cells.

In some embodiments, the method is an ex vivo method. In some embodiments, the method is an in vivo method. In some embodiments, the method is an in vitro method. In some embodiments, the method comprises receiving disease cells extracted from the subject, increasing expression of the protein in the extracted cells. In some embodiments, the method further comprises returning the extracted cells to the subject.

In some embodiments, the vector comprises a cDNA sequence encoding the protein, wherein the cDNA sequence is devoid of introns. In some embodiments, the vector comprises the sequence of a homolog of the protein. In some embodiments, the vector comprises a sequence encoding a homolog of the protein. In some embodiments, a homolog of the protein is used in place of the protein. In some embodiments, the homolog of the gene is used in place of the gene. In some embodiments, an mRNA of the homolog is used in place of an mRNA of the gene. In some embodiments, a cDNA of the coding sequence (CDS) of a homolog is used in place of a cDNA of the coding sequence (CDS) of the gene.

In some embodiments, a homolog comprises at least 70, 75, 80, 85, 90, 95, 97, 99 or 100% homology to the sequence. Each possibility represents a separate embodiment of the invention. In some embodiments, the sequence is a nucleic acid sequence. In some embodiments, the sequence is an amino acid sequence. In some embodiments, homology is identity. In some embodiments, a homolog comprises at least 70% identity. In some embodiments, a homolog comprises at least 85% identity. In some embodiments, a homolog comprises at least 90% identity. In some embodiments, the homolog retains the function of the gene/protein.

In some embodiments, the method is devoid of a step measuring expression of the protein or a nucleic acid encoding the protein in disease cells of the subject. It will be understood by a skilled artisan that the method of the invention is not merely restoring levels of expression in cells where there was a downregulation. Rather, the gene products administered are beneficial to the subject regardless of their starting levels/basal levels in the disease cells. The gene products are protective and increasing them is beneficial to the subject. This includes increasing them to levels that are higher than basal, e.g., levels that are higher than in healthy (i.e., not diseased) cells. In some embodiments, the subject does not possess diseased cells with decreased expression of the protein as compared to heathy cells of the same tissue or cell type. In some embodiments, the increasing comprises increasing expression of the protein in the diseased cell beyond the expression level in healthy cells of the same tissue or cell type as the diseased cell.

In some embodiments, the treating comprises decreasing lipid accumulation in diseased cells or diseased tissue of the subject. In some embodiments, decreasing is decreasing by at least 10, 15, 20, 25, 30, 35, 40, 45, 50, 55, 60, 65, 70, 75, 80, 85, 90, 95, 97, 99, or 100%. Each possibility represents a separate embodiment of the invention. In some embodiments, the treating comprises increasing the basal metabolic rate in diseased cells or diseased tissue of the subject. In some embodiments, increasing is increasing by at least 10, 20, 30, 40, 50, 60, 70, 80, 90, 100, 200, 300, 400, or 500%. Each possibility represents a separate embodiment of the invention.

In some embodiments, the treating comprises increasing the mitochondrial function of diseased cells or diseased tissue of the subject. In some embodiments, increasing is increasing by at least 10, 15, 20, 25, 30, 35, 40, 45, 50, 55, 60, 65, 70, 75, 80, 85, 90, 95, 97, 99, 100, 200, 300, 400, or 500%. Each possibility represents a separate embodiment of the invention. In some embodiments, the administering is systemic administering. In some embodiments, the administering is local administering to a disease site. In some embodiments, the administering is via a nanoparticle. In some embodiments, the nanoparticle is a lipid nanoparticle.

In some embodiments, the target cell is a disease cell. In some embodiments, a disease cell is a cell of diseased tissue. In some embodiments, the target cell is a disease cell characterized by mitochondrial stress. In some embodiments, the target cell is a disease cell characterized by lipid stress. In some embodiments, the target cell is a disease cell characterized by mitochondrial stress and lipid stress. In some embodiments, the mitochondrial stress and/or lipid stress is characterized by the presence of lipid droplets in the target cell. In some embodiments, the mitochondrial stress and/or lipid stress is characterized by the presence of lipid droplets in a target tissue. In some embodiments, the lipid droplets are intracellular. In some embodiments, the lipid droplets are above the level present in healthy cells or healthy tissue. In some embodiments, the target cell is a disease cell characterized by increased lipids in the disease cell or disease tissue. In some embodiments, the target cell is a disease cell characterized by decreased mitochondrial function. In some embodiments, the target cell is a disease cell characterized by decreased oxygen consumption. In some embodiments, the target cell is a disease cell characterized by a decreased rate of oxygen consumption. In some embodiments, increased is a compared to a control cell. In some embodiments, decreased is as compared to a control cell.

In some embodiments, the target cell is an immune cell. In some embodiments, the target cell is a CD44 positive cell. As used herein, the terms “CD44 positive” and “CD44 expressing” are synonymous and used interchangeably and refer to cells with CD44 protein expressed on their surface. In some embodiments, the target cell is a lymphocyte. In some embodiments, the target cell is neuron. In some embodiments, the target cell is neuronal cell. In some embodiments, the target cell is cardiovascular cell. In some embodiments, the target cell is liver cell. In some embodiments, the target cell is a hepatocyte. In some embodiments, the target cell is a kidney cell. In some embodiments, the target cell is a renal cell. In some embodiments, the target cell is a cardiac cell. In some embodiments, the target cell is a skin cell. In some embodiments, the target cell is a bone cell. In some embodiments, the target cell is bone marrow cell. In some embodiments, the target cell is a hematopoietic cell. In some embodiments, the target cell is a blood cell. In some embodiments, the target cell is an epithelial cell. In some embodiments, the target cell is an endothelial cell. In some embodiments, the target cell is a fibroblast. In some embodiments, the target cell is pancreatic cell. In some embodiments, the target cell is a beta cell. In some embodiments, the target cell is an insulin receiving cell. In some embodiments, the target cell is an adipocyte. In some embodiments, the target cell is a muscle cell. In some embodiments, muscle is skeletal muscle.

In some embodiments, the target cell is selected from liver cells, neural cells, mesenchymal cells, kidney cells, stem cells and bone marrow cells. In some embodiments, the target cell is selected from liver cells, pancreatic cells, neural cells, mesenchymal cells, kidney cells, stem cells and bone marrow cells. In some embodiments, the target cell is a liver cell. In some embodiments, the target cell is a neuronal cell. In some embodiments, the target cell is a mesenchymal cell. In some embodiments, the mesenchymal cell is a muscle cell. In some embodiments, the mesenchymal cell is a fat cell. In some embodiments, a fat cell is an adipocyte. In some embodiments, the target cell is a kidney cell. In some embodiments, the target cell is a renal cell. In some embodiments, the target cell is liver cell. In some embodiments, a liver cell is a hepatocyte.

In some embodiments, the pharmaceutical composition is for use in treating a disease, disorder or condition. In some embodiments, the pharmaceutical composition is for use in treating a disease, disorder or condition characterized by mitochondrial stress in a subject in need thereof. In some embodiments, the disease, disorder or condition is further characterized by lipid stress. In some embodiments, the mitochondrial stress comprises abnormal lipid accumulation in diseased cells or diseased tissue of the subject. In some embodiments, the abnormal lipid accumulation comprises the presence of lipid droplets in the diseased cells or diseased tissue at a level that is increased as compared to healthy cells or tissue.

Provided herein are disease which are known to be characterized by both mitochondrial and lipid stress, these include, but are not limited to: Spinal-Bulbar Muscular Atrophy (SBMA), Amyotrophic lateral sclerosis (ALS), Spinal muscular atrophy, Primary lateral sclerosis (PLS), Progressive supranuclea palsy (PSP), Parkinson's disease (PD), Corticobasal degeneration (CBD), Huntington's disease, Charcot-Marie-Tooth disease, Myasthenia Gravis (MG), Congenital myasthenic syndrome, Multiple sclerosis (MS)/Neuromyelitis Optica Spectrum Disorders (NMOSD), Chronic inflammatory demyelinating polyneuropathy (CIDP)/multifocal motor neuropathy (MMN), Inclusion body myositis, Crow-Fukase syndrome, Multiple system atrophy (MSA), Spinocerebellar Degeneration, Moyamoya disease, Subacute sclerosing panencephalitis (SSPE), Progressive multifocal leukoencephalopathy (PML), Fahr disease, Ullrich congenital muscular dystrophy, Distal Myopathy, Bethlem myopathy, Danon disease, Schwartz-Jampel syndrome (SJS), Congenital myopathy, Muscular dystrophy, Periodic paralysis, Syringomyelia, Isaacs syndrome, Hereditary dystonia, Neuroferritinopathy, Cerebral autosomal dominant arteriopathy with subcortical infarcts and leukoencephalopathy(CADASIL), Perry syndrome, Frontotemporal lobar degeneration (FTLD), Acute encephalopathy with biphasic seizures and late reduced diffusion (AESD), Congenital insensitivity to pain with anhidrosis, Alexander disease, Aicardi syndrome, Hemimegalencephaly, Focal cortical dysplasia (FCD), Neuronal migration disorder, Dravet syndrom, Mesial temporal lobe epilepsy with hippocampal sclerosis, Epilepsy with myoclonic absence (EMA), Epilepsy with myoclonic-astatic seizures, Lennox-Gastaut syndrome (LGS), West syndrome, Ohtahara syndrome, Hemiconvulsion-hemiplegia-epilepsy syndrome, Ring chromosome 20 syndrome, Rasmussen encephalitis, PCDH19 epilepsy, Epilepsy with continuous spikes and waves during slow sleep, Landau-Kleffner syndrome, Rett syndrome, Sturge-Weber syndrome, Tuberous sclerosis complex (TSC), Xeroderma pigmentosum, Angelman syndrome, Glycosylphosphatidylinositol (GPI) anchor deficiency, Takayasu arteritis, Giant cell arteritis (GCA), Polyarteritis nodosa (PAN), Microscopic polyangiitis (MPA), Granulomatosis with polyangiitis (GPA), Eosinophilic granulomatosis with polyangiitis (EGPA), Malignant rheumatoid arthritis (MRA), Buerger's disease, Primary antiphospholipid syndrome, APS with venous and, Systemic lupus erythematosus (SLE), Dermatomyositis (DM)/Polymyositis (PM), Mixed Connective-Tissue Disease (MCTD), Sjögren's syndrome, Adult-onset Still's disease (AOSD), Relapsing polychondritis (RP), Behcet's disease, Chronic infantile neurological cutaneous and articular syndrome (CINCA syndrome), TNF receptor-associated periodic syndrome, Familial Mediterranean fever, Nakajo-Nishimura Syndrome, Pyogenic sterile arthritis, pyoderma gangrenosum and acne syndrome, IgG4-related disease, Eosinophilic sinusitis, Aplastic anemia, Autoimmune hemolytic anemia (AHA), Paroxysmal nocturnal hemoglobinuria (PNH), Idiopathic thrombocytopenic purpura, Thrombotic thrombocytopenic purpura (TTP), Primary immunodeficiency syndrome (PIDS), Diamond-Blackfan anemia, Fanconi anemia, Retinitis pigmentosa, Macular degeneration, Leber Hereditary Optic Neuropathy, and Gelatinous drop-like corneal dystrophy. In some embodiments, the disease, disorder or condition is selected from Fatty Liver Disease, Alzheimer's Disease, Dementia, Kidney Disease, or Fanconi Anemia. In some embodiments, the disease is Fanconi Anemia.

In some embodiments, the subject does not possess diseased cells with decreased expression of the protein as compared to heathy cells of the same tissue or cell type. In some embodiments, the treating comprises increasing expression of the protein in a diseased cell of the subject beyond the expression level in healthy cells of the same tissue or cell type as the diseased cell. In some embodiments, the treating comprises decreasing lipid accumulation in diseased cells or tissue of the subject.

### Other Methods

According to another aspect, there is provided a method of identifying a gene for use in gene therapy to treat a disease, disorder or condition characterized by mitochondrial stress, the method comprising:
a. receiving a population of cells;
b. decreasing expression of a plurality of genes in the population of cells wherein each cell has decreased expression of only one gene of the plurality to produce a population of knockdown cells;
c. placing the population of knockdown cells in a condition of metabolic stress;
d. selecting a cell of the population of knockdown cells with a negative phenotype; and
e. identifying the gene of the plurality of genes with decreased expression in the selected cell;
thereby identifying a gene for use in gene therapy.

In some embodiments, the method is an in vitro method. In some embodiments, the method is an ex vivo method. In some embodiments, the population of cell is of the same cell type as is affected by the disease, disorder or condition. In some embodiments, the decreasing comprises a genome-wide molecular screen performed in the received population of cells. In some embodiments, the genome-wide screen is a genome-wide CRISPR knockout screen.

In some embodiments, the cells are human cells. In some embodiments, the cells are healthy cells. In some embodiments, the cells are not disease cells. In some embodiments, the cells are not cell line cells. In some embodiments, the cells are primary cells. In some embodiments, the cell are energetic cells. In some embodiments, energetic cells have active mitochondria. In some embodiments, active is active at levels equivalent to healthy cells. In some embodiments, healthy cells are primary cells. In some embodiments, energetic cells are defined by an oxygen consumption rate (OCR) of greater than 50 pmol/min/10≡cells, an OCR (pmol/min)/extracellular acidification rate (ECAR) (mpH/min) greater than 1 or both. In some embodiments, the energetic cells are E6/E7LOW Hepatocytes.

In some embodiments, the negative phenotype is decreased proliferation. In some embodiments, the negative phenotype is cell death. In some embodiments, the negative phenotype is increased intracellular lipid accumulation in the population of knockdown cells. In some embodiments, the increased intracellular lipid accumulation comprises the presence of lipid droplets in the population of knockdown cells at a level that is increased as compared to non-knocked-down cells. In some embodiments, the negative phenotype is decreased mitochondrial function. In some embodiments, the negative phenotype is decreased oxygen consumption. In some embodiments, the negative phenotype is decreased a decreased rate of oxygen consumption. In some embodiments, the negative phenotype is selected from increased mitochondrial superoxide, differential mitochondrial membrane potential, increased mitochondrial HSP60/70 expression, increased protein carbonyl content, increased lipid peroxidation, and increased thiobarbituric acid reactive substances. In some embodiments, increased is as compared to non-knocked-down cells. In some embodiments, decreased is as compared to non-knocked-down cells. Methods of measuring these phenotypes are well known in art and kits/assays for performing these measurements are commercially available. Any method of determining the negative phenotype such as is known in the art is envisioned.

According to another aspect, there is provided a method of targeting an agent to a CD44 positive cell, the method comprising producing a composition comprising the agent and a peptide of the invention and contacting the CD44 positive cell with the composition, thereby targeting an agent to a CD44 positive cell.

In some embodiments, the composition comprises a fusion protein of the invention. In some embodiments, the composition comprises a nanoparticle and the peptide is on or attached to the surface of the nanoparticle. In some embodiments, the agent is within the nanoparticle. In some embodiments, the agent is within the core of the nanoparticle. In some embodiments, the agent is within the shell of the nanoparticle. In some embodiments, the agent is conjugated to the nanoparticle. In some embodiments, the peptide is conjugated to the nanoparticle. In some embodiments, conjugated is conjugated to an outer surface of the nanoparticle. In some embodiments, the composition is a pharmaceutical composition.

In some embodiments, the agent is a drug. In some embodiments, the agent is a nucleic acid molecule. In some embodiments, the agent is a vector. In some embodiments, the agent is a gene therapy. In some embodiments, the agent is a therapeutic molecule. In some embodiments, the agent is a small molecule. In some embodiments, the agent is active in the cytoplasm of the CD44 positive cell. In some embodiments, the agent is active in the nucleus of the CD44 positive cell. In some embodiments, the agent is active in the interior of the CD44 positive cell. In some embodiments, a CD44 positive cell is a CD44 expressing cell.

In some embodiments, the method is an in vitro method. In some embodiments, the method is an ex vivo method. In some embodiments, the CD44 cell is in a subject. In some embodiments, the method is an in vivo method. In some embodiments, the method is a therapeutic method. In some embodiments, the contacting comprises administering the composition to the subject.

By another aspect, there is provided a method of producing a nanoparticle, the method comprising: providing a nanoparticle comprising a peptide of the invention and loading the nanoparticle with an agent, thereby producing a therapeutic nanoparticle.

In some embodiments, the nanoparticle comprises a fusion protein of the invention. In some embodiments, the nanoparticle targets to CD44 expressing cells. In some embodiments, the nanoparticle is a therapeutic nanoparticle. In some embodiments, the agent is a therapeutic agent. In some embodiments, the agent is a drug. In some embodiments, the agent is a therapeutic molecule. In some embodiments, the agent is a gene therapy agent. In some embodiments, the agent is a nucleic acid molecule. In some embodiments, the agent is a vector. In some embodiments, the peptide is on or attached to a surface of the nanoparticle. In some embodiments, a surface is an outer surface. In some embodiments, the agent is within the nanoparticle. In some embodiments, the agent is within the core of the nanoparticle. In some embodiments, the agent is within the shell of the nanoparticle. In some embodiments, the agent is conjugated to the nanoparticle. In some embodiments, the peptide is conjugated to the nanoparticle. In some embodiments, conjugated is conjugated to a surface of the nanoparticle.

As used herein, the term “about” when combined with a value refers to plus and minus 10% of the reference value. For example, a length of about 1000 nanometers (nm) refers to a length of 1000 nm+-100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, or claims, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

As used in this specification and the appended claims, the singular forms “a,” “an,” and “the” include plural referents, unless the context clearly dictates otherwise. The terms “a” (or “an”) as well as the terms “one or more” and “at least one” can be used interchangeably.

Furthermore, “and/or” is to be taken as specific disclosure of each of the two specified features or components with or without the other. Thus, the term “and/or” as used in a phrase such as “A and/or B” is intended to include A and B, A or B, A (alone), and B (alone). Likewise, the term “and/or” as used in a phrase such as “A, B, and/or C” is intended to include A, B, and C; A, B, or C; A or B; A or C; B or C; A and B; A and C; B and C; A (alone); B (alone); and C (alone).

Wherever embodiments are described with the language “comprising,” otherwise analogous embodiments described in terms of “consisting of” and/or “consisting essentially of” are included.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### Examples

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization—A Laboratory Course Manual” CSHL Press (1996); all of which are incorporated by reference. Other general references are provided throughout this document.

### Materials and Methods

Cell culture: The E6/E7LOW human hepatocytes were maintained, expanded and differentiated on collagen type I (354236, Corning) tissue culture grade vessels. In brief, differentiated cells were cultured in medium comprised of William's E medium (W4125, Sigma) supplemented with 3.75 gr/L BSA, fraction V (02160069-CF, MP biomedicals), dexamethasone (D2915, Sigma-Aldrich, USA), Insulin-Transferrin-Selenium (ITS-G, 41400045, Gibco, USA) and penicillin-streptomycin (15140122, Gibco, USA). Proliferating cells were cultured in the above medium, further supplemented with 10 ng/ml Oncostatin M (OSM, 300-10H-100, PeproTech) designated as proliferation medium. Medium changes while in proliferation are every day and when in differentiation/maintenance, every other day. Proliferating cells are passaged at about 80% confluency. Differentiation of cells is induced by culture in medium devoid of OSM at about 80% confluency. The cells are used after at least 4 days in differentiation medium.

HEK 293T cells were cultured in DMEM, high glucose (D5671, Sigma), supplemented with 10% FBS (04-127-1A, Biological Industries), L-Alanyl-L-Glutamine (Stable Glutamine) (03-022-1B, Biological industries) and penicillin-streptomycin (15140122, Gibco, USA).

All cells were cultured under standard conditions at 37° C. and 5% CO2 in a humidified incubator.

Immunofluorescence, lipid staining: The cells were fixed in 4% paraformaldehyde (PFA) for 15 min at RT. After washing away the PFA the fixed cells are permeabilized with 0.2% Triton-X 100 for 30 min and then blocked with 1% BSA (fraction V) in PBS-Tween for 1h at RT. Blocking is accompanied by glycine 300 M treatment to quench unreacted PFA. The primary antibody is added at the required dilution in blocking solution O/N at 4° C. The primary antibody is removed, and the cells are washed with PBS-T (3×5 min each). Secondary antibody is added at 1:200 routinely in PBS for 2 h at RT. The cells are ready for microscopy after extensive washing of the second antibody (≥5×7 min RT).

Lipids were detected by staining with the lipophilic vital dye Nile-red (72485, Sigma). The dye was used at 2 μg/ml final concentration with 2 μg/ml Hoechst 33342 (B2261, Sigma) counter stain for 30 min. When used for staining live cells, both dyes were added directly to the culture medium and incubated at 37° C. for 30 min.

Imaging was performed on a Zeiss Axiovert inverted fluorescent microscope, equipped with the LSM700 confocal array. For lipid content mass assessment, images were taken on an Olympus IX81 inverted fluorescence microscope with scripted acquisition of 3×3 tiled images centered at the center of each well in 96 well format. The resulting images were exported in the microscope control software (CellSens) as raw .tiff files for fluorescent intensity measurement and nuclei count. The 3×3 images per well were finally stitched together with the CellSens multiple image alignment tool to create display images per well, providing a representative depiction of each well.

FIJI was used for scripted mass background reduction and fluorescent intensity measurement. CellProfiler was used for nuclei count based on RAW images of the Hoechst channel. The primary object count tool was used with a cut-off of 15-60 pixels.

E6/E7LOW human hepatocytes puromycin toxicity curves: Puromycin (13884, Cayman chemical) was two-fold serially diluted from 10 μg/ml to 0.3 μg/ml in proliferation medium and applied to E6/E7LOW human hepatocytes growing in 96-well plate format. The cells were monitored and imaged daily and on the 3rd day post exposure were analyzed with a LIVE/DEAD assay (L3224, ThermoFischer). The puromycin concentration killing practically all cells in ˜3 days was defined as 2.5 μg/ml for the 653 (female) genotype and 5 g/ml for the 422 (male) genotype cells.

RNAseg and RT-gPCR: RNA was extracted using the NucleoSpin RNA II kit (Macherey-Nagel) or the QuickRNA miniprep kit (R1054, Zymo research) according to the manufacturer's instructions. The extracted RNA was quantified on a Nanodrop 1000 and 1 g was reverse transcribed to cDNA with the qScript cDNA SuperMix (Quanta BioSciences) for use in RT-qPCR or used for library construction and RNAseq (Genomic Technologies Facility, HUJI). RT-qPCR was performed using KAPA SYBR FAST qPCR Master Mix Kit (KAPA biosystems) on a QuantStudio 5 Real-Time PCR System (Applied Bioscience, Cat number: AB-A28574). RNAseq was performed on an Illumina NextSeq 500 platform with read length of 75 and Novasec with read length of 50 nt.

RT-qPCR results were analyzed through ΔΔCt against the RPL32 housekeeping gene and plotted.

RNAseq reads were mapped to human reference genome GRCh38.p13 with v33 annotations from Gencode, using the RNA STAR tool version 2.7.2b of the Galaxy-Europe platform. Dataset GSE143260 was processed through the same pipeline to generate raw counts for 2D HepaRG cells. The remaining raw counts used are from (Ardisasmita et al., “A comprehensive transcriptomic comparison of hepatocyte model systems improves selection of models for experimental use”, Commun. Biol., 2022 Oct. 14; 5(1):1094, the contents of which are hereby incorporated by reference in their entirety). All raw counts were compiled in a single table and normalized through ExpressAnalyst, using the log 2 per million option. Differential analysis was performed in ExpressAnalyst using the DESeq2 option.

Heatmaps of gene expression levels were created with the Morpheus tool (Broad Institute). PCA analysis was performed on ExpressAnalyst. Pathway enrichment analysis was performed on Metascape with default options and pathway visualizations in STRING.

CRISPR plasmid library amplification: The GeCKO v2 Human CRISPR Knockout Pooled Library (#1000000048, Addgene) was used. The two half-libraries, A and B, were amplified separately by electroporation in Endura electrocompetent cells (60242-1, Lucigen/LGC). In brief, the cells were thawed on ice and 1 μl from each 50 ng/μl half-library DNA stock were mixed with 25 μl cells. Each mix was placed in a pre-cooled 1 mm cuvette and electroporated on a Gene Pulser II, equipped with Pulse Controller II (Bio-Rad) at 600 Ohm resistance, 10 μF capacitance and 1800 V pulse. The cells were quickly recovered in 975 μL warm recovery medium. A total of 4 electroporations were performed per half-library according to the manufacturer's instructions. The recovered cells were pooled and incubated at 37° C. for ˜1.5 h with vigorous shaking (250 rpm) and afterwards were plated in ampicillin (100 μg/ml) LB-agar plates: 200 μL transformed cells per plate. The plates were incubated at 30° C. overnight (˜16h).

The colonies were harvested in 750 μl LB-medium per plate using cell spreaders. The bacteria were pooled per half-library, centrifuged at 5000 rpm for 5 min at RT and the pellets were weighted. Based on the weight, a suitable number of maxi-prep columns was used per half-library. Plasmid/library DNA was isolated using the ZymoPURE II Plasmid Maxiprep Kit (D4203, Zymo research), according to manufacturer's instructions.

CRISPR lentivirus library production: HEK 293T cells were grown to ˜90% confluency in 8×15 cm plates (639160, Greiner). A mix 1:1 w/w of the half-libraries A and B was prepared, and each plate was transfected with 15.96 μg CRISPR library A+B mix, 7.92 μg pMD2.G (#12259, addgene) and 12 μg psPAX2 (#12260, addgene) complexed with 110 μL Trans-IT LT1 reagent (MIR-2300, MirusBio) in 2 ml OptiMEM (31985-047, Gibco). The mix was incubated at RT for 25 min and then added dropwise to the 293T flasks. The viral supernatant was harvested at 71 h post transfection and centrifuged at 900×g for 5 min at RT to pellet cell debris. The clarified supernatant—CRISPR lentivirus library was further filtered through a 0.45 μm PVDF membrane (syringe filters, SLHV033RS, Millipore/Merck), aliquoted and stored at −80° C.

Viral stocks titration: Cells were seeded on a 96-well plate and infected at 70-80% confluency. The viral stock was diluted two-fold, starting from 40% of the total medium volume used. Routinely 7 dilutions are used, representing the ratios 40-0.31%. Uninfected wells serve as positive and negative control. Polybrene (Merck-Sigma) was found to significantly enhance transduction efficiency at a concentration of 8 μg/ml in both 653 and 422 genotypes and was routinely used. The cells are incubated with the virus for ˜24h after which the medium is replaced with standard growth medium. Following an extra overnight (O/N) incubation the medium is changed to puromycin-containing medium to all wells except the uninfected positive control and the cells are imaged daily. On the 2nd or 3rd day a LIVE/DEAD assay is performed to quantify the relative cell death, compared to the uninfected cells. The % cell viability per viral stock dilution provides an estimate of the Multiplicity of Infection (MOI).

CRISPR cell library construction: In order to ensure that infected cells will be transduced by a single viral particle only, a MOI of 0.3 was used. For ˜500× over-representation of cells to sgRNA, passage 7 E6/E7LOW human hepatocytes were gradually upscaled to 205×10\hat{ }6 cells and infected at the above MOI (1:100 dilution of the CRISPR lentivirus library stock stock). The cells were cultured to ˜80% confluency with continuous puromycin selection and then made into single-cell suspension with trypsin/EDTA processing and mixed thoroughly. Post mixing, the cells were seeded at 80+% confluency and were induced to differentiation for 4 days, again with continuous antibiotic selection. The cells were cultured for more than 7 days after transduction with the library, to ensure efficient knock-out of the target genes. Frozen stocks of the library were also kept.

Preparation of fatty acid stocks and steatosis induction: Sodium oleate and sodium palmitate were prepared from Oleic (W281506, Sigma) and Palmitic (P0500, Sigma) acid. In brief, oleic or palmitic acid were mixed with 1 N NaOH to a final concentration of 0.1 M of fatty acid in 0.1 N NaOH. The oleic acid solution was heated to 37° C. and palmitic to 86° C. to assist dissolution. After 2-3 hours clear solutions of sodium oleate or palmitate were recovered. The solutions were aliquoted and stored at −80° C. Oleate 0.1 M solution is warmed to RT and palmitate to ˜80° C. to liquify prior to use.

The hepatocytes were exposed to metabolic stress caused by Western-style diet emulated with high-fat and high-glucose medium (HFG condition) or a diabetes-like condition emulated with high glucose medium (HG condition). For HFG, a specialized version of differentiation medium was used, containing 4% fatty-acid low BSA, instead of the standard described above. A 1:1 molar ratio mix of oleate and palmitate (fatty acids, FA) was mixed with the above medium at a 7:1 FA:BSA molar ratio. A 48-hour incubation with the high-fat medium was used. For the HG condition the cells were maintained in culture for 2 (653 cells) or 3 (422 cells) weeks.

In addition to the HFG steatosis described, another mode was also developed. While assessing the long-term maintenance of differentiated hepatocytes, there was noticed a significant increase in lipid content at ˜2 weeks for the 653 genotype and ˜3 weeks for the 422. The standard culture medium contains hyperglycemic amounts of glucose and high concentration of insulin and these conditions have been causally associated with increased lipid accumulation and dysregulated insulin response in primary hepatocytes.

Experimental set-up and cell sorting: The GeCKO v.2 cell library was divided into 3 experimental populations: control, HFG and HG cells. 3 biological replicates were used for control and HFG and 2 for HG for each cell type (female-653 and male-422). After steatosis induction, as described above, the cells were stained in their cell containers, processed into a single-cell suspension and sorted. Fixation of the cells in suspension prior to cell sorting led to significant losses in test experiments due to problematic sedimentation of the cells during the wash steps. To avoid such loses the cells were stained and sorted live. The cell mix was routinely passed through a 100 m mesh to remove cell aggregates prior to sorting.

The cells were sorted using a FACS Aria III (BD biosciences). Nile red staining produced a dose-dependent fluorescence intensity emission in the green channel (488 nm). Sorting gates were set on this channel, to isolate the highest fluorescing 18% of cells and lowest fluorescing 25%. This set-up provided ˜2-3×10\hat{ }6 events respectively (high-/low-lipid) per GeCKO v.2 library cell container. A total of 7 containers were used for each experimental condition to ensure proper sgRNA representation. The sorted cells were pooled per condition and centrifuged at 8000 rpm for 15 min at 4° C. The pellets were stored at −80° C.

gDNA isolation & DeepSeq preparation: Hepatocyte genomic DNA (gDNA) was isolated as previously described (Chen et al., “Genome-wide CRISPR screen in a mouse model of tumor growth and metastasis”, PMID: 25748654, the contents of which are herein incorporated by reference in their entirety). In brief, the cell pellets were thawed on ice and resuspended in 3 ml NK lysis buffer (50 mM Tris, 50 mM EDTA, 1% SDS, pH 8). 15 μL from a 20 mg/ml Proteinase K stock were added the next day and the lysate was incubated at 55° C. overnight. 7.5 μL RNase A (T3018L, NEB) were added to the lysate, mixed and incubated at 37° C. for 30 min. Samples were cooled on ice and 2 ml of pre-chilled 7.5M ammonium acetate (Sigma A1542) were added to precipitate the proteins. The samples were vortexed for ˜20 sec at high speed and then centrifuged at ≥4,000×g for 10 min. 3 ml 100% isopropanol were added to the tube to wash the pellet, mixed and centrifuged at ≥4,000×g for 10 min. 3 ml of 70% ethanol were added, mixed and then centrifuged at ≥4,000×g for 1 min. The pellet was air-dried for 10-30 min and the gDNA was resuspended in 250 μl of molecular grade water. The tube was incubated at 65° C. for 1 hr and also at room temperature with periodic agitation to completely resuspend the DNA. The high-lipid samples were processed with 6 ml of NK buffer and all volumes scaled accordingly. gDNA concentrations were measured on a Nanodrop 1000 (Thermo Scientific).

The sgRNA insertions in the hepatocyte genomic DNA were amplified in two consecutive PCR reactions. The first—PCR 1—amplified the sgRNA areas and attached adaptors, utilized by the second—PCR 2—which provided the sequences necessary for attachment to the flow cell, barcoding etc.

Multiple PCR 1 reactions per condition were carried out using the high fidelity Herculase II Fusion DNA Polymerase (600679, Agilent) with 4 g gDNA template each to achieve a high sgRNA representation. All reactions per condition were pooled together and 50 μl were run on a 2% agarose gel. The dominant ˜200 bp band was excised and the gel extracted using QIAquick Gel Extraction Kit (28704, Qiagen), according to the manufacturer's instructions. PCR 2 used 9 ng of purified PCR 1 product as template and generated the Nextera DNA library to be sequenced. PCR reaction composition is provided below.

Using a preheated lid, the amplification is done in the following cycle:
72° C. for 3 minutes
95° C. for 30 seconds
12 cycles of:
95° C. for 10 seconds
55° C. for 30 seconds
72° C. for 30 seconds
72° C. for 5 minutes
Hold at 10° C.

PCR 1

| | | |
|---|---|---|
| 5x buffer | 10 | μl |
| dNTPs (25 mM) | 0.5 | μl |
| Forward DeepSeq primer | 2 | μl |
| Reverse DeepSeq primer | 2 | μl |
| Herculase II | 0.5 | μl |
| gDNA template | 4 | μg |

| | |
|---|---|
| ddH2O | to 50 μl |

PCR 2

| | | |
|---|---|---|
| 5x buffer | 6 | μl |
| dNTPs (25 mM) | 0.3 | μl |
| Forward Nextera primer | 3 | μl |
| Reverse Nextera primer | 3 | μl |
| Herculase II | 0.3 | μl |
| PCR1 template | 9 | ng |

| | |
|---|---|
| ddH2O | to 30 μl |

The amplified sgRNA Nextera libraries from three biological replicate experiments were sequenced using NextSeq 500/550 High Output Kit v2.5 (75 Cycles) (20024906, Illumina).

Analysis of sequenced data: All raw sequence files per sample were concatenated to a single file. Human library A and Human library B sgRNA sequence files, containing the sequences of all sgRNAs in the GeCKO v.2 human library, were downloaded from Addgene (ver. 9mar.2015, Addgene) and concatenated to create the reference library. The columns were rearranged from left to right as sgRNA ID, sgRNA sequence and target gene ID, to be compatible with the MAGeCK pipeline. The raw sequence files and the reference library were used with the MAGeCK count tool to provide count tables. A master count table was produced containing all samples.

The resulting count table was used with the MAGeCK-MLE pipeline which applies the Maximum Likelihood Estimation approach to assess the essentiality of genes in multiple conditions. All high-lipid samples were evaluated together—the same for low-lipid samples. All experimental conditions were compared to the respective controls.

The relative sgRNA enrichment or depletion represents enrichment or depletion of gene knockouts. To create a working ranking, only genes with FDR-adjusted p-value ≤0.01 in at least one condition were selected. The genes were processed separately for the two experimental set-ups i.e. HFG and HG. Given the assay outputs results for the same genes in multiple conditions, a single set of beta and p.values encompassing the overall gene knockout ranking would be essential for proper assessment. In order to combine all the metrics for any given gene, beta scores in the various eligible conditions were combined as a sum of absolute values, designated as cumulative beta-score. p.values <0.05 were combined using the Fisher method in a cumulative p.value.

The aforementioned list was subjected to pathway enrichment analysis using Metascape at standard settings. Genes from the highest-ranking pathways as well as pathways connected with liver steatosis were picked for individual validation. All selected genes were the highest ranking based on the MLE pipeline and/or significant partners of said genes.

Individual sgRNA cloning & arrayed CRISPR: Arrayed CRISPR is used for the validation of the phenotypic effects for selected high-ranking genes. The process starts with cloning the targeting sgRNA in the proper vector for lentivirus production. Two sgRNAs per gene are selected from the GeCKO v2 library and are synthetized (Syntezza, IDT) and cloned into the lentiCRISPR v2 vector (#52961, Addgene) according to the GeCKO library protocols. In brief the oligos are annealed, phosphorylated and ligated to a BsmBI-digested plasmid. The ligated plasmids are transformed in Stbl3 cells prepared with the Mix n Go kit (T3001, Zymo research), plated in agar-LB plates with 100 μg/ml ampicillin and incubated overnight at 37° C. Next day a single colony is grown in suspension in 6 ml LB with antibiotic selection. Plasmid DNA is extracted (ZymoPURE Plasmid Miniprep, D4209, Zymo research) and Sanger sequenced with primer LKO.1 (Genomic Technologies Facility, The Alexander Silberman Institute of Life Science, HUJI) to verify the correct placement of the sgRNA in the vector.

The individual-gene knockout lentivirus stocks are produced as described for the CRISPR lentivirus library (scaled to the volumes required).

E6/E7LOW human hepatocytes are infected at MOI 1 and handled as the CRISPR cell library cells. When differentiated they are processed according to the HFG or HG condition and then assessed through epifluorescence. Cell nuclei numbers are estimated by counting of Hoechst-positive objects in CellProfiler using a cut-off of 15-60 pixels for primary object size. Relative Nile red stained, lipid fluorescence intensity at 488 nm is normalized to the nuclei numbers calculated and compared to the non-targeting (NT/Control) sgRNA lentivirus transduction. The samples are assessed as triplicates or quadruplicates.

Western: The cells were lysed with RIPA buffer and the total protein was evaluated using BCA kit. 25 ug of total protein was then run on a 4-12% Bis-Tris gels. The proteins were transferred to a nitrocellulose membrane and then blotted using specific antibodies.

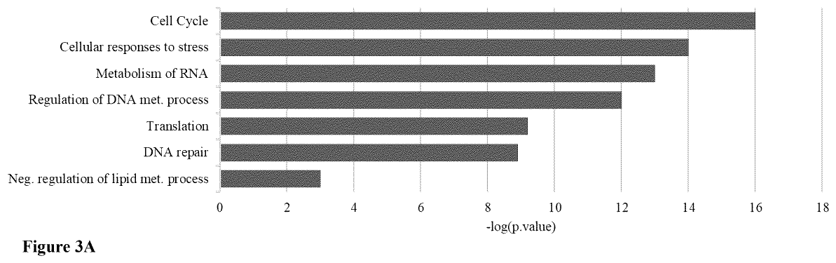

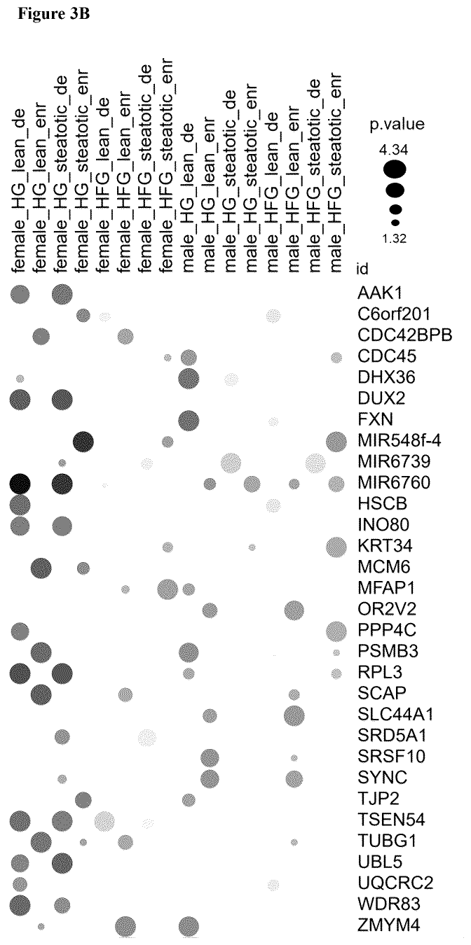

Pathway enrichment analysis: By using the gene knockout results of HFG or HG with cumulative p.value <0.001 to probe the other assays'<0.01 genes 1440 high-raking common genes are identified (FIG. 3A).

Histology protocol: Hematoxylin and eosin staining was performed on 4 μm formalin fixed and paraffin embedded sections using the Leica ST5010 auto-stainer (Leica Biosystems Newcastle Ltd, UK). Slides were dewaxed, rehydrated and incubated for 10 minutes in Hematoxylin followed by incubation in Eosin for 2 minutes. Slides were dehydrated, cleared in Xylene and mounted with cover slips. Masson Trichrome (MTC), Congo Red (CR) and Cresyl Violet (CEA500) stains were performed using commercial kits (all from Scytek USA) according to the manufacturer's instructions.

Blood counts and biochemistry: Blood counts, bone marrow counts and biochemistry analysis were performed by the Hadassa diagnostic lab according to standard protocols.

Quantification of metabolic function: Mitochondrial function was measured using the Seahorse XF Cell Mito stress test kit according to manufacturer instructions (Agilent). Briefly, cardiac organoids or cells were seeded on Seahorse XFp mini plates coated with 1% Matrigel. Cells were allowed to acclimate for 24 h. Cultures were then incubated in unbuffered XF base medium supplemented with 2 mM glutamine, 1 mM sodium pyruvate and 10 mM glucose (pH 7.4) for 1 h at 37° C. in a non-CO2 incubator. The basal oxygen consumption rate (OCR) was measured for 30 min, followed by injection of 1 M oligomycin, a mitochondrial complex V inhibitor that blocks oxidative phosphorylation. The decrease in OCR due to oligomycin treatment was defined as the oxidative phosphorylation rate. Carbonyl cyanide 4-(trifluoromethoxy) phenyl hydrazone (0.5 M), an uncoupling agent, was added at 60 min to measure maximal mitochondrial activity, and complete inhibition was induced at 90 min using a mixture of 0.5 M antimycin A and rotenone, mitochondrial complex III and mitochondrial complex I inhibitors. The results were normalized to the total cell number per well. The cells remained at approximately the seeding numbers by the time of assay as determined by Hoechst staining after the Mito stress assay. In brief, two separate fields per well were imaged on an Olympus X81 microscope, the raw images were exported, and cell nuclei were counted on Cell Profiler as primary objects.

For cell counting, genomic (g)DNA was extracted from cells using the Quick-DNA Miniprep Plus kit (D3024, Zymo) according to manufacturer instructions. gDNA concentration and total gDNA extracted per sample were measured using a Nanodrop 1000 spectrophotometer. To find the number of cells, qPCR was utilized with human-specific primers, amplifying a 156 bp region of gene EDEM1. The templates from cells were 2-fold diluted from 10 ng to 0.625 ng per reaction and all samples/dilutions were assessed in quadruplicates. The average cycle threshold (Ct) values per template quantity were plotted and samples with comparable slopes/efficiencies were used for the analysis.

Viral infection: On the day of infection, 5.5 ul polybrene is added into 5.5 ml of each of the UBL5 and Control viral stocks, and 1 ul polybrene is added into 1 ml of the lenti-GFP stock. Medium is discarded from cells in a 96-well plate and 50 ul of each viral stock is added into the appropriate well. 400 ul of medium is added after. After a 20-minute incubation with the virus at 37° C., the plate is centrifuged for 30 minutes at 800 g at 32° C. The medium is removed, and fresh medium is added (optionally with Mitomycin C). Fresh medium is added every 48 hours.

Comet assay: The comet assay was performed according to the standard protocol as described in Clementi et al., 2021, “Measuring DNA damage using the alkaline comet assay in cultured cells”, Bio-protocol, 11(16): e4119, the contents of which are hereby incorporated by reference herein in their entirety.

Organoid generation: Upcyte hepatocytes 653 at passage 8 and microvascular endothelial cells were mixed at 1:1 ratio and centrifuged at 300×g for 5 min. Cell pellet was mixed with ruthenium-phenanthroline-based phosphorescence dye 50 μm diameter microbeads (CPOx-50-RuP) microbeads (Colibri Photonics, Germany) for subsequent interstitial oxygen measurements and re-suspended in 100 μl of ice-cold Collagen Type I (Corning, CA). PDMS microwells were loaded with 1.35 μl of Collagen suspension containing 5×10⁴ cells and oxygen-sensing beads and incubated for 30 min at 37° C. for polymerization. E6/E7^{LOW} human hepatocyte culture medium supplemented with 5 ng/ml vascular endothelial growth factor (VEGF) (Peprotech, USA) was added for overnight incubation at 37° C., allowing the cells to self-assemble into organoids 600±100 μm in diameter.

Inflammatory cytokines secretion—Antibody array: Inflammatory cytokines secreted by hepatic organoids in culture media supernatants was quantified at week 10 using the Human Inflammation Array Q3 from RayBiotech (Cat. #QAH-INF-3) according to the manufacturer's protocol. Hepatic organoids culture media that was not in contact with the tissues was used as negative control. The slide was sent to the manufacturer for scanning and measurement of Cy3 fluorescence. Each measured molecule has a standard curve from which their concentrations were calculated in the samples.

Transmission electron microscopy: Hepatic organoids were fixed in 2% paraformaldehyde with 2.5% glutaraldehyde in 0.1M Cacodylate buffer (pH 7.4) for 5 hours at room temperature and moved to 40 C to continue fixing overnight. The organoids were then washed 4 times, 10 minutes for each wash, in cacodylate buffer. Organoids were then post fixed and stained with 1% osmium tetroxide, 1.5% potassium ferricyanide in 0.1M cacodylate buffer for 1 hour. Organoids were then washed 4 times in cacodylate buffer followed by dehydration in increasing concentrations of ethanol consisting of 30%, 50%, 70%, 80%, 90%, 95%, 10 minutes for each step followed by 100% anhydrous ethanol 3 times, 20 minutes each, and finally propylene oxide 2 times, 10 minutes each. Following dehydration, the organoids were infiltrated with increasing concentrations of Agar 100 resin in propylene oxide, consisting of 25, 50, 75, and 100% resin for 16 hours each step. The organoids were then embedded in fresh resin and placed in an oven at 600 C for 48 hours for polymerization.

Embedded organoids in blocks were sectioned with a diamond knife on a Leica Reichert Ultracut S microtome. Ultrathin sections (80 nm) were collected onto 200 Mesh, thin bar copper grids. The sections on grids were sequentially stained with uranyl acetate for 10 minutes and Lead citrate for 10 minutes. Sections were imaged with the electronic microscope Tecnai 12 TEM 100 kV (Phillips) equipped with MegaView II CCD camera and Analysis® version 3.0 software (SoftImaging System GmbH).

Lipid accumulation assay: Quantification of lipid accumulation was performed using the HCS LipidTOX Phospholipidosis and Steatosis Detection Kit (Thermo Fisher Scientific, USA). Cells were fixed in 4% PFA and stained with 1× neutral lipid detected reagent for 45 min and counterstained with Hoechst 33258 (1 μg/ml). Staining intensity was normalized to the number of Hoechst 33258-positive nuclei.

### Example 1: Screen for Genes that Modulate Mitochondrial Stress

A high throughput screen for genes that modulate mitochondrial stress was performed. Cell lines are essential for high throughput screening; however, most lines show little mitochondrial activity as they are glycolytic and thus are unsuitable for such a screen. The E6/E7LOW hepatocyte cell line was previously produced (Levy et al., “Long-term culture and expansion of primary human hepatocytes”, Nat Biotechnol., 2015 December; 33(12):1264-1271) which continuously proliferates while maintaining an energetic state with significant mitochondrial activity. E6/E7LOW hepatocyte derived from a male and a female donor were both produced as it is known that gender plays a role in lipid metabolism and mitochondrial function.

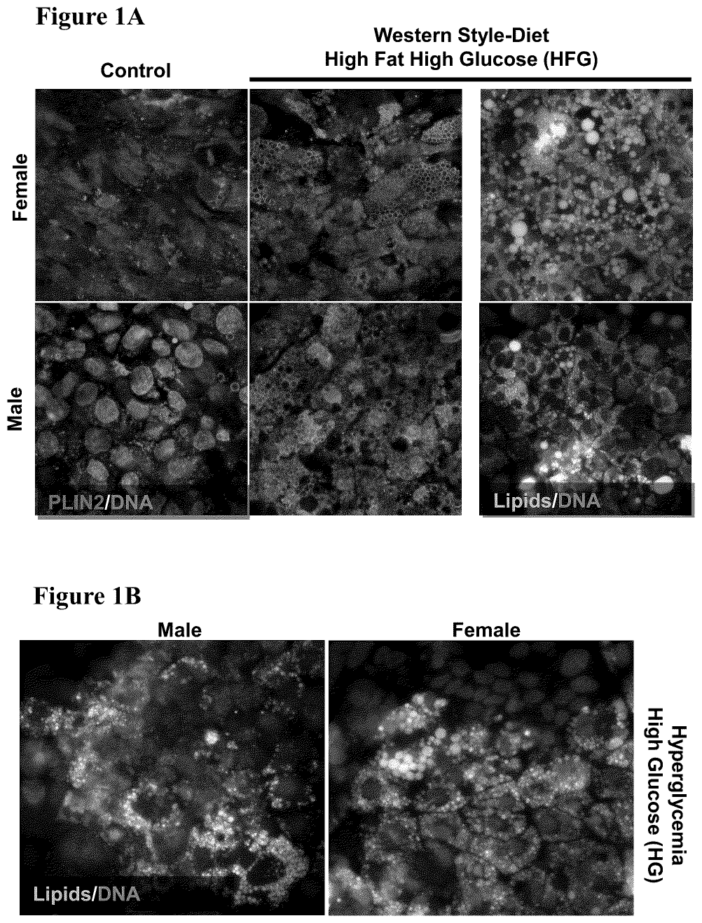

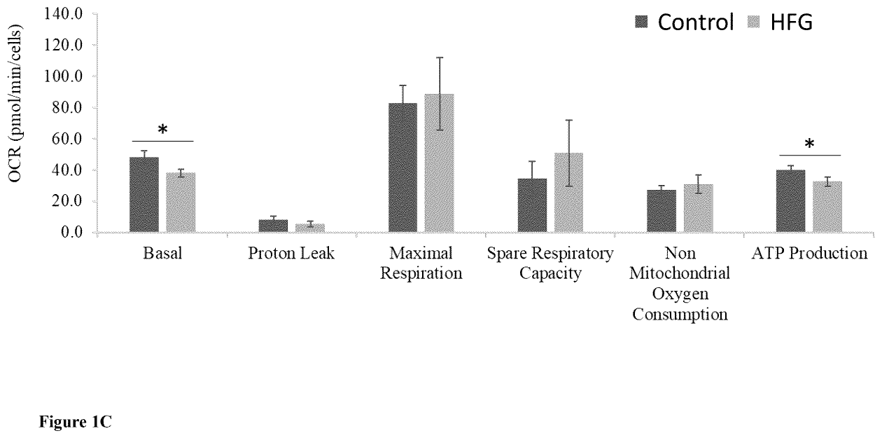

Steatosis was induced in E6/E7LOW cells by culturing them in a high fat and high glucose medium mimicking Western-style diet (HFG conditions) (FIG. 1A). A high glucose medium mimicking diabetes (HG conditions) also produced steatosis (FIG. 1B). A bioenergetic analysis of control and HFG steatotic cells was performed to confirm that the hepatocytes remain energetic even after steatosis differentiation. Basal respiration and ATP production were significantly lower in hepatocytes exposed to HFG (FIG. 1C). Similar reduction of basal metabolic rate is observed in obese patients.

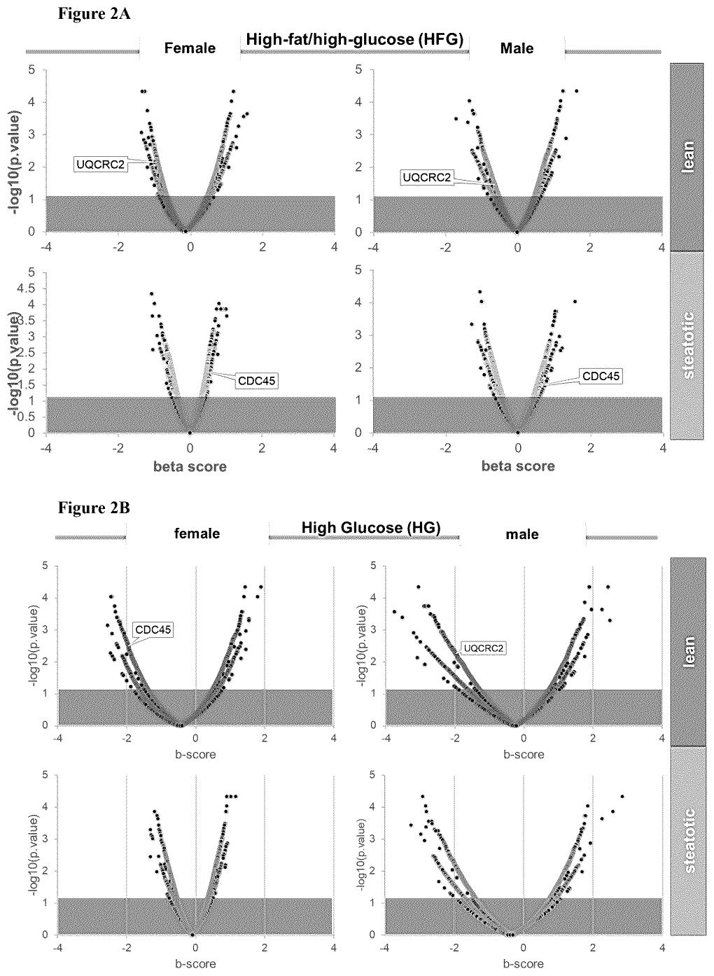

The steatotic cells were infected with a CRISPR lentiviral library (MOI ˜0.3) and selected with puromycin for expression of the sgRNAs. Following expansion, the cells were differentiated to steatosis with a 2-day culture in either HFG or HG medium. Control cells had no induction with special medium. The cells were then lipid-stained and sorted by FACS. The 14% highest intensity cells were sorted as the “steatotic bin” and the 25% lowest intensity cells were sorted as the “lean bin”. sgRNA abundance in the two bins was then evaluated. sgRNA enrichment/depletion was calculated based on respective NGS reads processed through the MAGeCK pipeline (M&M). Genes whose sgRNAs (knockdown) were enriched in the steatotic bin and genes whose sgRNAs were depleted in the lean bin are targets whose expression reduces lipid accumulation/steatosis. FIG. 2A shows volcano plots of enriched/depleted sgRNAs for the HFG differentiation. 2 high-ranking genes are noted, along with the 0.05 threshold of significance. FIG. 2B shows volcano plots of enriched/depleted sgRNAs for the HG differentiation. The same 2 high-ranking genes are noted, along with the 0.05 threshold of significance.

Pathway enrichment analysis was done for all enriched sgRNAs in both HFG and HG differentiation (FIG. 3A). Cell cycles, stress response, DNA/RNA metabolism predominate. Highest ranking genes were selected for further testing. These genes and their beta-score and p-values are presented for each condition tested, each sex and each bin in FIG. 3B.

### Example 2: Gene Validation

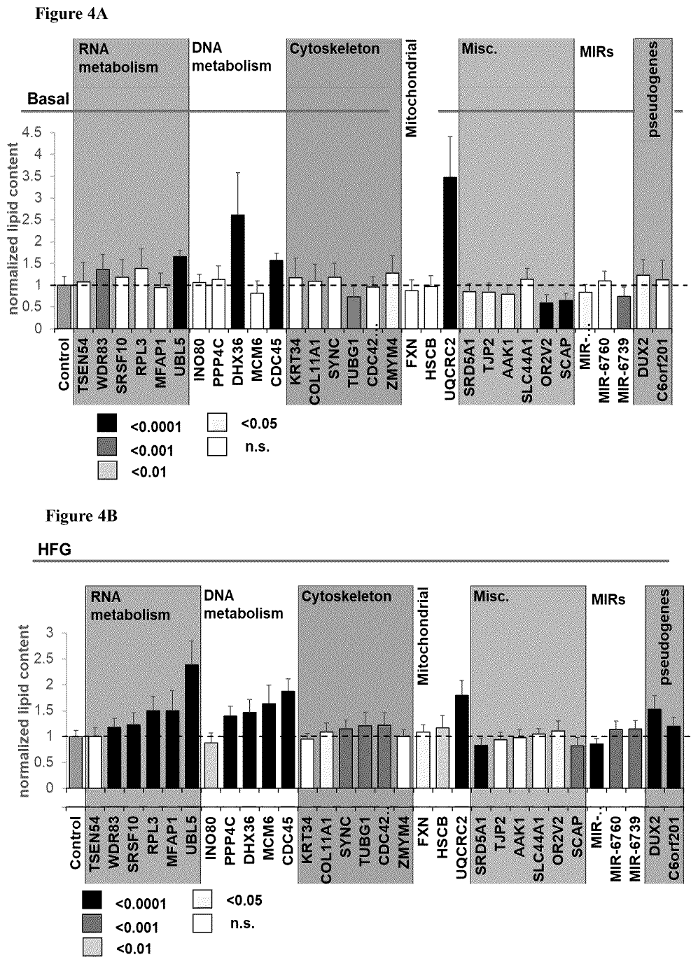

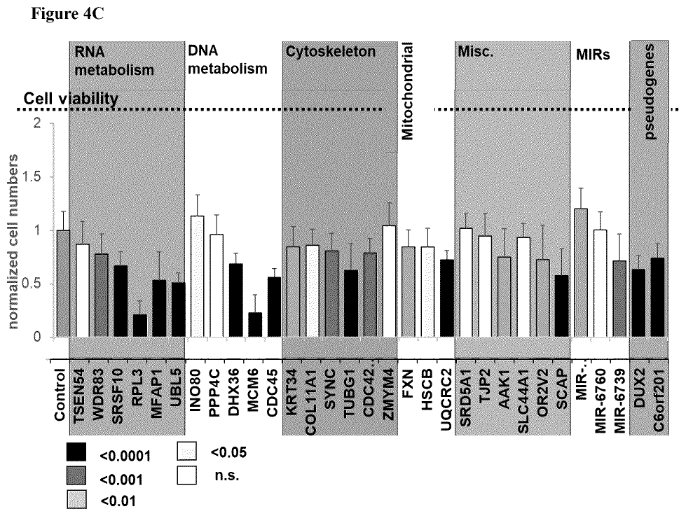

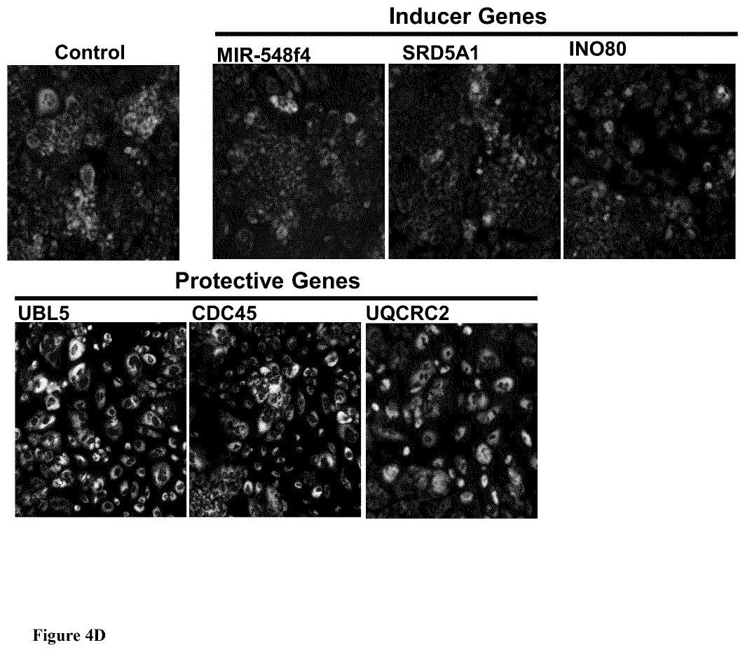

31 genes were now validated as targets for reducing lipid accumulation and improving metabolic/mitochondrial function. The genes were knocked out in female hepatocytes using individual lentiviruses (2×sgRNA per gene). After HFG culture, the cells were stained for lipids and fluorescent intensity was quantified. The normalized lipid content was calculated for each gene both in basal conditions (normal diet) (FIG. 4A) and after HFG (western-style diet) (FIG. 4B). Cell viability was also calculated for the knockouts and knockout of several genes indeed reduced viability (RPL3, MFAP1, UBL5, MCM6, and CDC45) (FIG. 4C). Knockout of UBL5, CDC45 and UQCRC were found to cause the highest lipid accumulation, while knockout of MIR-548f4, SRD5A1 and IN080 caused the greatest reduction in lipid accumulation after HFG (FIG. 4D).

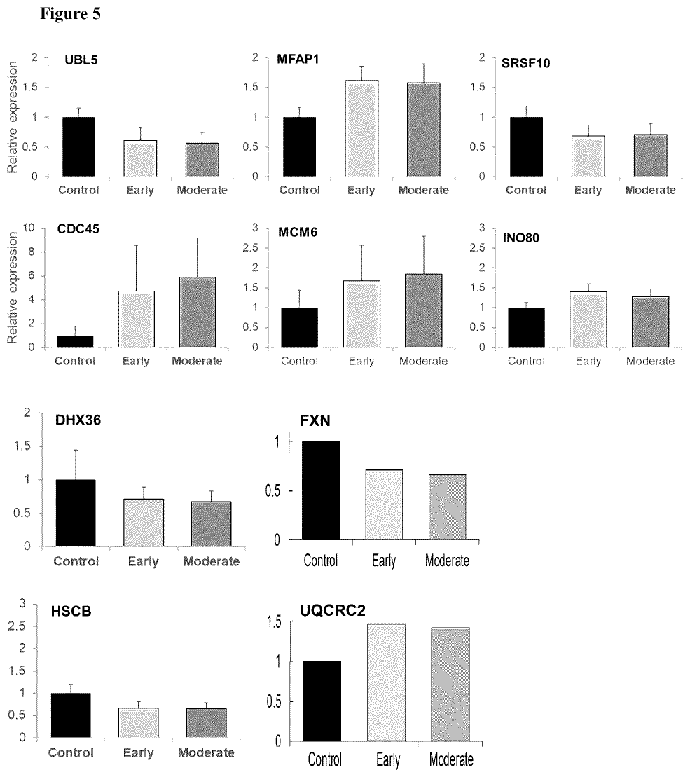

Non-alcoholic fatty liver disease (NAFLD) is characterized by an over nutrition-associated mitochondrial stress. Liver biopsies from early stage and moderate NAFLD patients (Early NAFLD, n=138; Moderate NAFLD, n=68) were compared to control liver biopsies (n-10) with respect to expression of several of genes found to have a significant in vitro effect on lipid accumulation (FIG. 5). Many of these genes were indeed differentially expressed in the NAFLD liver samples, with UBL5, SRSF10, DHX36, FXN and HSCB downregulated during NAFLD and CDC45, MCM6, IN080, UQCRC2 and MFAP1 upregulated. This validates these genes are genuine targets that regulate lipid accumulation and metabolic/mitochondrial function.

### Example 3: Overexpression Reduces Lipid Accumulation

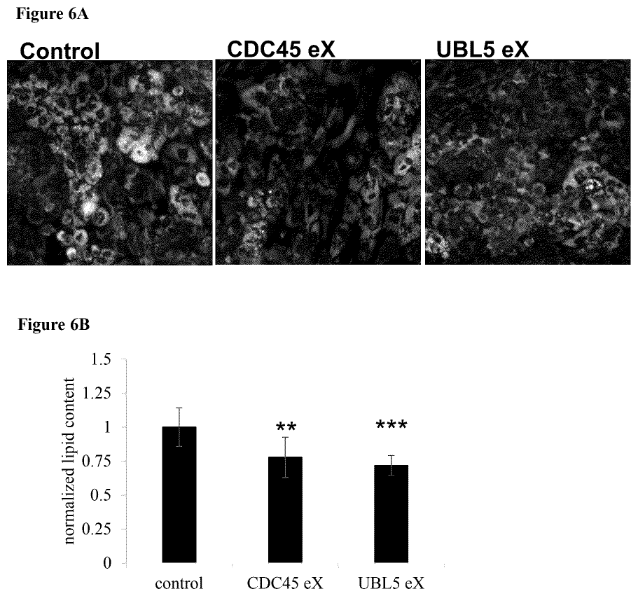

UBL5, CDC45 and UQCRC2 were selected as the most promising therapeutic targets. To test this, the three genes were each ectopically overexpressed the gene using a genomic insert that contained the native cDNA sequence and the e2IF promoter. The sequence was inserted using a lentivirus vector (nanoparticle) in hepatocytes which were then subjected to HFG culture. As predicted, overexpression of UBL5 and CDC45 reduced lipid accumulation during HFG culture (FIG. 6A) and this reduction was statistically significant (FIG. 6B). Similar results were observed for UQCRC2 overexpression (data not shown). This data supports the use of UBL5, CDC45 and UQCRC2 overexpression as a therapeutic treatment for disease characterized by increased lipid accumulation.

### Example 4: Overexpression of UBL5 to Treat Bone Marrow Failure

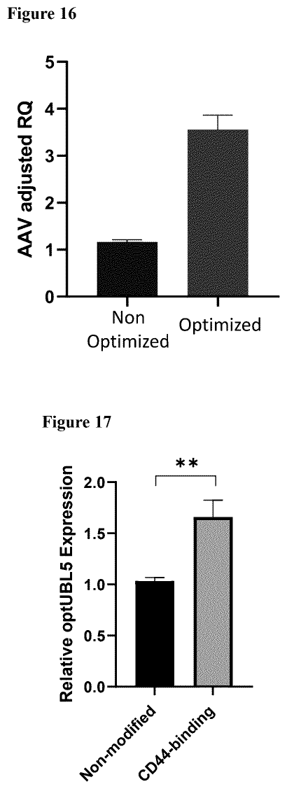

UBL5 was selected as the most promising therapeutic gene and thus was used going forward. UBL5 is a very short gene (only 219 bases) thus making it ideal for packaging into an adeno-associated viral delivery vector (AAV). AAVs are currently in use for gene therapy and allow for the delivery of genetic material to human cells in vivo. To this end an optimized UBL5 sequence was generated. Even though the UBL5 sequence is a human sequence, it was codon optimized for AAV expression. As the virus was produced in insect cells it was also optimized to remove splice sites and error prone sites. The optimized UBL5 coding sequence is provided in SEQ ID NO: 18. The optimized sequence has a greatly increased GC content (47.75% vs. 52.25%), a greatly increased CAI (0.76 vs. 0.96) and the yield of virus produced was also increased (FIG. 16). Further, the optimized UBL5 contains no cryptic splice sites and no common mutation and error-prone sites.

Bone marrow failure (BMF) diseases are characterized by degenerative deterioration in bone marrow state, development of anemia and emergence of multiple malignancies. It is well established that DNA damage is often the cause of BMF. Fanconi Anemia (FA) is the best studied BMF disease and thus was selected as the model for this class of conditions. Over 80% of FA cases are due to mutations in one of the Fanconi anemia complementation group genes: FANCA, FANCC and FANCG. Loss of function in a FANC protein results in defective DNA damage response and mitochondrial stress. This stress causes bone marrow lipotoxicity and failure. UBL5 is known to participate in Fanconi DNA repair complexes. It was hypothesized that ectopic expression of UBL5 can reestablish a healthy mitochondrial unfolded protein response (UPR-MT) and resolve BMF.

To test this hypothesis, the optimized UBL5 coding sequence was inserted into a rAAV9 vector. In order to increase this vector's specificity to primary immune cells, CD44 targeting peptides were designed in silico (SEQ ID NO: 19-29). Over 1490 possible peptides were evaluated and the 11 peptides with the strongest predicted binding to CD44 (<−21 change in free energy) are provided in Table 1. A sequence encoding the highest binding CD44 targeting peptide (YNGTIFF, SEQ ID NO: 19) was inserted in the AAV capsid coding region. Specifically, the septapeptide was inserted between glutamine 588 and alanine 589 in the coat protein domain of the capsid. The specificity of the virus to CD44 and primary immune cells was confirmed by infecting PBMCs with unmodified and modified AAV.

CD44 positive cells were then isolated and UBL5 expression was measured. Expression was significantly increased in the CD44 positive cells infected with virus comprising the CD44 binding peptide as compared to the unmodified virus (FIG. 17). This viral construct was termed TD01-VVL5 and its biodistribution in mice was examined. High levels of virus reached the bone marrow with the tibia of the mice and low levels were observed in most major organs including lung, spleen, kidney, brain and liver.

TABLE 1
CD44 binding peptides and binding energy

| Sequence | SEQ ID NO: | Binding energy |
|---|---|---|
| YNGTIFF | 19 | −29.53337066 |
| RSIFFLK | 20 | −26.65336736 |
| LVSYFGI | 21 | −25.67173731 |
| NPIIFFL | 22 | −24.98792648 |
| YNGIIVF | 23 | −23.33791164 |
| LVPYNHI | 24 | −22.30608975 |
| LVSYNGM | 25 | −22.30608975 |
| VSYHGII | 26 | −21.83245436 |
| YNGIMFF | 27 | −21.83245436 |
| YNGIILF | 28 | −21.59540926 |
| GIQFFTK | 29 | −21.03577922 |

### Example 5: Overexpression of UBL5 Improves Fanconi Anemia Mitochondrial Stress

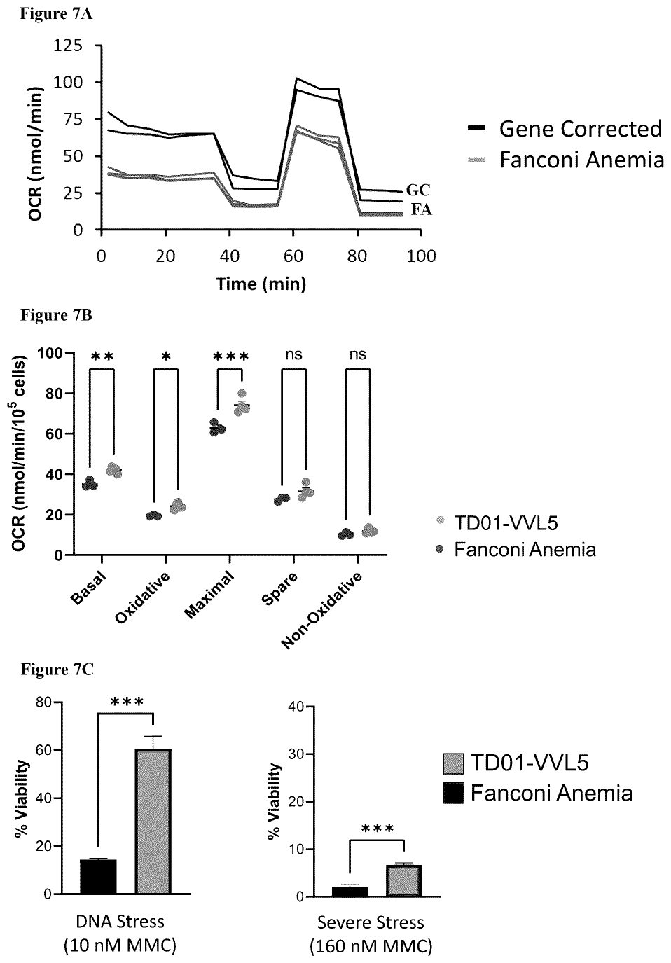

FA patient cells were obtained from the Fanconi Anemia Research Fund. The cells, which contained mutations in FANCA, FANCD and FANCG, showed phenotypic sensitivity to DNA damage (mitomycin; MMC) as assessed by cell viability with Presto blue (Thermo Fisher, Cat #-P50200). Analysis revealed that FA cells showed low basal metabolic rate and mitochondrial stress as compared to their gene-corrected counterparts (cells expressing a wildtype copy of the mutant gene) (FIG. 7A). FANCD2 mutant cells showed the most pronounced mitochondrial stress and so were administered TD01-VVL5. Ectopic expression of UBL5 reduced mitochondrial stress (FIG. 7B), significantly improved cell viability after DNA damage (4.3-fold increase, p<0.001 in both mild and severe DNA assays; FIG. 7C) significantly increasing basal respiration, oxidative respiration and maximal respiration capacity in the mutant cells (FIG. 7B).

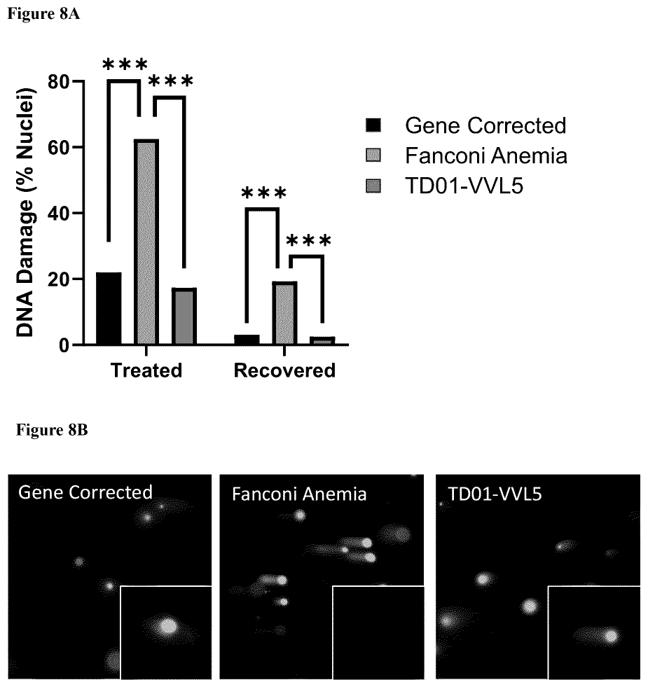

A COMET assay was performed to test DNA break repair and recovery in the FA cells, gene corrected cells and UBL5 treated cells. The cells were challenged with etoposide to inhibit DNA topoisomerase II activity causing DNA breaks and the assay was performed. DNA damage was seen in upwards of 60% of FA nuclei, while only 20% of gene corrected nuclei showed damage (FIG. 8A-B). This increase in damage was seen even after the cells were allowed to recover from the etoposide. Ectopic expression of UBL5 by TD01-VVL5 completely restored DNA break repair in the treated nuclei, decreasing DNA damage by greater than 3-fold and bringing damage levels down to those observed in the gene corrected nuclei (FIG. 8A-B).

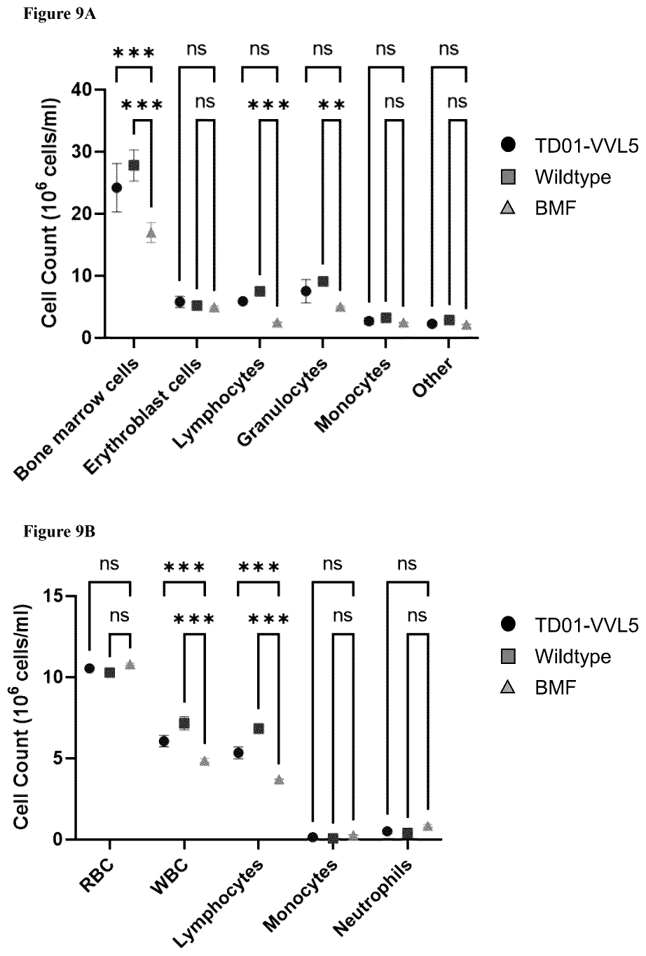

Leptin deficient mice were used as a model for obesity-induced stress. These mice have been shown to have bone marrow dysfunction, with disrupted blood cell homeostasis and impaired bone stem cell properties. It thus acts as a general model for BMF diseases and specifically for FA. Homozygous leptin knockout mice were injected with two doses of TD01-VVL5 over the course of 4 weeks (1 week acclimation followed by first injection, 1 week followed by second injection and then 2 weeks until the end of the experiment). After this, bone marrow was examined for overall density, composition and white cell count. Wildtype mice and mutant mice that did not receive the virus were used as controls. Bone marrow cell density was greatly reduced in the mutant mice, and a reduction in lymphocytes was also observed (FIG. 9A). Treatment with UBL5 significantly increased the number of bone marrow cells and the number of lymphocytes. Though this treatment did not return the counts exactly to the levels observed in the wildtype mice, there was not a significantly significant difference. A more in-depth counting of various blood cells found only white blood cells and specifically lymphocytes were reduced in the BMF mice and that both of these populations were increased in the treatment group (FIG. 9B).

In summary, TD01-VVL5 is administered systemically, e.g., intravenously, and ectopic expression of UBL5 in the bone marrow is identified. Mice administered the virus show improved bone marrow function and morphology. Reduced symptoms of BMF are observed. When all of this data is considered. it is clear that ectopic overexpression of UBL5 can successfully treat BMF diseases in general and FA in particular.

### Example 6: Overexpression of UBL5 Treats Overnutrition Induced NAFLD, Obesity and Diabetes

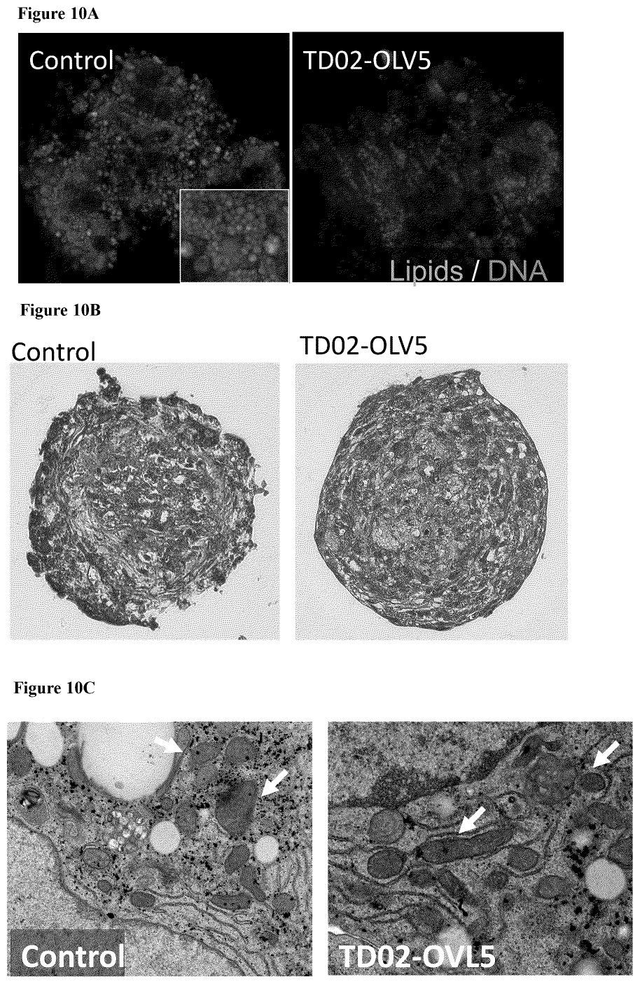

The ability of ectopic UBL5 overexpression to treat NAFLD, obesity and diabetes was also tested. To this end, the optimized UBL5 coding region was integrated into an rAAV9 virus without addition of a CD44-targeting peptide. This viral construct was called TD02-OVL5 and it produced broad ectopic expression of UBL5. Over nutrition induced fatty liver disease was tested in a human model. A library of E6/E7LOW hepatocytes representing European, Hispanic, and Asian genetic backgrounds were used to generate complex 3-dimensional human liver organoids with in vivo levels of drug and lipid metabolism (see, Levy et al., 2015, “Long-term culture and expansion of primary human hepatocytes”, Nature Biotechnology. 2015; 33(12):1264-1271, the contents of which are hereby incorporated herein by reference in their entirety). Western diet induced fatty liver disease shows micro-vesicular steatosis, proteolysis, mitochondrial stress, and inflammatory cytokine production in vitro. Lipid accumulation in the organoids was observed within 3 weeks (FIG. 10A) but was significantly reduced (greater than 4-fold, p<0.001) in TD02-OVL5 administered organoids (FIG. 10A). Human liver organoids exposed to dietary lipids showed collagen deposition indicative of fibrosis. Administration of TD02-OVL5 to the human liver organoids resolved fibrosis (FIG. 10B). Human liver organoids exposed to dietary lipids showed mitochondrial swelling as measured by transmission electron microscopy (TEM) and decreased respiration indicative of mitochondrial stress. Administration of TD02-OVL5 to the human liver organoids resolved mitochondrial swelling/stress (FIG. 10C).

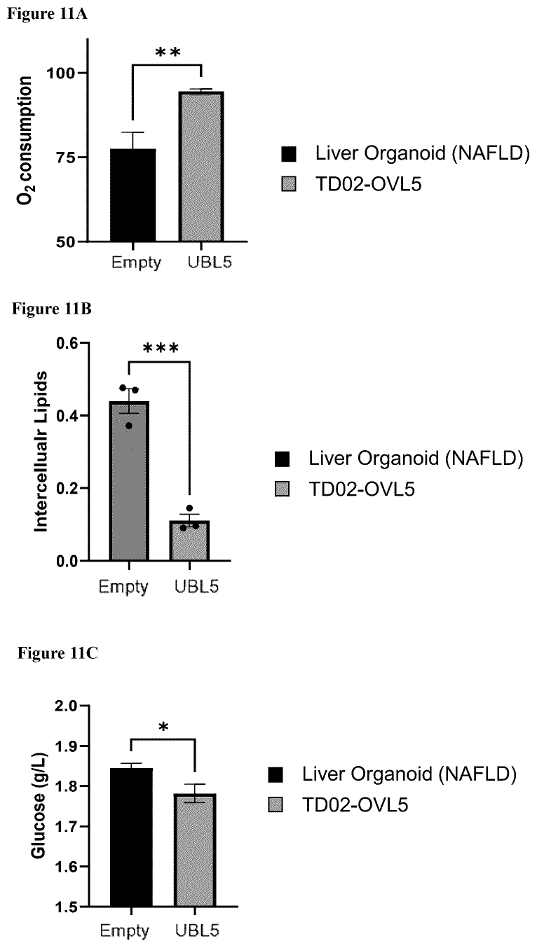

Basal metabolic rate of human liver organoids exposed to western-style diet decreased. The administration of TD02-OVL5 significantly increased oxygen consumption (FIG. 11A). Intracellular lipids (FIG. 11B) and glucose uptake rates (FIG. 11C) were similarly affected. Lipids accumulation decreased following UBL5 expression while glucose uptake rates increased.

### Example 7: Overexpression of UBL5 Treats Obesity and Diabetes

Western style diet not only induces inflammation and mitochondrial stress but also has led to a pandemic of obesity. GLP-1 agonists have recently been shown to reduce overeating, limit inflammation and thus indirectly improve aspects of mitochondrial function. However, these agents don't stimulate mitochondrial regeneration, thus limiting their potential efficacy. The human liver organoids produced show liver specific function for over 90 days in culture with complex metabolic zonation and native, human-specific ultrastructure and gene expression. In order to treat the systemic conditions of obesity and diabetes, a third viral construct was generated called TD02-OVL5.

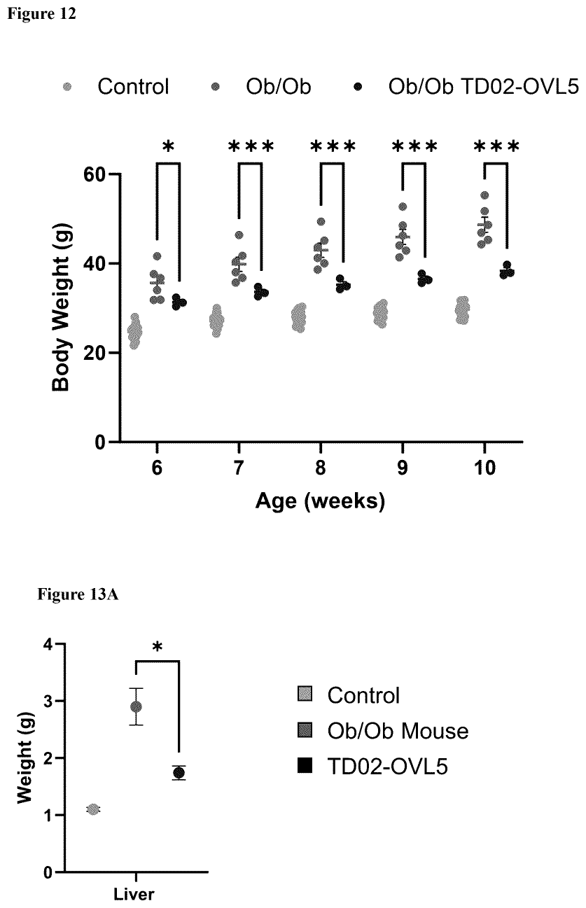

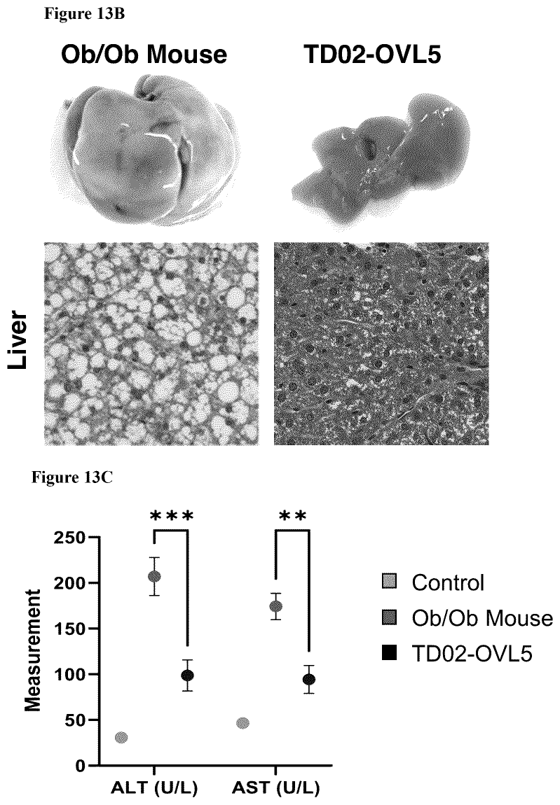

Leptin deficient mice (Ob/Ob mice) were used as a model for obesity. Starting with 6-week old mice, the mutant mice were acclimatized for one week and then treated with two tail vein injections of TD02-OVL5 a week apart. Two weeks after the second injection mice were weighed. As expected, the leptin deficient mice were significantly heavier than their wildtype counterparts. Mice treated with TD02-OVL5 showed a statistically significant reduction in weight gain (24%) compared to the untreated mutant mice (FIG. 12). The weight of the treated mice was not significantly different from control mice. TD02-OVL5 treatment also resolved steatohepatitis, restoring liver weight (FIG. 13A) and morphology (FIG. 13B) and reducing liver enzyme levels (FIG. 13C)

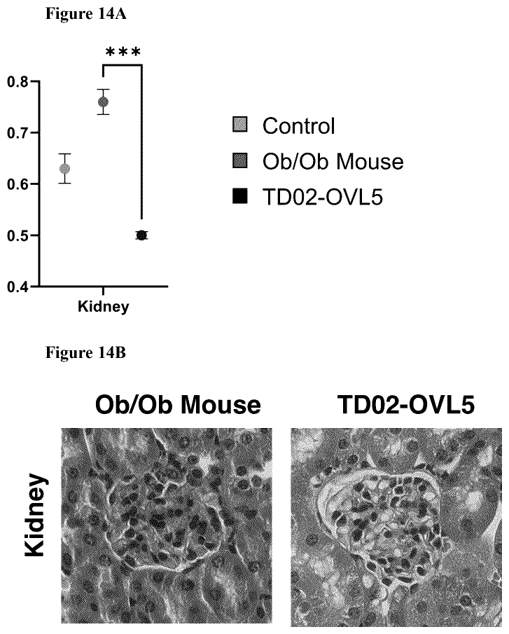

Kidney weight, which was also increased in the leptin deficient mice due to diabetes nephropathy, was reduced by TD02-OVL5 (FIG. 14A) and kidney morphology was returned to normal (FIG. 14B).

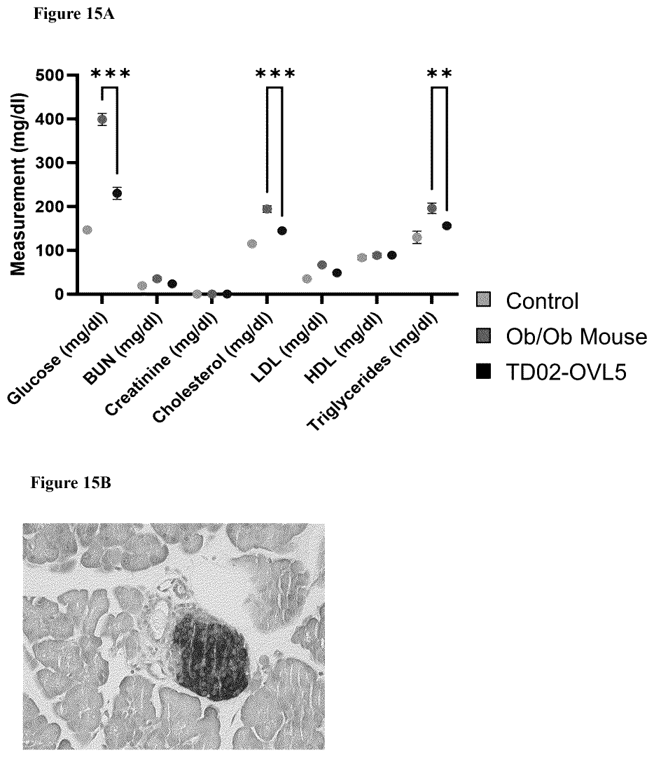

Leptin deficient mice also serve as a model for diabetes due to the systemic inflammation and resultant insulin resistance. TD02-OVL5 treatment reduced circulating glucose levels by 45% and circulating cholesterol levels by 25% as compared to mice that received a control empty virus (FIG. 15A). Further, histological examination of pancreatic islets showed restoration of normal morphology and organization (FIG. 15B). It is thus apparent that ectopic UBL5 overexpression can not only treat obesity but also insulin resistance.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A nanoparticle comprising a shell and an aqueous core, wherein said aqueous core comprises at least one of:
a. a nucleic acid molecule, wherein said nucleic acid molecule comprises a promoter operably linked to an open reading frame encoding human Ubiquitin-like protein 5 (UBL5); and
b. a human UBL5 polypeptide.

2. The nanoparticle of claim 1, wherein said human UBL5 comprises the amino acid sequence of SEQ ID NO: 9 or a variant thereof comprising at least 85% identity to SEQ ID NO: 9 and which retains unfolded protein response (UPR) functionality in mitochondria.

3. The nanoparticle of claim 1, wherein said open reading frame comprises the nucleotide sequence of SEQ ID NO: 1 or a variant thereof with at least 80% identity to SEQ ID NO: 1.

4. The nanoparticle of claim 1, wherein said open reading frame comprises the nucleotide sequence of SEQ ID NO: 18 or a variant thereof comprising at least 85% identity to SEQ ID NO: 18 and at least 80% identity to SEQ ID NO: 1.

5. The nanoparticle of claim 4, where said variant of SEQ ID NO: 18 encodes SEQ ID NO: 9.

6. The nanoparticle of claim 4, wherein said open reading frame consists of SEQ ID NO: 18.

7. The nanoparticle of claim 1, wherein said promoter is a heterologous promoter.

8. The nanoparticle of claim 1, wherein said nanoparticle is selected from a viral nanoparticle, a lipid nanoparticle and a synthetic nanoparticle.

9. The nanoparticle of claim 8, wherein said nanoparticle is an adeno associated viral (AAV) nanoparticle.

10. The nanoparticle of claim 9, wherein said AAV nanoparticle is an AAV9 nanoparticle.

11. The nanoparticle of claim 1, comprising a CD44 targeting peptide on said shell, wherein said CD44 targeting peptide is selected from YNGTIFF (SEQ ID NO: 19), RSIFFLK (SEQ ID NO: 20), LVSYFGI (SEQ ID NO: 21), NPIIFFL (SEQ ID NO: 22), YNGIIVF (SEQ ID NO: 23), LVPYNHI (SEQ ID NO: 24), LVSYNGM (SEQ ID NO: 25), VSYHGII (SEQ ID NO: 26), YNGIMFF (SEQ ID NO: 27), YNGIILF (SEQ ID NO: 28) and GIQFFTK (SEQ ID NO: 29).

12. The nanoparticle of claim 11, wherein said nanoparticle is a viral nanoparticle and said CD44 targeting peptide is inserted into a capsid of said viral nanoparticle.

13. The nanoparticle of claim 12, wherein said CD44 targeting peptide is inserted between glutamine 588 and alanine 589, and wherein positions are with respect to SEQ ID NO: 30.

14. The nanoparticle of claim 13, comprising a capsid fusion protein comprising said CD44 targeting peptide comprising an amino acid sequence selected from: SEQ ID NO: 31-41.

15. (canceled)

16. (canceled)

17. (canceled)

18. A pharmaceutical composition comprising a nanoparticle of claim 1 and a pharmaceutically acceptable carrier, excipient or adjuvant.

19. (canceled)

20. (canceled)

21. A method of treating a disease, disorder or condition characterized by mitochondrial stress in a subject in need thereof, the method comprising increasing expression of a protein selected from Ubiquitin-like protein 5 (UBL5), Cell division control protein 45 homolog (CDC45), Centrosomal protein of 295 kDa (CEP295), Superoxide dismutase [Mn], mitochondrial (SOD2), NADH dehydrogenase [ubiquinone]1 alpha subcomplex assembly factor 3 (NDUFAF3), Frataxin, mitochondrial (FXN), and Cytochrome b-cl complex subunit 2, mitochondrial (UQCRC2) in a diseased cell of said subject, thereby treating a disease characterized by mitochondrial stress, optionally wherein said subject is a human.

22. (canceled)

23. (canceled)

24. The method of claim 21, wherein said disease, disorder or condition is selected from a neuromuscular disease, an immune disease, a hematological disease, a cardiovascular disease, a neurodegenerative disease, a metabolic disorder or disease, a renal disorder, a dermatological condition, a cognitive disorder or a skeletomuscular condition.

25. (canceled)

26. (canceled)

27. The method of claim 24, wherein said disease, disorder or condition is a bone marrow failure disease or anemia or is selected from the group consisting of: Fanconi Anemia, Aplastic Anemia, Diamond-Blackfan Anemia, Dyskeratosis Congenita/Telomere Biology Disorders, GATA2 Deficiency, Myelodysplastic Syndrome, Paroxysmal Nocturnal Hemoglobinuria, Pearson's Disease, SAMD9/SAMD9L Germline Mutations, Severe Congenital Neutropenia, and Shwachman-Diamond Syndrome.

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. The method of claim 21, wherein said increasing comprises administering to said subject a pharmaceutical composition comprising a nanoparticle comprising a shell and an aqueous core, wherein said aqueous core comprises at least one of: a nucleic acid molecule, wherein said nucleic acid molecule comprises a promoter operatively linked to an open reading frame encoding said protein and said protein.

43. The method of claim 42, wherein said disease is a bone marrow failure disease and said nanoparticle comprises a CD44 targeting peptide on said shell, wherein said CD44 targeting peptide is selected from SEQ ID NO: 19-29.

44.-75. (canceled)
